(19) United States
(12) Patent Application Publication
(10) Pub. No.: US 2019/0142528 A1
(43) Pub. Date: May 16, 2019

(54) METHOD AND SYSTEM FOR IMAGE-GUIDED PROCEDURES

(71) Applicant: LX Medical Corporation, Westwood, MA (US)

(72) Inventor: Andrei VERTIKOV, Westwood, MA (US)

(21) Appl. No.: 16/300,475

(22) PCT Filed: May 23, 2017
(86) PCT No.: PCT/US2017/034002

§ 371 Date: Nov. 9, 2018

Related U.S. Application Data
(60) Provisional application No. 62/340,351, filed on May 23, 2016.

(51) Int. Cl. A61B 34/20; A61B 8/08; A61B 8/12

(52) U.S. Cl. CPC A61B 34/20; A61B 8/0841; A61B 8/12; A61B 8/488; A61B 2090/3784; A61B 2034/2051; A61B 2034/2063; A61B 2034/2065; A61B 2034/2048

(57) ABSTRACT

Image-guided systems and methods for enhanced medical tool tracking and guidance, virtual three-dimensional imaging, and enhanced fields of view. The system includes an imaging probe configured for side-view imaging. The guide sheath includes elongated flexible body and a channel configured to accept the imaging probe and a medical tool. The image console is configured to process imaging energy acquired by the image probe during side-view imaging to generate image data and calculate a global position of a distal end of the imaging probe or a distal end of the medical tool relative to a target in a tissue during imaging of the tissue using any one of 1D, 2D, and 3D sub-sets of the image data despite the target being located outside of fields of view of the imaging probe and not directly visualized by the imaging probe.

### CROSS-REFERENCE TO RELATED APPLICATIONS

The present application claims priority under 35 U.S.C. § 119 to U.S. Provisional Patent Application No. 62/340,351 filed on May 23, 2016, the entire contents of which is incorporated herein by reference.

### TECHNICAL FIELD

The present invention relates to the field of diagnostic medical imaging and image guidance for medical procedures. More specifically, the present invention relates to minimally invasive image-guided procedures in luminal anatomic structures, naturally or surgically created body cavities, or interstitially in tissue.

### BACKGROUND

Minimally-invasive procedures. The need for effective imaging for medical diagnostics as well as image guidance and control of diagnostic, therapeutic, and surgical procedures is well recognized. Often such imaging must be performed in a complex network of narrow and difficult-to-reach body lumens (such as, for example, blood vessels of cardiovascular and neurovascular systems, an airway tree of lungs, and gastrointestinal, bile, and urinary tracts) or in tight spaces of natural or surgically created body cavities. In general terms, image guidance is aimed at identifying and localizing a specific target within a patient body to allow for accurately placing a medical instrument or tool in this target to perform a medical procedure with the tool while avoiding anatomical risk structures. Many medical tools exist with specific intended uses for specific medical procedures such as, for example: an injection needle that delivers therapeutic agents; a biopsy instrument that takes tissue samples, such as an aspiration needle, biopsy forceps, or brush; a surgical instrument for diseased tissue resection; and a probe that deposits radiofrequency (RF), microwave (MW), laser, and/or other energy or cryogenically cools the tissue for diseased tissue ablation.

Existing flexible and rigid endoscopic imaging devices, such as bronchoscopes, thoracoscopes, laparoscopes, and alike, may help address the need for minimally-invasive image guided procedures utilizing various medical tools. Some endoscopes incorporate small cameras and illumination fibers at their distal ends together with working channels for deployment of medical tools under visual guidance. Other endoscopes are configured to work with tools that are deployed via separate incisions or surgically created openings.

Insertion widths vs. functionality. The size of lumens, natural cavities, and surgically created openings limits insertion widths of endoscopes and endoscopically deployed tools. In turn, these constraints limit functionality of the tools, their efficacy, and safety of their use. Thus, there is a trade-off between tool miniaturization and performance. For example, biopsy accuracy depends on the amount of sample tissue collected and this amount is proportional to a cross-sectional area of the internal lumen of a sample collecting instrument. Thus, there is a need for optimal configurations and optimal arrangements of tools in the tight spaces of minimally invasive procedures, together with a need for optimal methods of image guidance of these tools.

Robotically-assisted medical instruments. Medical procedures need to be performed on increasingly small anatomical structures or within small targets where robotically-assisted tool manipulation can be more accurate than manual handling. Such motorized tool actuation can also minimize surgeon exposure to radiation during intraoperative imaging and minimize the effects of surgeon fatigue. However, accurate actuation of flexible'tools deployed in torturous paths, as well as manipulation of tools in soft tissue, is challenging. In these cases, tool position feedback distally, at the deployment side, would improve procedure efficacy.

Multi-modality imaging. Often, several imaging modalities need to be combined for effective image guidance. For example, a target tissue can be located deep under an observed surface and, thus, cannot be easily visualized with standard endoscopy. In this case, subsurface imaging modalities such as, for example, endoscopic ultrasound might help to localize and guide tools to the target. Accordingly, some existing endoscopes include integrated ultrasound imaging or an ability to accept endoscopic ultrasound probes. For example, flexible endobronchial ultrasound probes (EBUS) for use in bronchoscopic image guided procedures exist. Also, flexible endoscopes exist with integrated curvilinear EBUS imaging to visualize and guide tools for transbronchial needle aspiration (TBNA). More recently, flexible Optical Coherence Tomography (OCT) imaging probes have been developed for sub-surface imaging, which can provide higher resolution images compared to ultrasound. However, integrating additional modalities into an endoscope increases the dimensions of its distal end, thus limiting its use in smaller lumens or cavities. Further, using separate imaging probes in endoscopically guided procedures may require an exchange of tools that share the same working channel. This tool exchange increases medical procedure duration, thus increasing patient distress, and also affects the accuracy of tool placement relative to a target.

Pre-operative vs. intra-operative imaging. Many medical procedures start with a planning phase that uses preoperative, or pre-procedural, image data, for example obtained with computed tomography (CT) or magnetic resonance imaging (MRI), to construct a tool trajectory for deployment to a target. Often, higher resolution intra-operative or intra-procedural data needs to be superimposed, or registered, onto the pre-procedural data. The need for registration of pre-procedural and intra-procedural images also exists when efficacy of surgery or treatment needs to be evaluated with follow-up imaging. Significant challenges exist for such registration in soft tissue when such tissue can deform and organs can shift.

3D vs. 2D imaging. Full information about a three-dimensional (3D) scene is lost with two-dimensional (2D) imaging provided by standard endoscopes. This loss of information leads to possible guidance errors or a need to use suboptimal tool trajectories as 2D images are difficult to visualize and interpret by a physician when mental registration of the image on the screen to the volume inside the patient body is needed. For example, clinicians might have limited depth perception with endoscopes when placing a tool to a target. In another example, a 2D cross-sectional view provided by a radial EBUS probe to visualize a tissue target may not contain the full trajectory of a biopsy needle when deployed in a forward position along a longitudinal axis of the probe, thus exposing anatomical structures, such as blood vessels, to puncture risks. Thus, 3D imaging is advantageous over 2D imaging for medical procedure control and guidance. However, 3D imaging is inherently more complex and costly, in particular when 3D data needs to be acquired, processed, and rendered in real time. As such, there is typically a trade-off between a resolution of volumetric images and a speed at which such images can be updated.

Spatial relationships, morphometry, velocity, and flow measurement. Imaging modalities that determine a spatial relationship of image data with respect to an imaging device have several advantages in image guidance. In these modalities, exemplified by CT, MRI, ultrasound, or OCT, each pixel or voxel of image data has a determined and quantifiable position with respect to the imaging system. The knowledge of distance to each voxel of data enables image morphometry and quantification of tool position relative to the target. When these images are analyzed sequentially, measurements of tissue motion (or tool motion) or flow within tissue are possible, permitting detection of organ shifts and further differentiating risk structures. These motion measurements have been realized by analyzing image features changes, image voxels within featureless scenes with Doppler processing, or speckle patterns of an imaged scene. Accurate morphometry and motion measurements are difficult with existing methods for complex trajectories within moving soft tissue.

The present invention is intended to address these and other deficiencies of minimally invasive image guided procedures.

### SUMMARY

Embodiments of the invention generally provide an image-guided system that includes a medical tool, a tool handling arrangement with an imaging probe, and a system console with a data-processing unit. The image-guided system determines, in real time, a position of the medical tool relative to a target within a patient body. The position is then used to guide and control accurate placement of the medical tool to the target.

According to some embodiments, an image-guided system including an imaging probe, a guide sheath, and an imaging console is provided. The imaging probe is configured to image a tissue and includes an elongated flexible body having a proximal end, an opposite distal end, and an outer wall extending from the proximal end to the distal end, where at least a portion of the outer wall is at least partially transparent to imaging energy used for side-view imaging by the imaging probe. The imaging probe also includes an energy guide extended inside the flexible body and configured to deliver the imaging energy between the proximal end and the distal end, and at least one energy directing element configured to transmit the imaging energy delivered by the energy guide to the tissue. The guide sheath includes elongated flexible body with a proximal end, an opposite distal end, and a channel extending from the proximal end to the distal end, where the channel is configured to accept the imaging probe and a medical tool. The imaging console includes a data-processing unit in operable communication with the imaging probe. The image console is configured to process the imaging energy acquired by the image probe during side-view imaging to generate image data and calculate a global position of one of the distal end of the imaging probe and a distal end of the medical tool relative to a target in the tissue during imaging of the tissue using any one of 1D, 2D, and 3D sub-sets of the image data despite the target being located outside of fields of view of the imaging probe and not directly visualized by the imaging probe.

In some embodiments, an image-guided system including an imaging probe and an imaging console is provided. The imaging probe is configured to image a tissue and includes an elongated flexible body having a proximal end, an opposite distal end, and an outer wall extending from the proximal end to the distal end, where at least a portion of the outer wall is at least partially transparent to imaging energy used for side-view imaging by the imaging probe. The imaging probe also includes an energy guide extended inside the flexible body and configured to deliver the imaging energy between the proximal end and the distal end and at least one energy directing element configured to transmit the imaging energy delivered by the energy guide to the tissue. The imaging console is in operable communication with the imaging probe and is configured to process imaging energy acquired by the imaging probe to generate and store first image data, calculate a real-time position of the distal end of the imaging probe during imaging of the tissue using, any of 1D, 2D, and 3D sub-sets of the first image data, and render a virtual image of tissue around the distal end of the imaging probe by remapping pre-acquired reference image data of the tissue according to the real-time position of the distal end of the imaging probe.

In some embodiments, a method of guiding a medical tool to a target in a tissue using a system having a guide sheath, an imaging probe, and an imaging console communicating with the imaging probe is provided. The method includes positioning a distal end of the guide sheath into proximity to the target, positioning the medical tool in the guide sheath and advancing a distal end of the medical tool towards the target, and positioning a distal end Hof the imaging probe in the guide sheath and acquiring image data of tissue in proximity to the target and/or the medical tool. The method also includes determining position and orientation of the medical tool relative to the target by processing the image data acquired by the imaging probe using any of the following: processing Doppler shifts in 1D, 2D, and 3D sub-sets of the image data; processing speckles in the 1D, 2D, and 3D sub-sets of the image data; comparing the 1D, 2D, and 3D sub-sets of the image data with reference image data pre-acquired and stored in data processing memory of the imaging console; comparing tissue identifiers pre-computed from the reference image data pre-acquired and stored in data processing memory of the imaging console with tissue parameters determined from the image data; processing data acquired from an acceleration sensor disposed within the distal end of the imaging probe; processing data acquired from a magnetic sensor disposed within the distal end of the imaging probe, the magnetic sensor configured to sense a magnetic field generated by at least one coil positioned outside the tissue; and determining a bended shape of at least one of the guide sheath, the medical tool, and the imaging probe. The method further includes repositioning at least one of the guide sheath and the medical tool towards the target using the determined position and orientation of the medical tool relative to the target.

In some embodiments, a method of guiding a medical tool to a target in a tissue using a system having an imaging probe and an imaging console is provided. The method includes advancing a distal end of the medical tool towards the target, positioning a distal end of the imaging probe in the distal end of the medical tool, and acquiring image data of at least a portion of tissue in proximity to the target and/or the medical tool. The method also includes determining position and orientation of the medical tool relative to the target by processing the image data acquired by the imaging probe using any of the following: processing. Doppler shifts in 1D, 2D, and 3D sub-sets of the image data; processing speckles in the 1D, 2D, and 3D sub-sets of the image data; comparing the 1D, 2D, and 3D sub-sets of the image data with reference image data pre-acquired and stored in data processing memory of the imaging console; comparing tissue identifiers pre-computed from the reference image data pre-acquired and stored in data processing memory of the imaging console with tissue parameters determined from the image data; processing data acquired from an acceleration sensor disposed within the distal end of the imaging probe; processing data acquired from a magnetic sensor disposed within the distal end of the imaging probe, the magnetic sensor configured to sense a magnetic field generated by at least one coil positioned outside the tissue; and determining a bended shape of at least one of the guide sheath, the medical tool, and the imaging probe. The method further includes repositioning the medical tool towards the target using the determined position and orientation of the medical tool relative to the target.

In some embodiments, a method of extending field of view of imaging a tissue using a system having an imaging probe and an imaging console communicating with the imaging probe is provided. The method includes determining a real-time position and orientation of a distal end of the imaging probe relative to surrounding, tissue by processing image data acquired by the imaging probe, the processing including any one of: processing Doppler shifts in 1D, 2D, and 3D sub-sets of the image data; processing speckles in the 1D, 2D, and 3D sub-sets of the image data; comparing the 1D, 2D, and 3D sub-sets of the image data with reference image data pre-acquired and stored in data processing memory of the imaging console; comparing tissue identifiers pre-computed from the reference image data pre-acquired and stored in data processing memory of the imaging console with tissue parameters determined from the image data; processing data acquired from an acceleration sensor disposed within the distal end of the imaging probe; processing data acquired from a magnetic sensor disposed within the distal end of the imaging probe, the magnetic sensor configured to sense a magnetic field generated by at least one coil positioned outside the tissue; and determining a bended shape of at least one of a guide sheath, a medical tool, and the imaging probe. The method also includes rendering a virtual image of tissue around the distal end of the imaging probe by remapping the pre-acquired reference image data according to the real-time position of the distal end.

### BRIEF DESCRIPTION

The invention will be more fully understood by referring to the following Detailed Description in conjunction with the Drawings, of which:

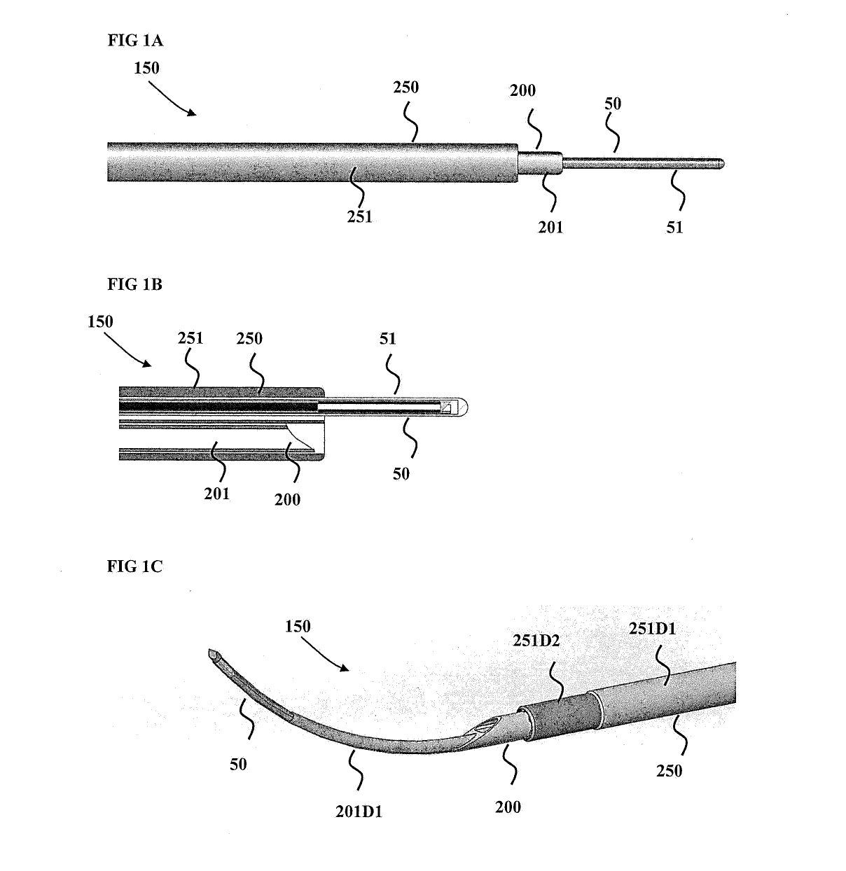

FIG. 1A illustrates a side view of a medical tool and an imaging probe substantially co-axially nested in a guide sheath. FIG. 1B illustrates a cross-sectional view of the imaging probe and the medical tool positioned in laterally offset manner. FIG. 1C illustrates a perspective view of a device distal end when a medical tool or an imaging probe is configured to be deployed non-collinear with respect to a guide sheath;

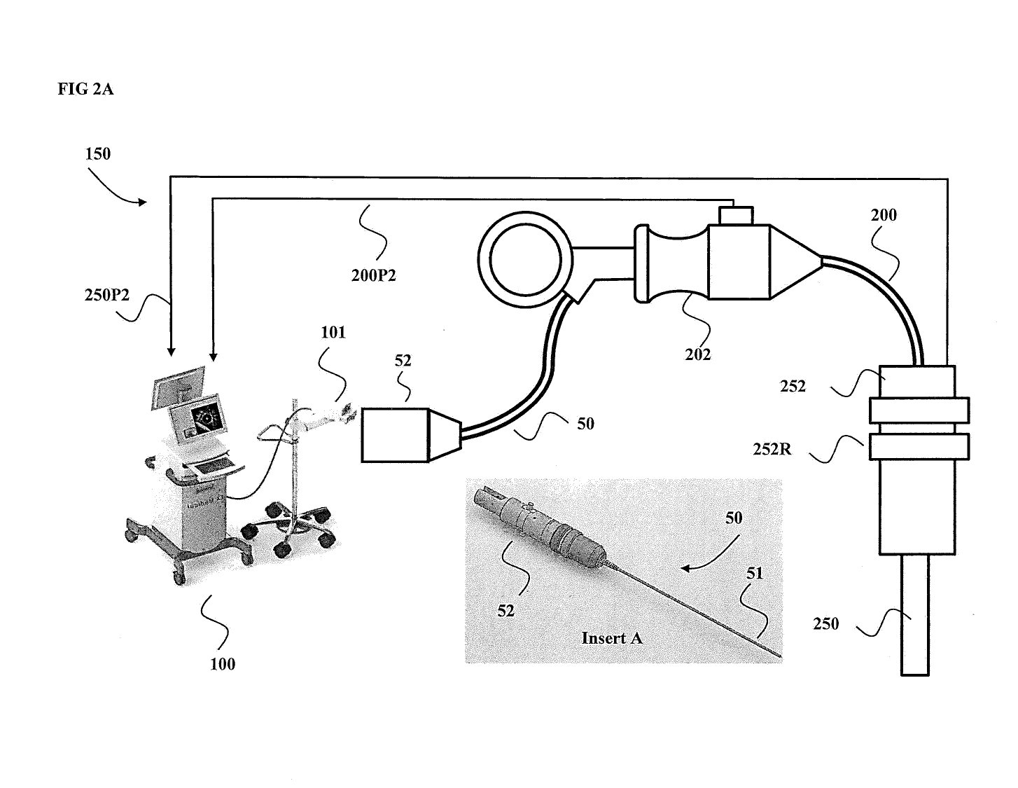

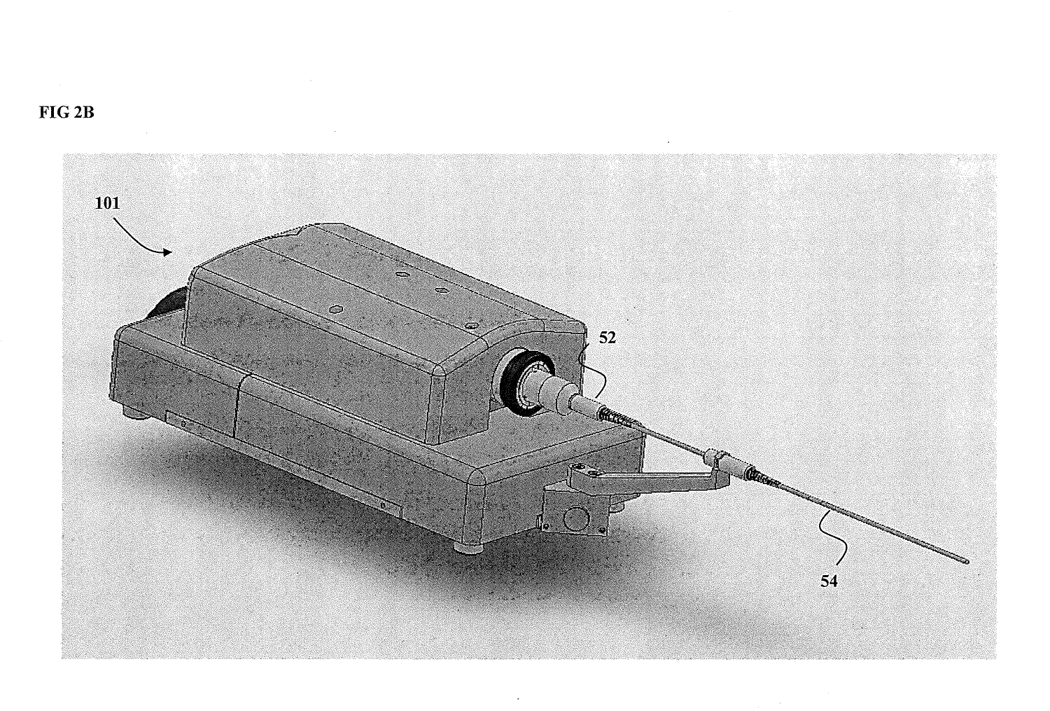

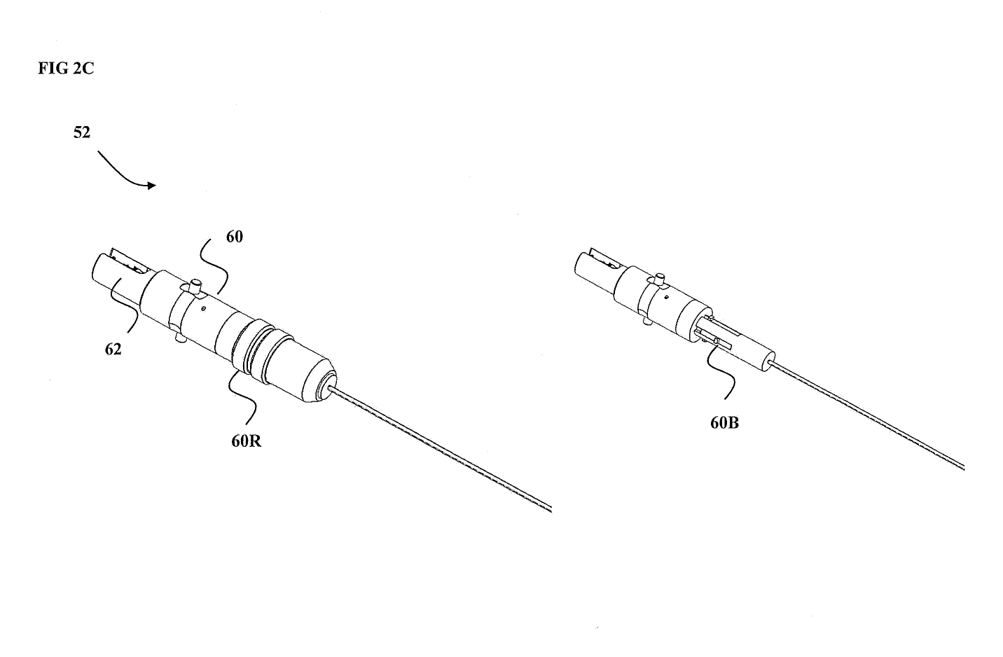

FIG. 2A illustrates a schematic view of a proximal end of a device interfaced with a system console of an image-guided system. FIG. 2B illustrates a perspective view of an imaging probe interfaced with a drive unit of the system console of FIG. 2A. FIG. 2C illustrates steering controls at the proximal end of a device;

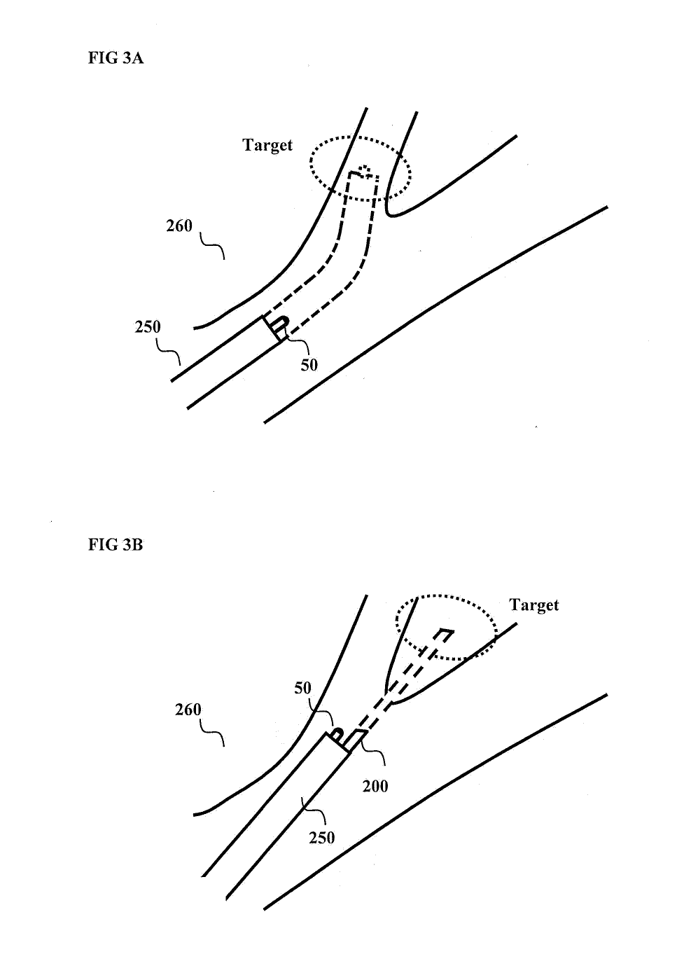

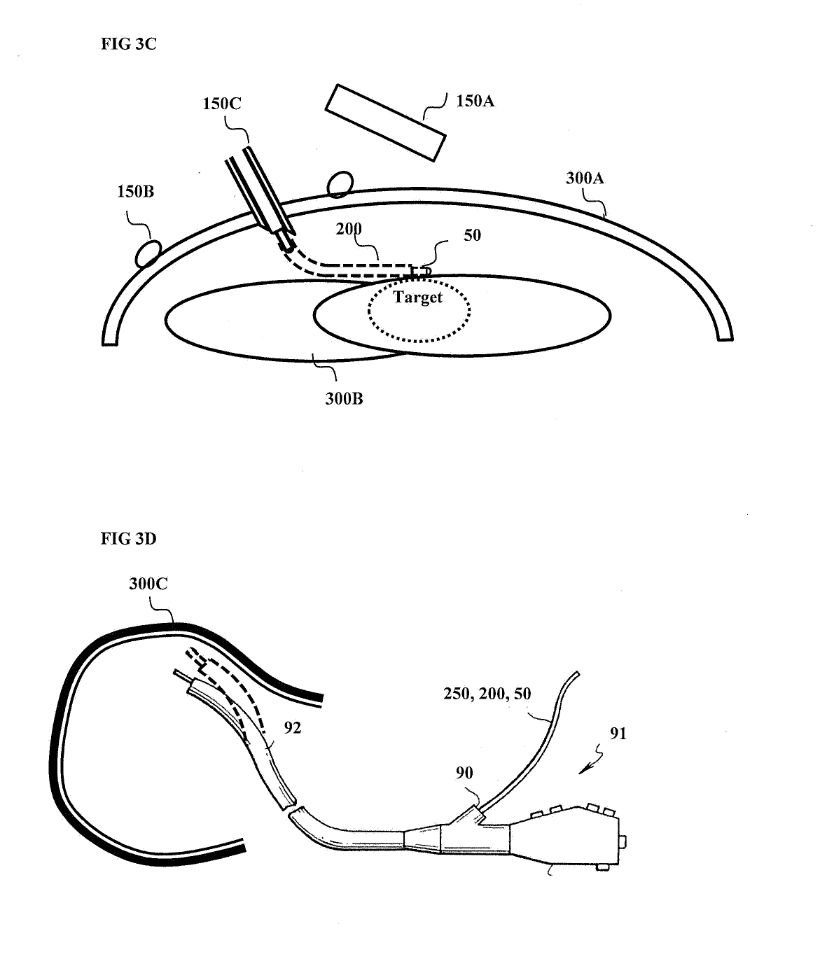

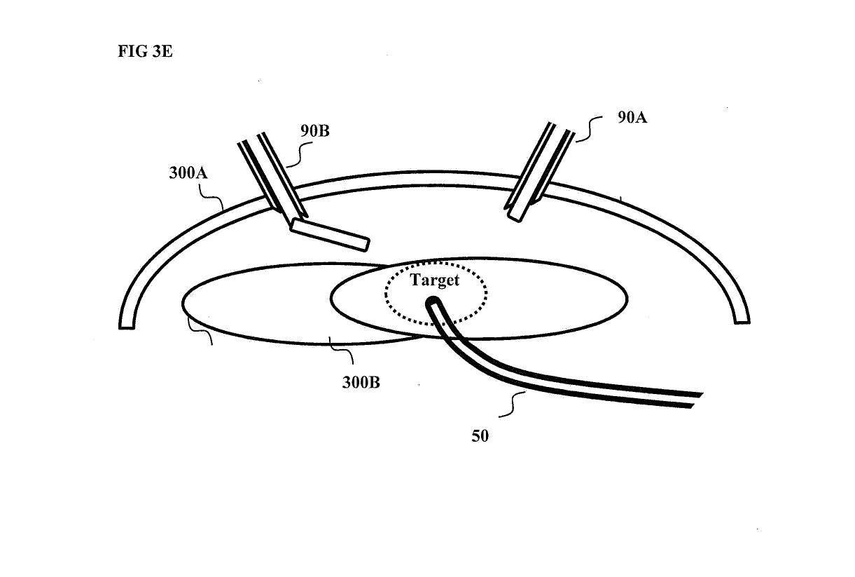

FIGS. 3A, 3B, 3C, 3D, and 3E are diagrams illustrating methods of delivery of medical tools to a target;

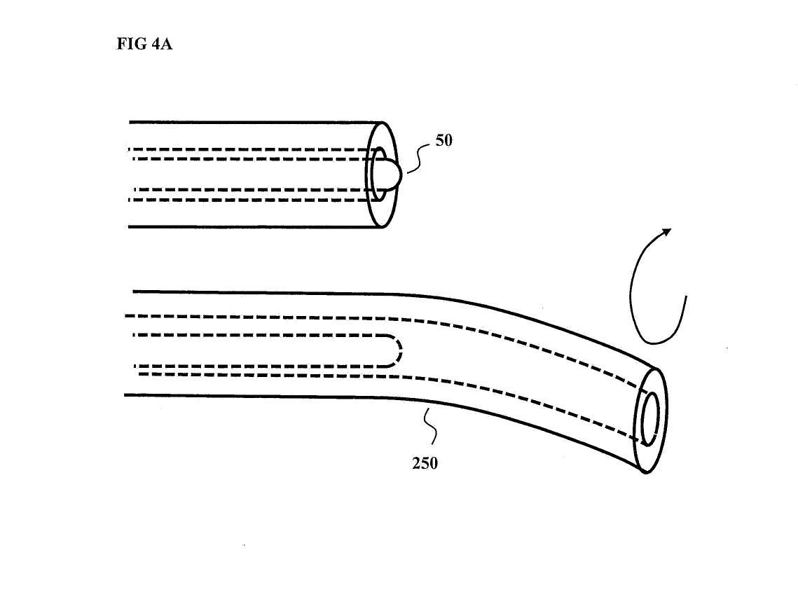

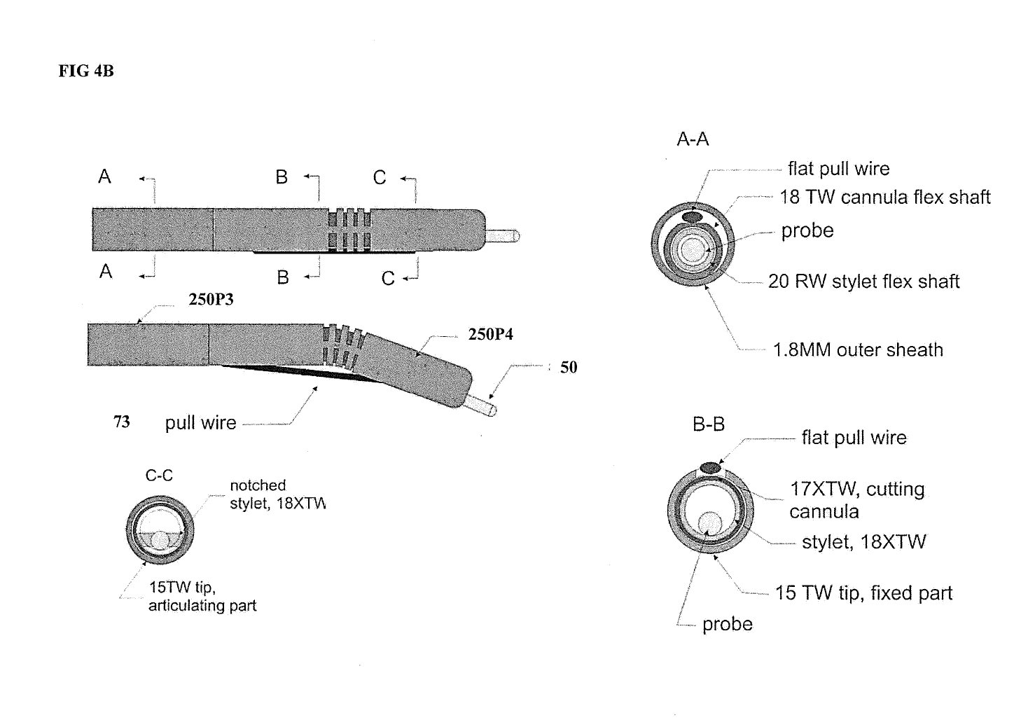

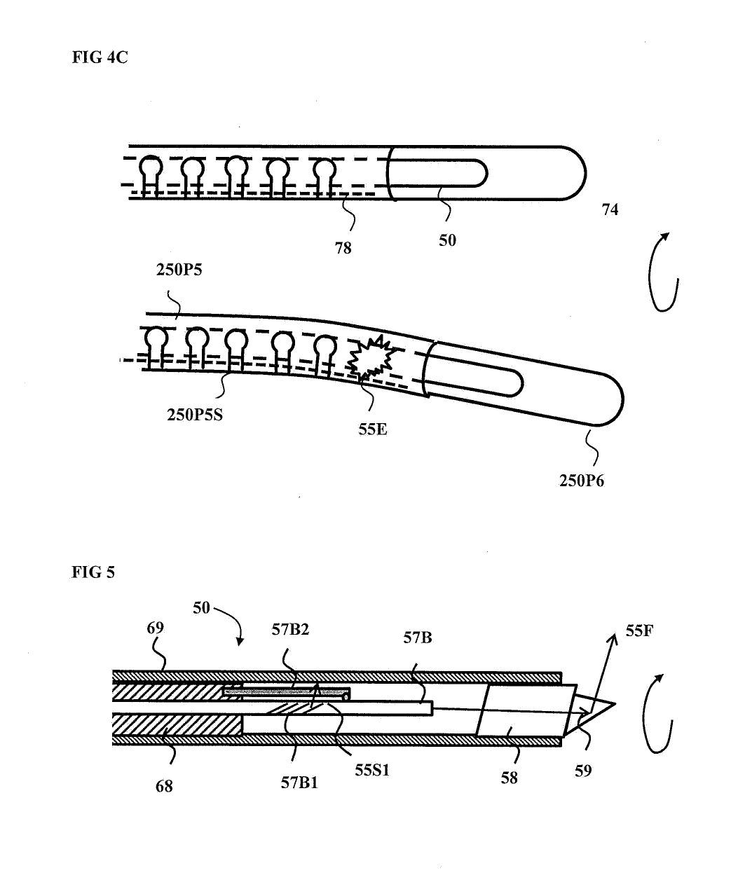

FIGS. 4A, 4B, and 4C show steering mechanisms used to deliver medical tools according to the methods of FIGS. 3A-3E;

FIG. 5 shows a cross-sectional view of a distal end of device including scanning arrangements of an imaging probe with an optical fiber dithered or controlled by a light activated actuator;

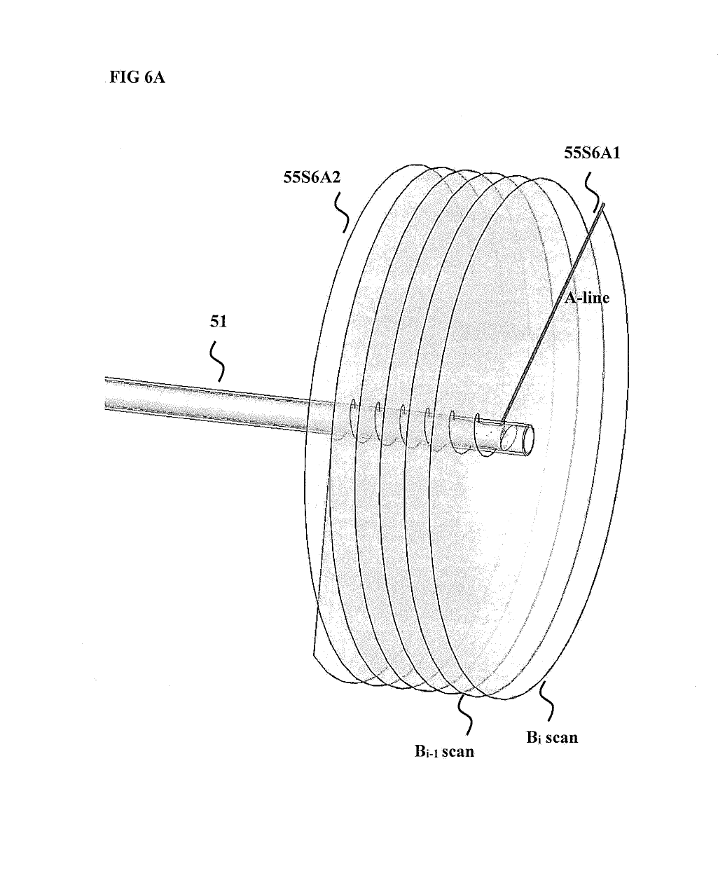

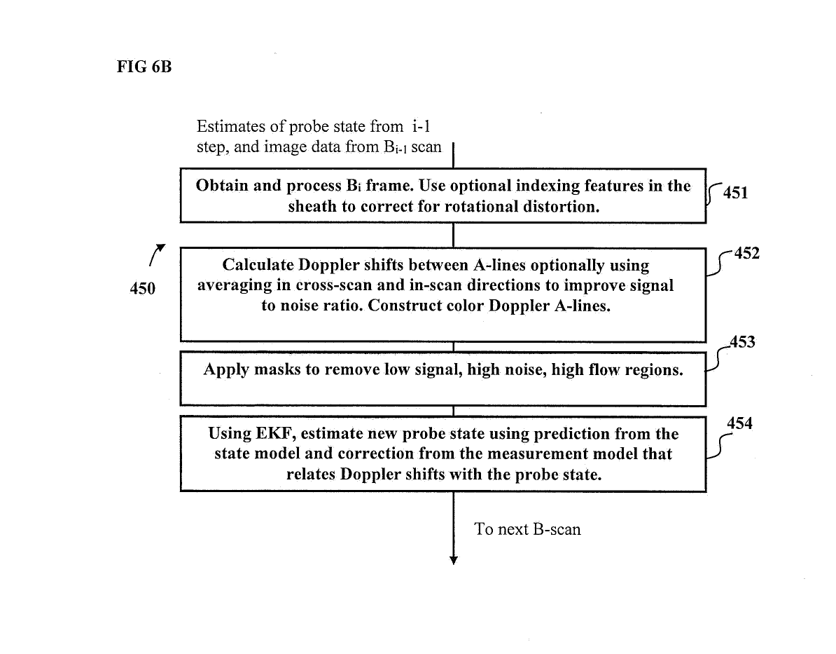

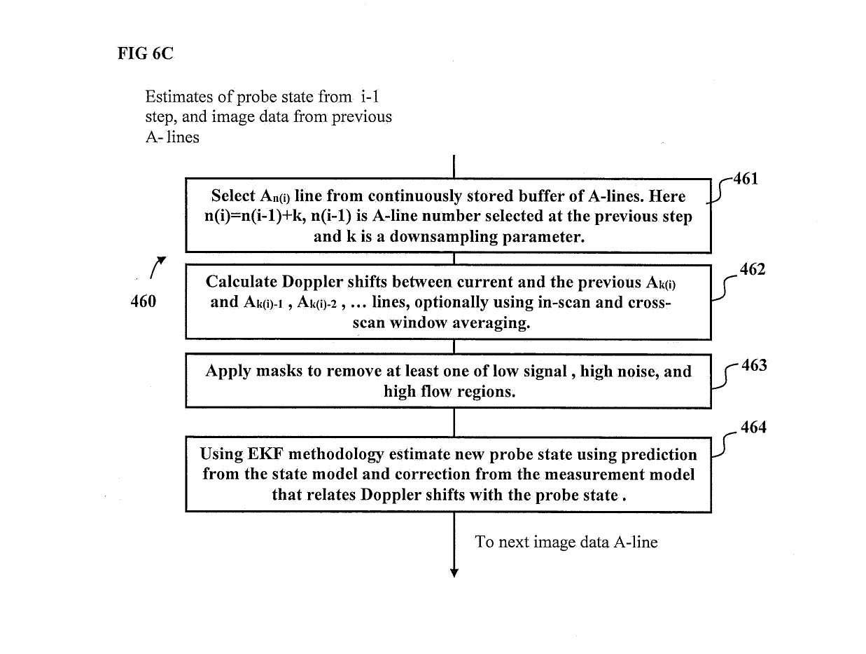

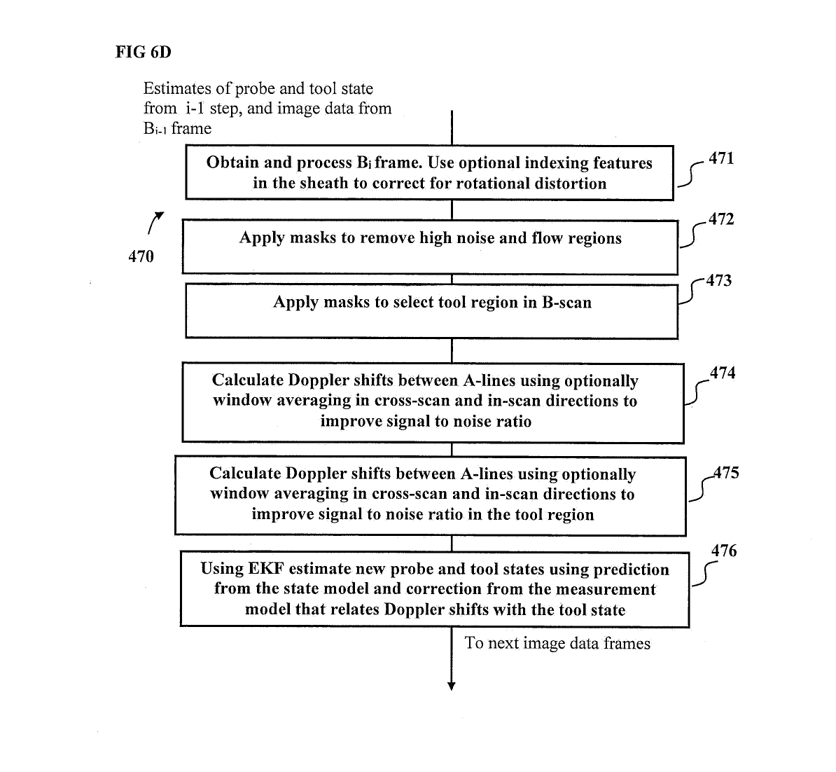

FIG. 6A is a diagram of a 3D image data set illustrating A-lines and B-scan structures. FIGS. 6B and 6C are signal processing flowcharts for calculating a tool position using Doppler signal processing of B-frames and A-lines, respectively. FIG. 6D is a signal processing flowchart for calculating tool position when the a tool and a probe are configured to be repositionable with respect to each other;

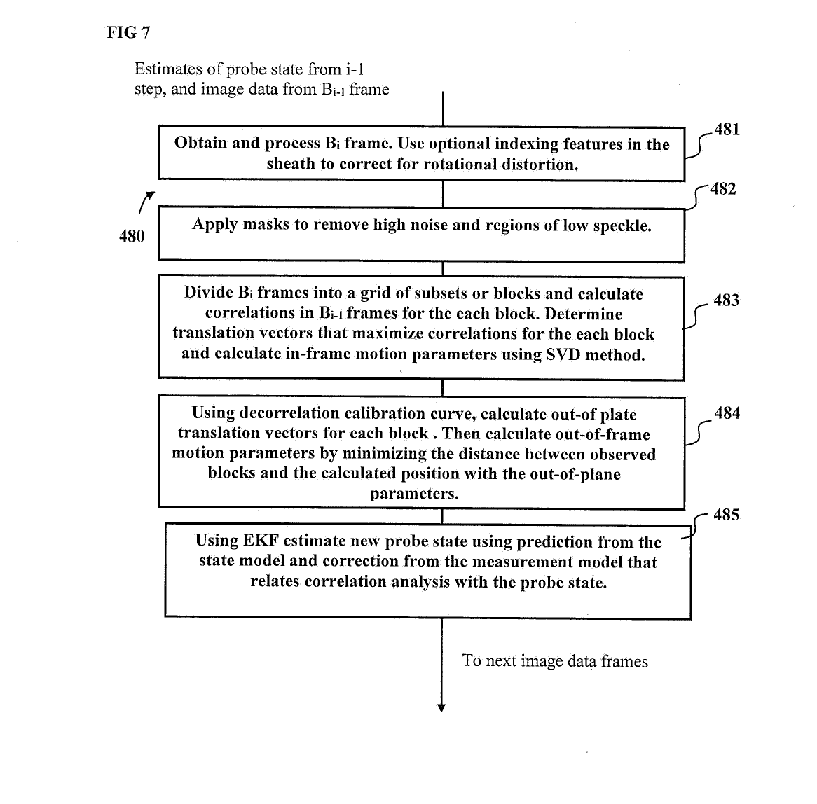

FIG. 7 is a signal processing flowchart for calculating tool position using speckle correlation analysis;

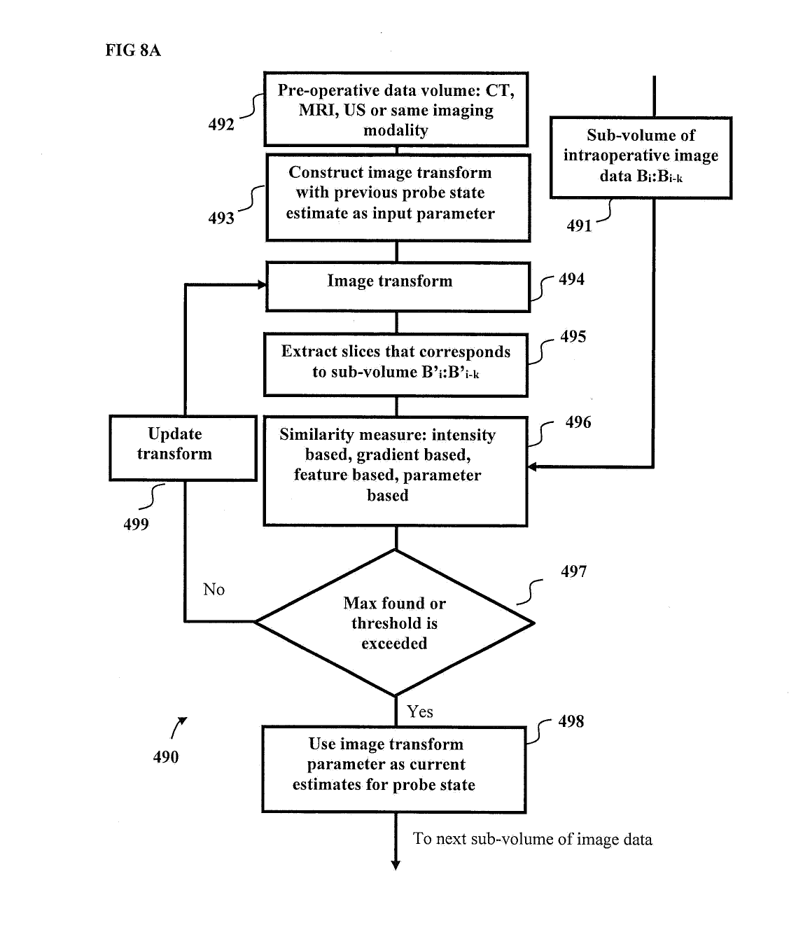

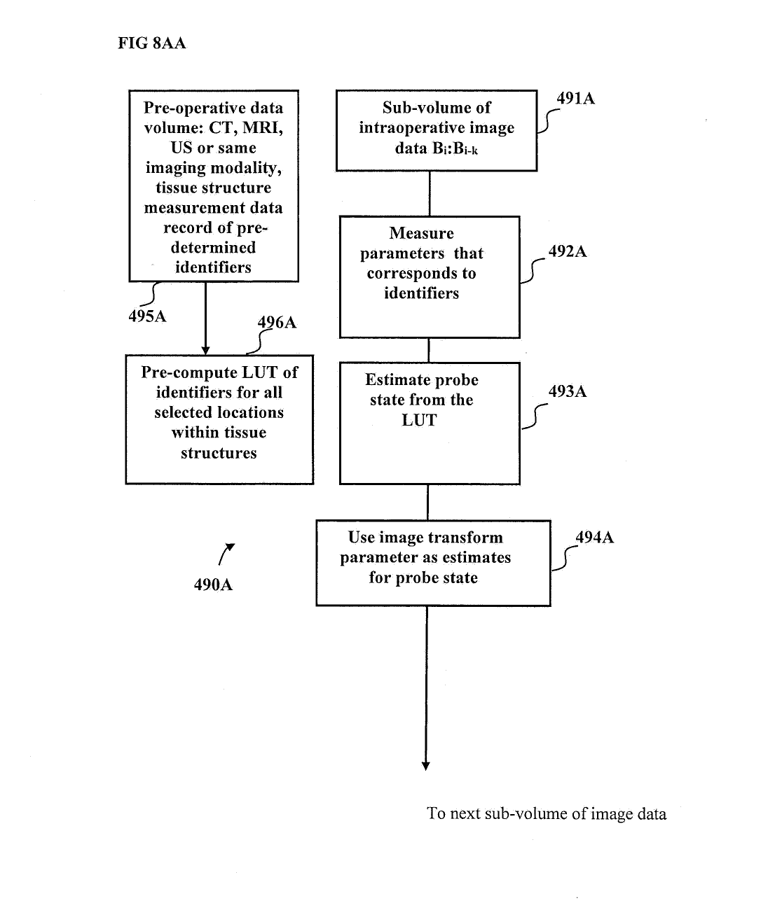

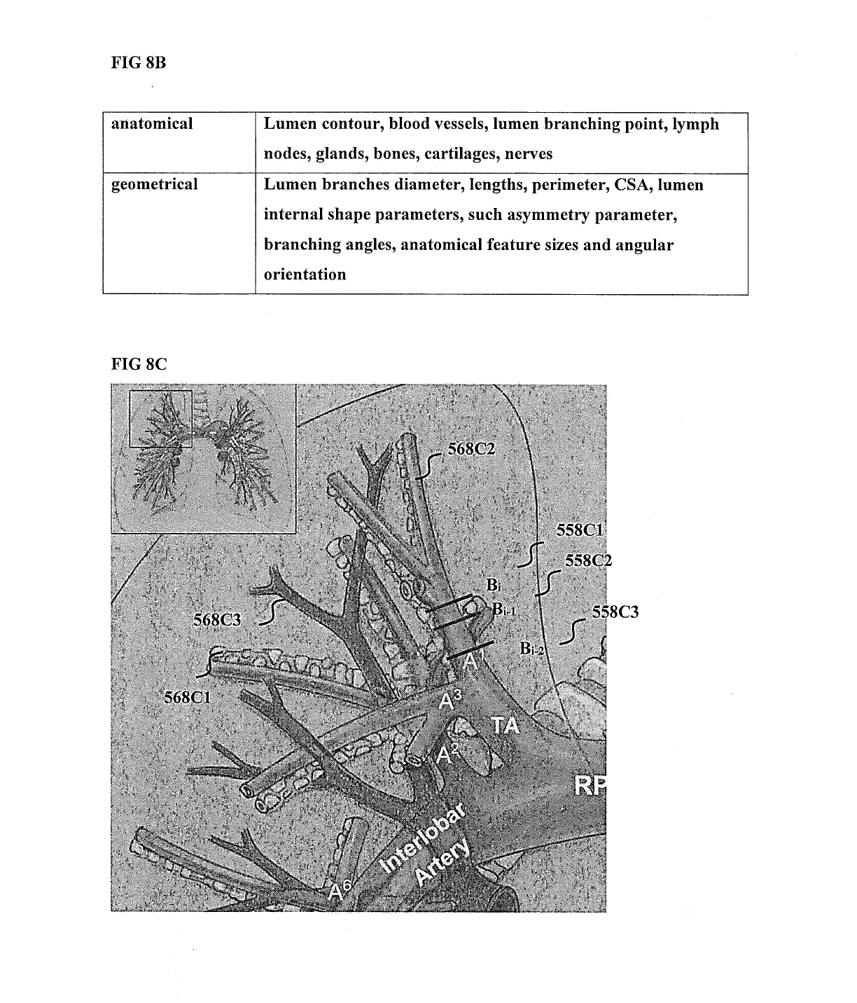

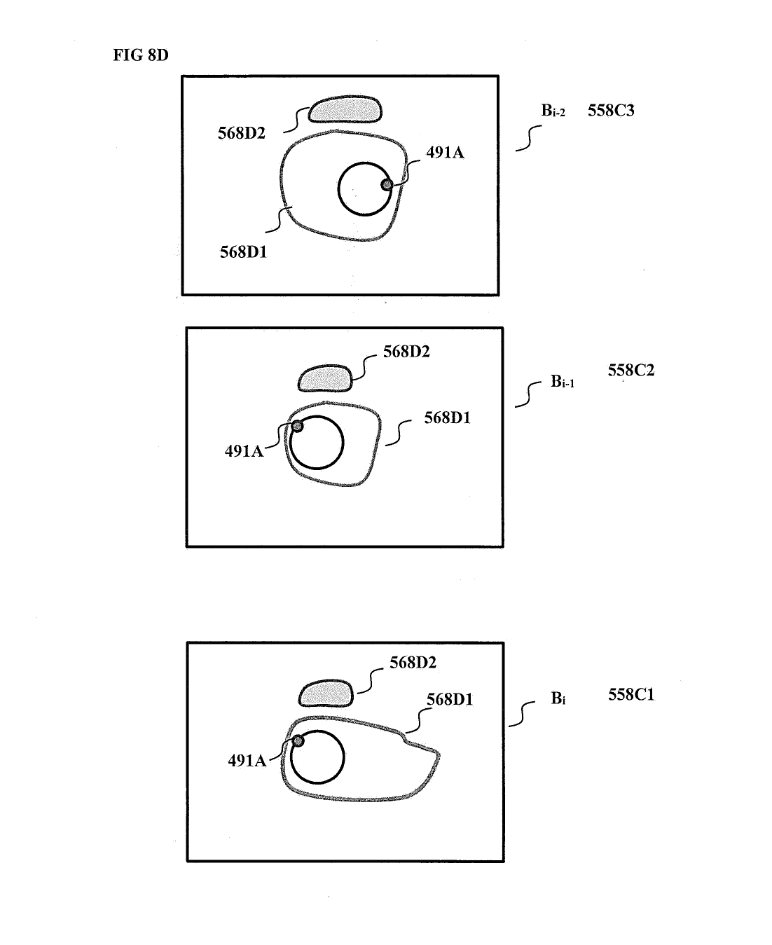

FIGS. 8A, 8AA, 8B, 8C, and 8D describe methods and associated structures that allow determining a position and a pose of a probe during a procedure using pre-procedural image data of various modalities;

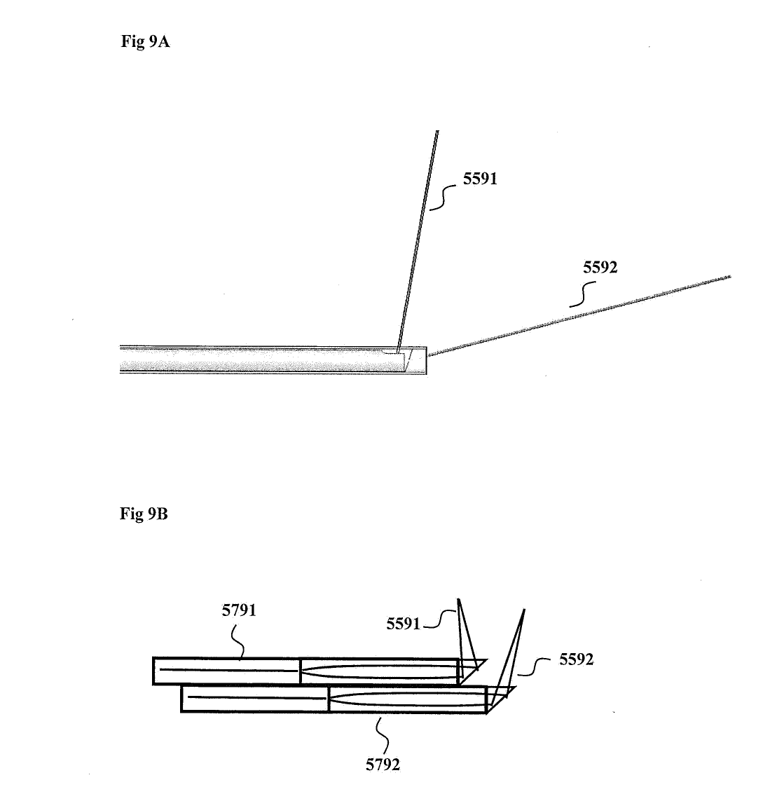

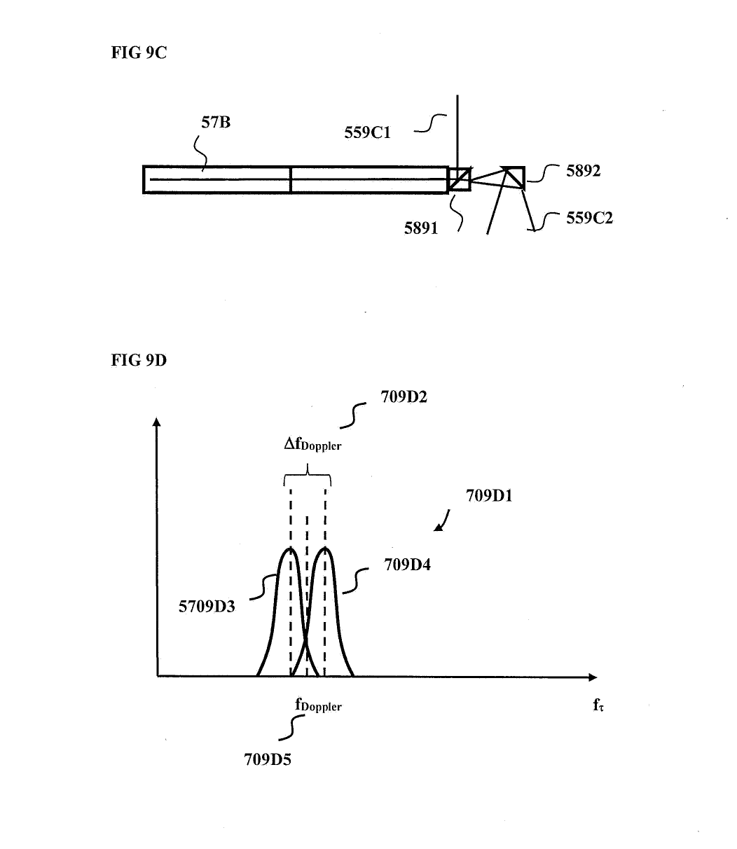

FIGS. 9A, 9B, and 9C are schematic diagrams of dual-beam imaging probes of some embodiments that allow for improved accuracy and robustness of position calculations. FIG. 9D illustrates an example of dual direction signal processing substantially immune to position calculation errors caused by tissue deformation and tissue motion;

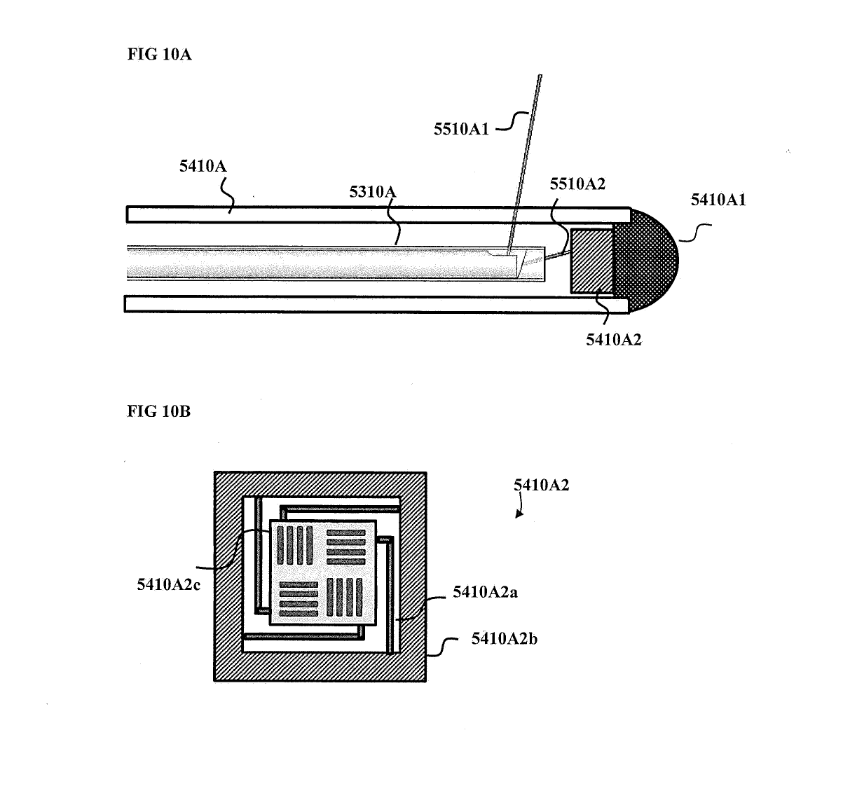

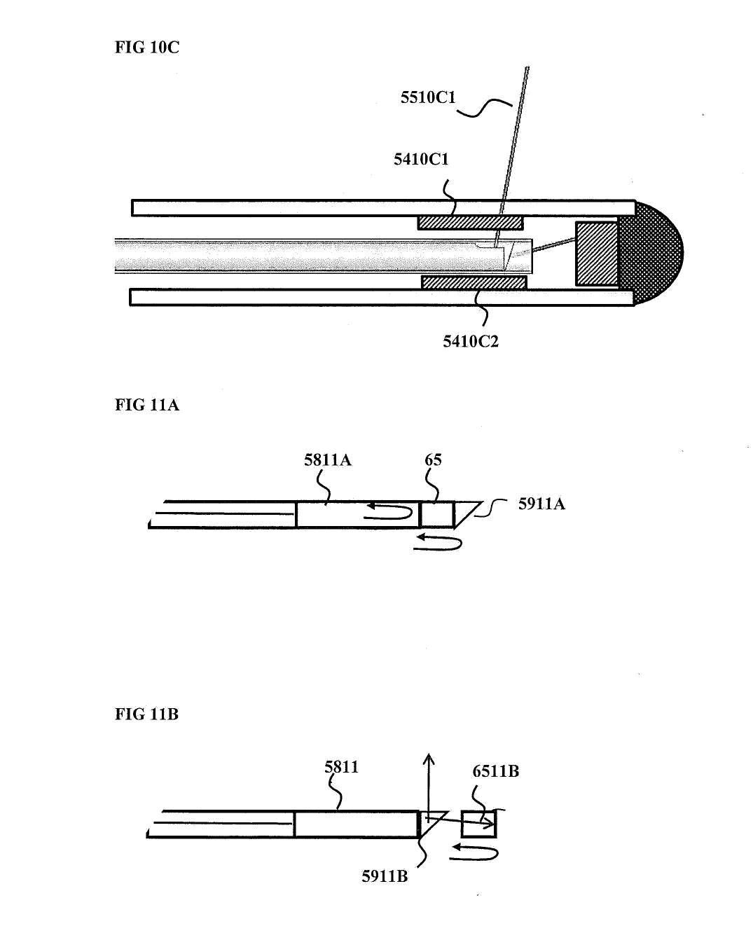

FIGS. 10A, 10B, and 10C are schematic illustrations of probes configured to measure acceleration of the probe distal end, thereby allowing tool position calculations integrated with the acceleration measurements;

FIGS. 11A and 11B are schematic views of probe distal ends configured to measure position changes relative to external electro-magnetic fields, where FIG. 11A exemplifies an arrangement with a magneto-optical element and FIG. 11B exemplifies an arrangement with magneto-strictive element;

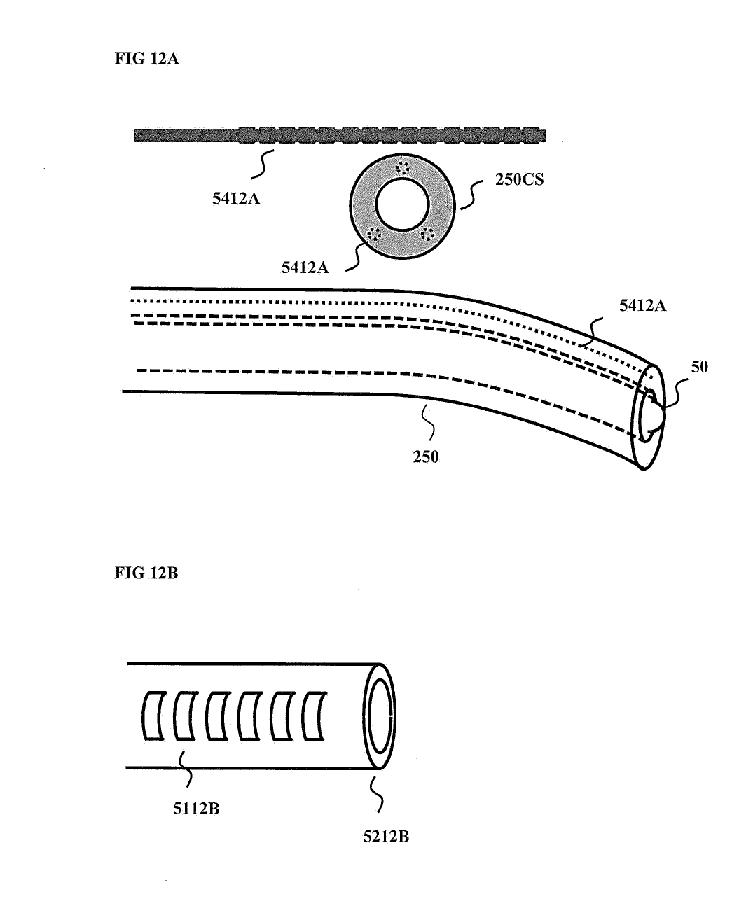

FIGS. 12A and 12B illustrate distal end arrangements capable of bending, thereby allowing tool position calculations using image data of the bended states;

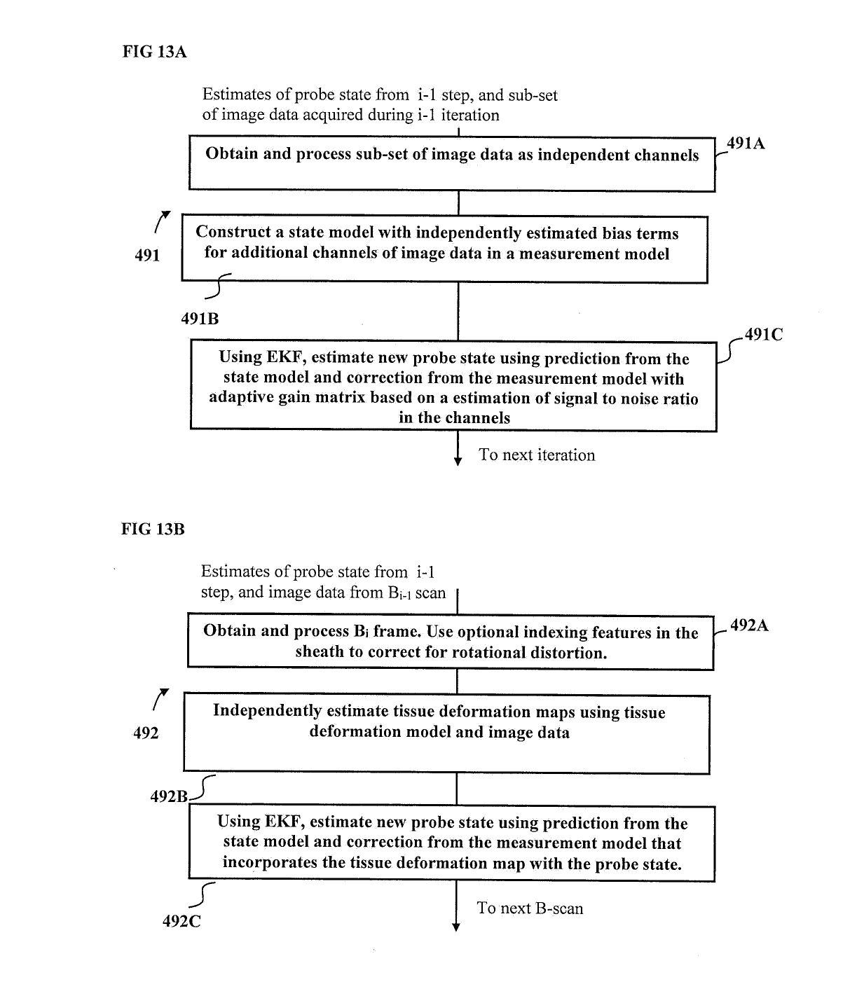

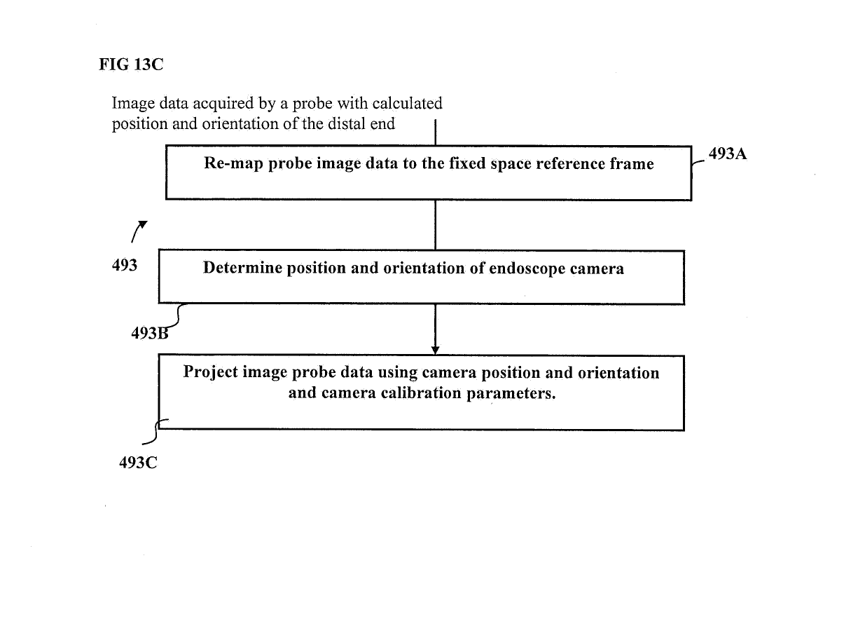

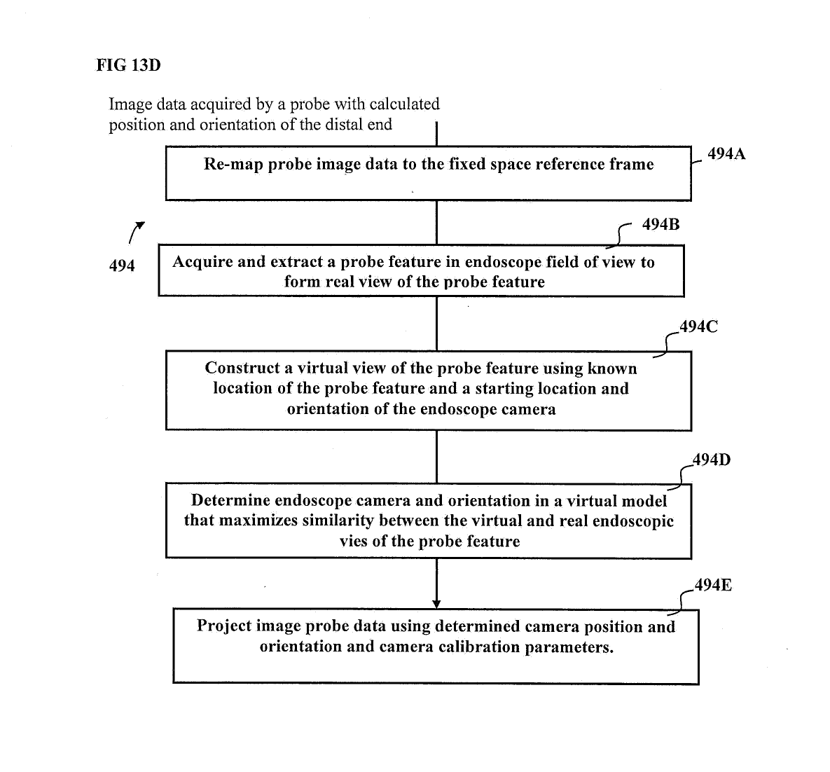

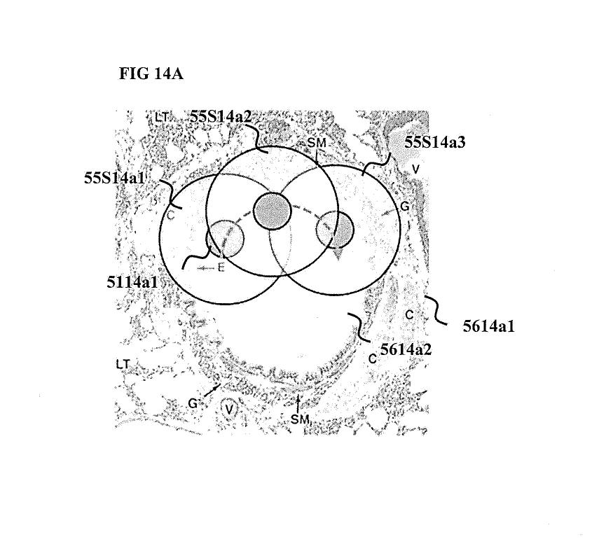

FIGS. 13A, 13B, 13C, and 13D are flowcharts depicting position calculations using fusion of different channels of data, estimation of tissue deformation, and augmenting images of external devices, respectively;

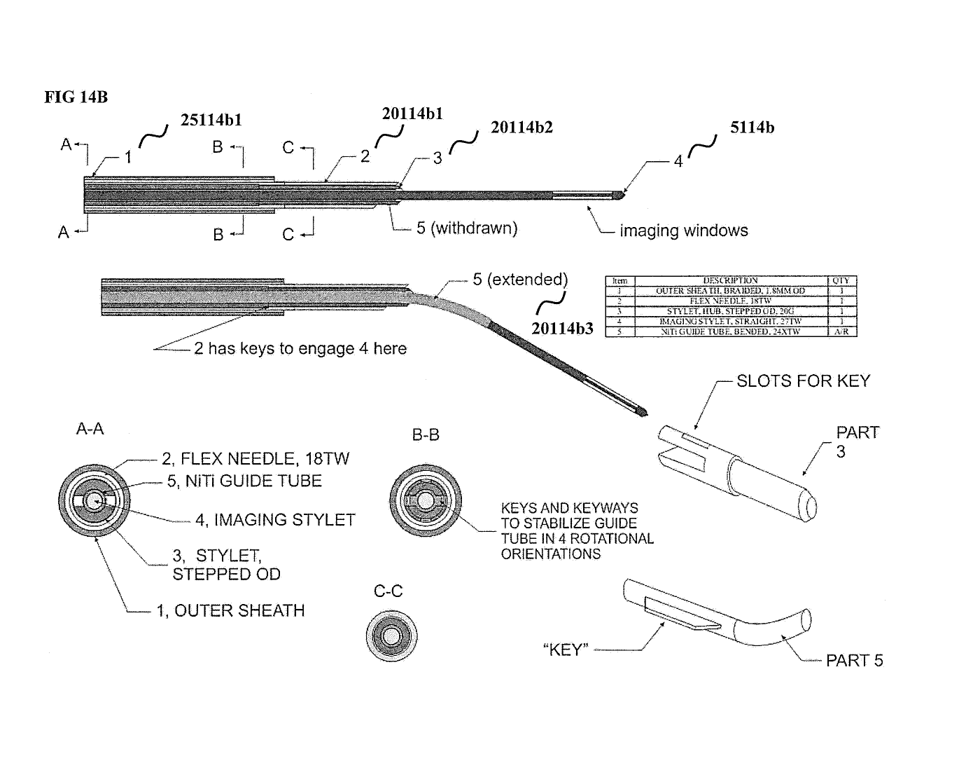

FIGS. 11A and 14B illustrate methods and structures of embodiments capable of an extended field of view during intraoperative imaging;

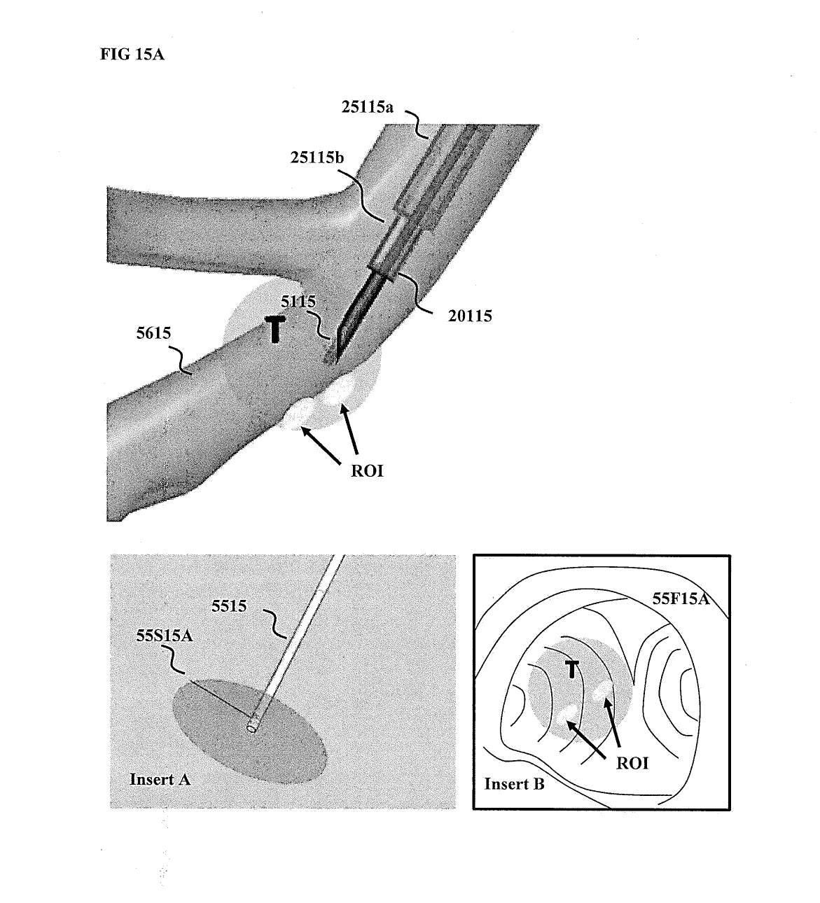

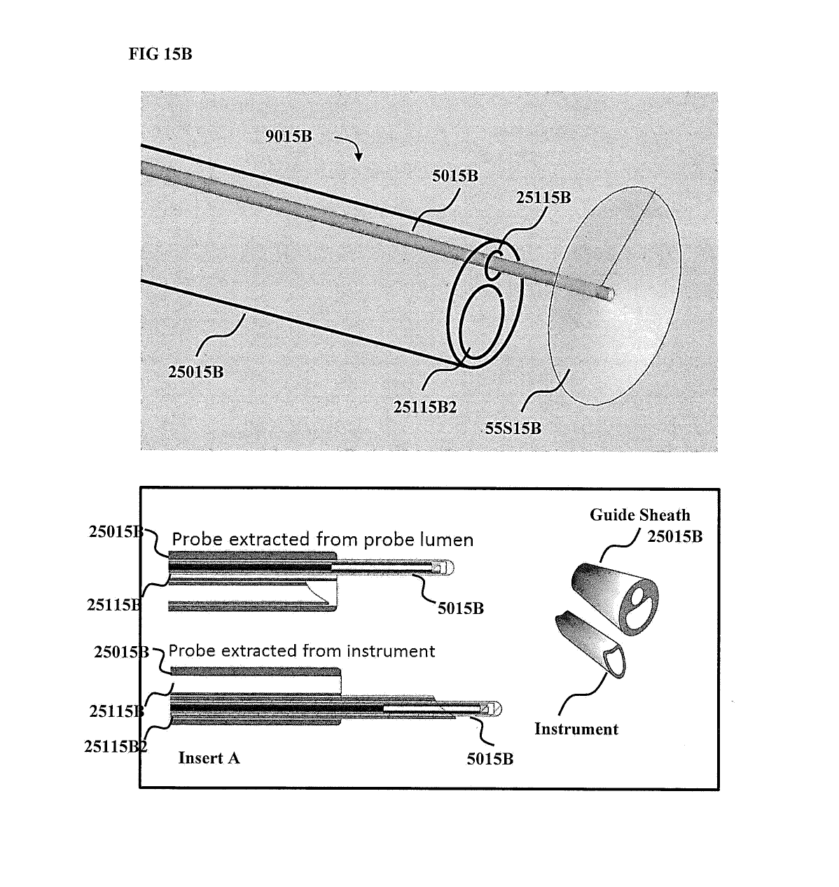

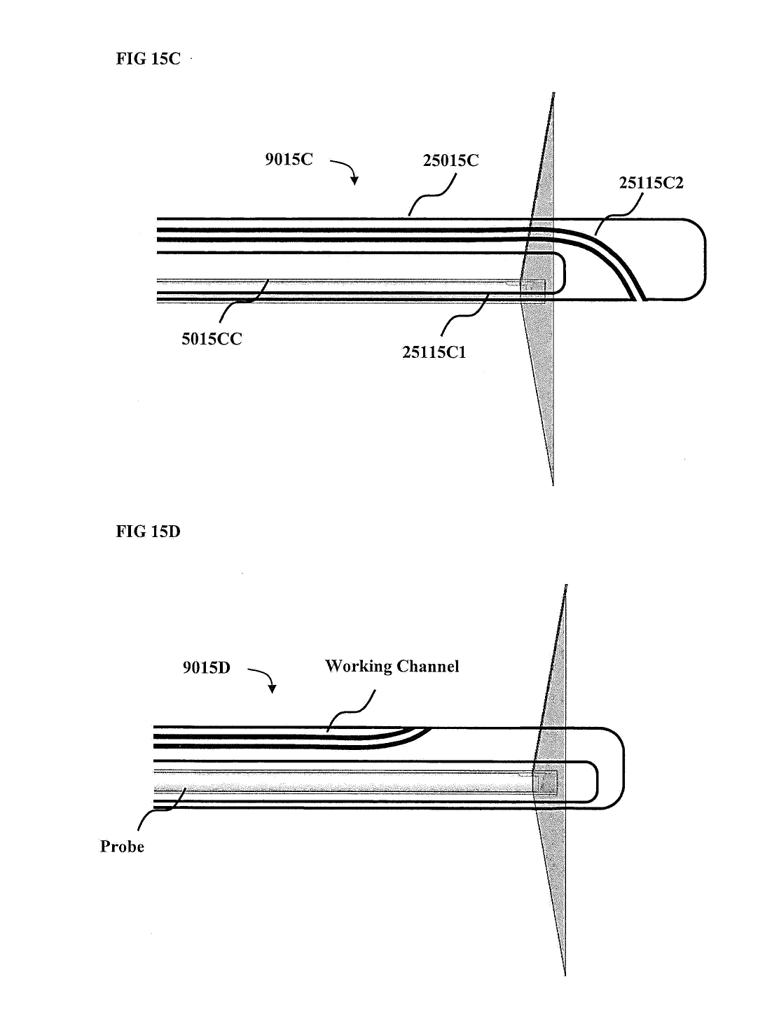

FIGS. 15A, 15B, 15C, and 15D illustrate probe configurations with improved forward direction guidance and control and 3D guidance and control;

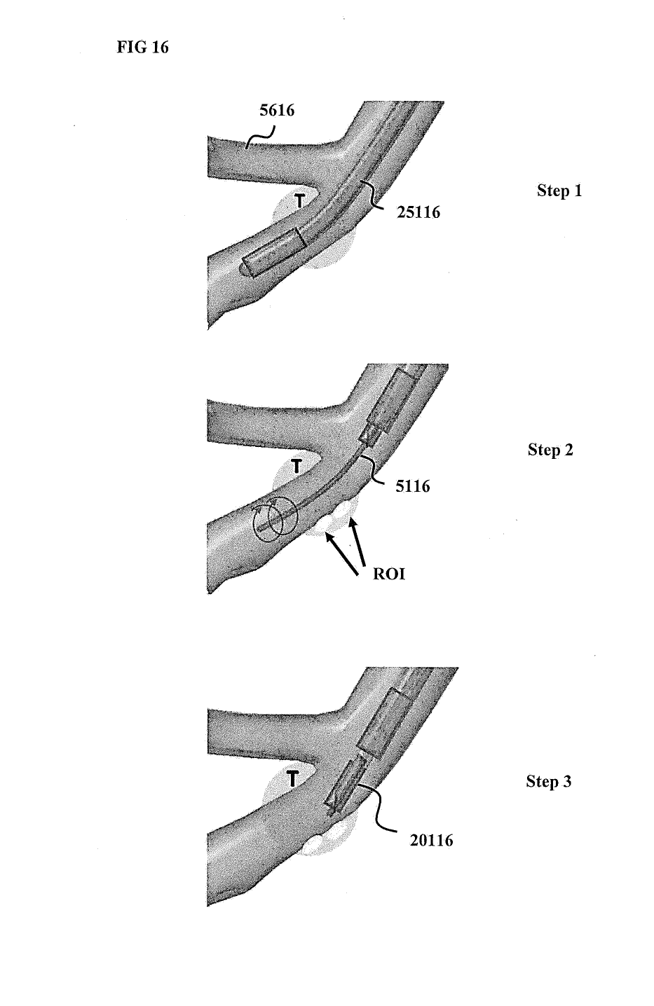

FIG. 16 shows probe positioning during steps of refining and augmenting a pre-operative image data using an intra-operative imaging data obtained with a probe, according to some embodiments;

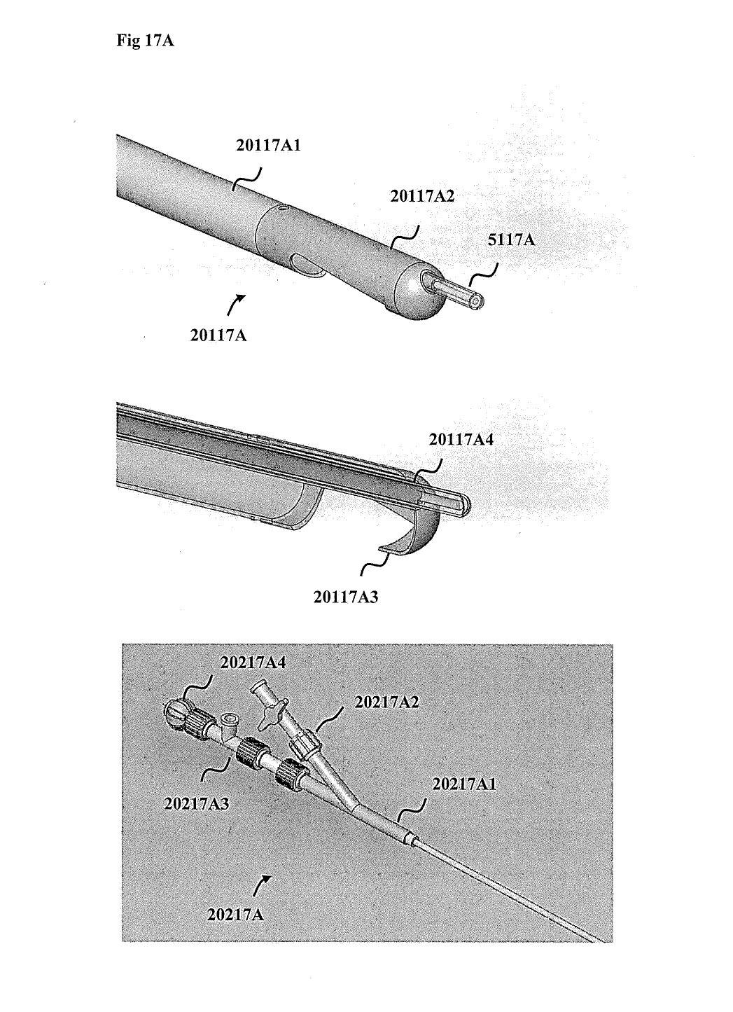

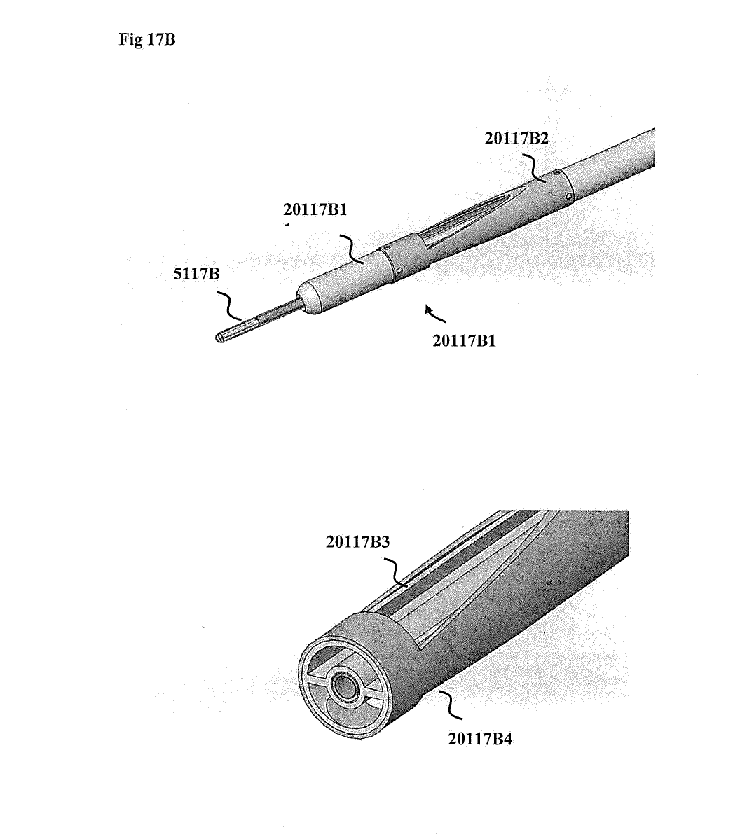

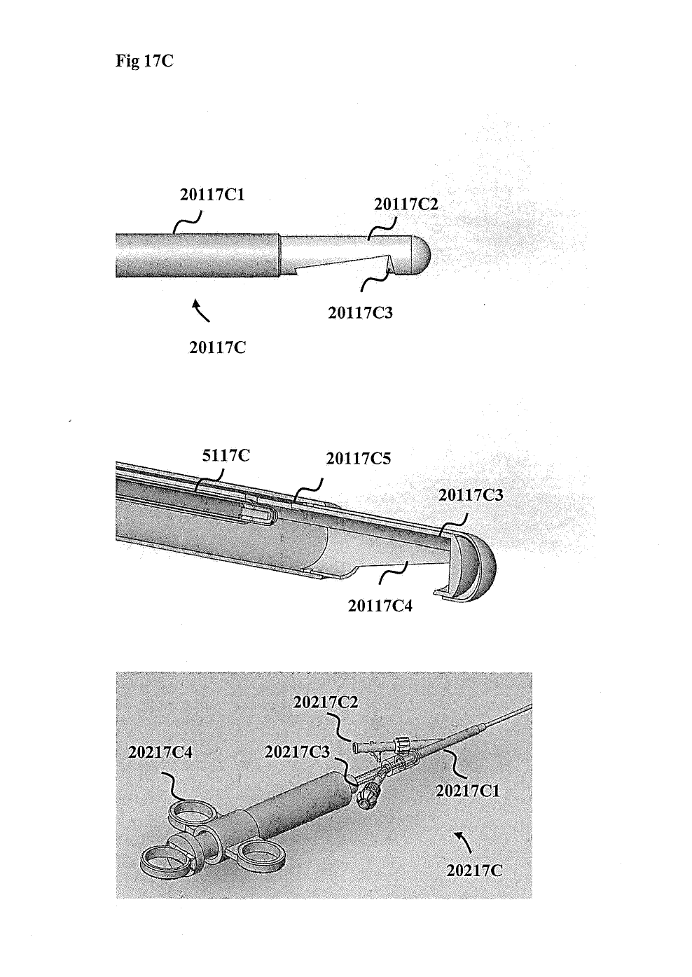

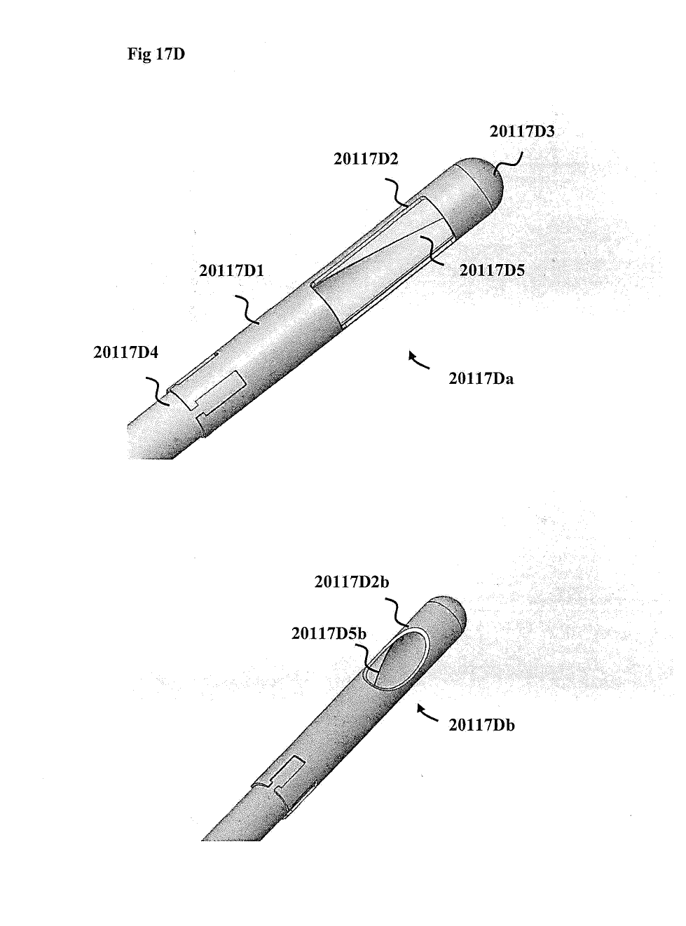

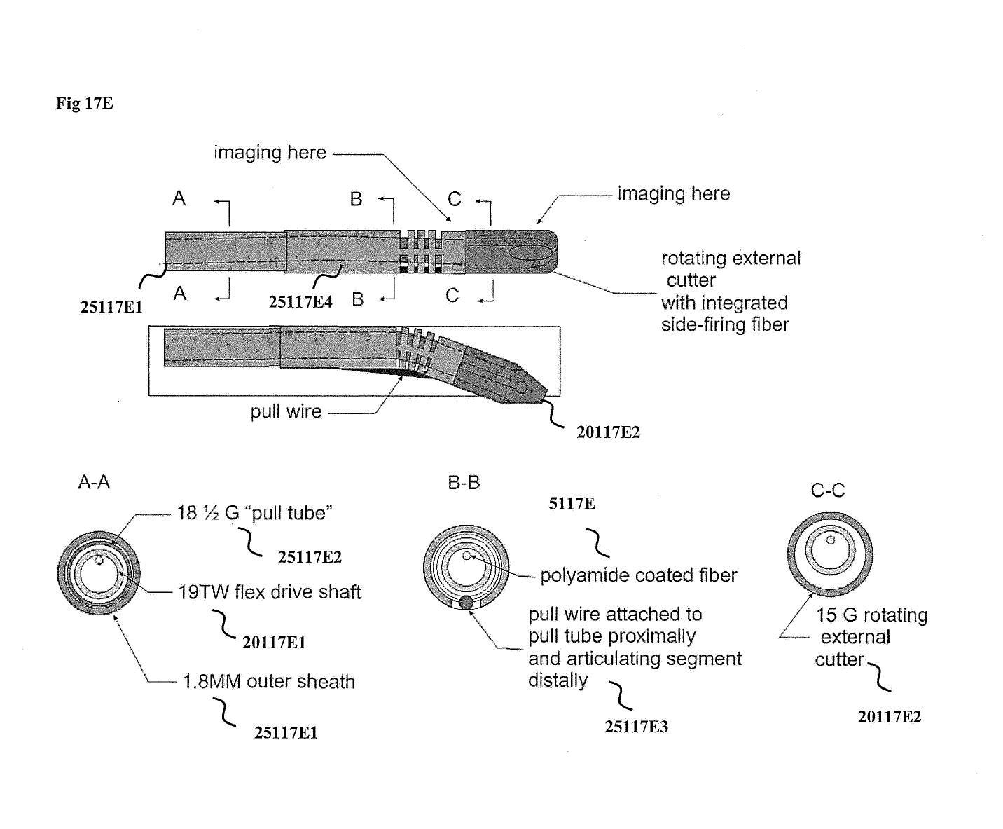

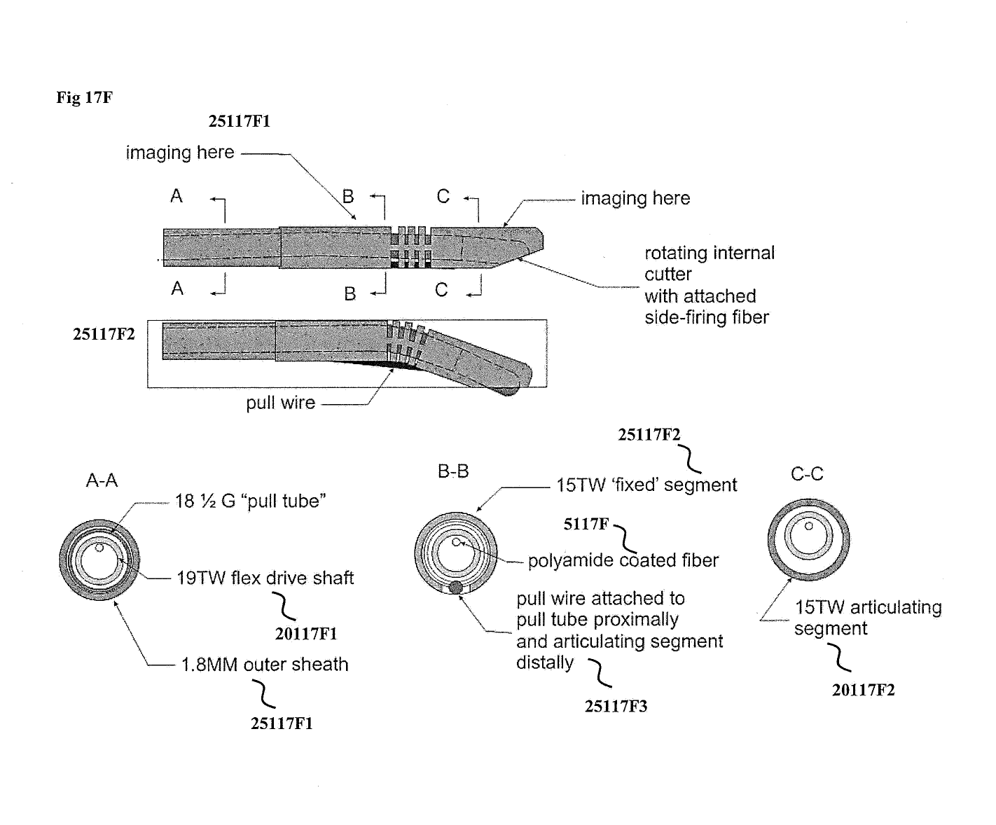

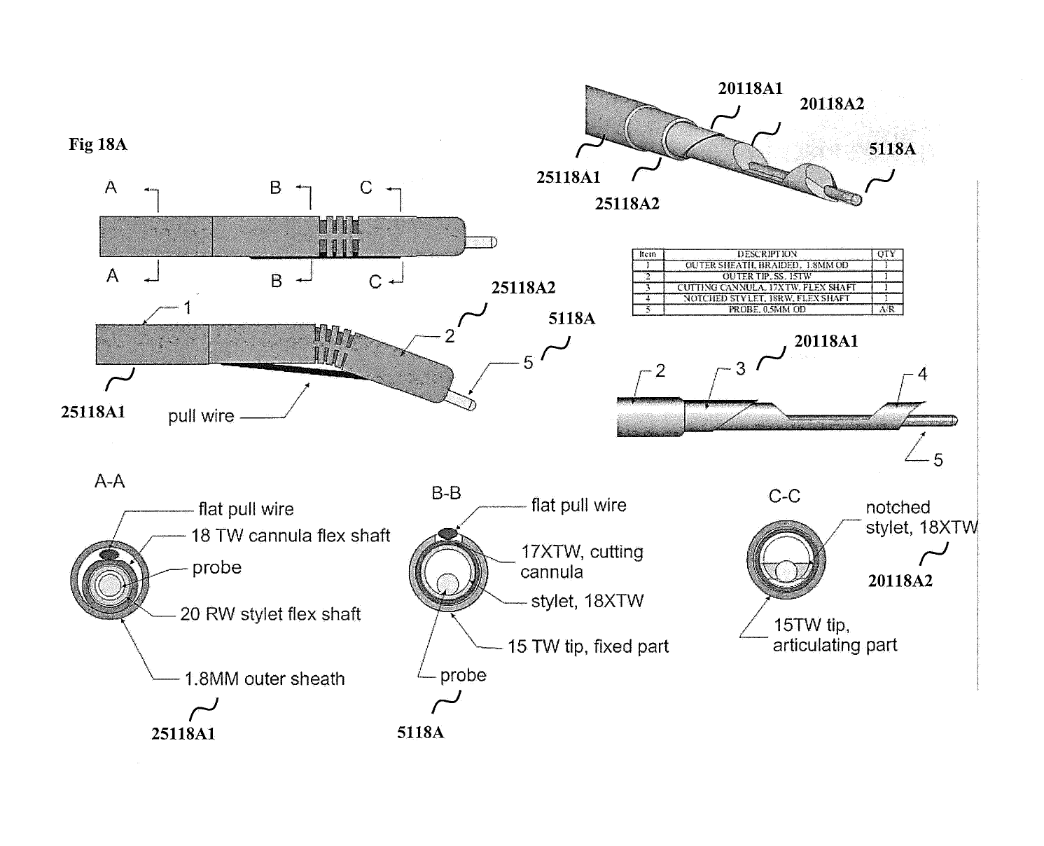

FIGS. 17A, 17B, 17C, 17D, 17E, and 17F illustrate various views of probes having improved alignment for biopsy tools;

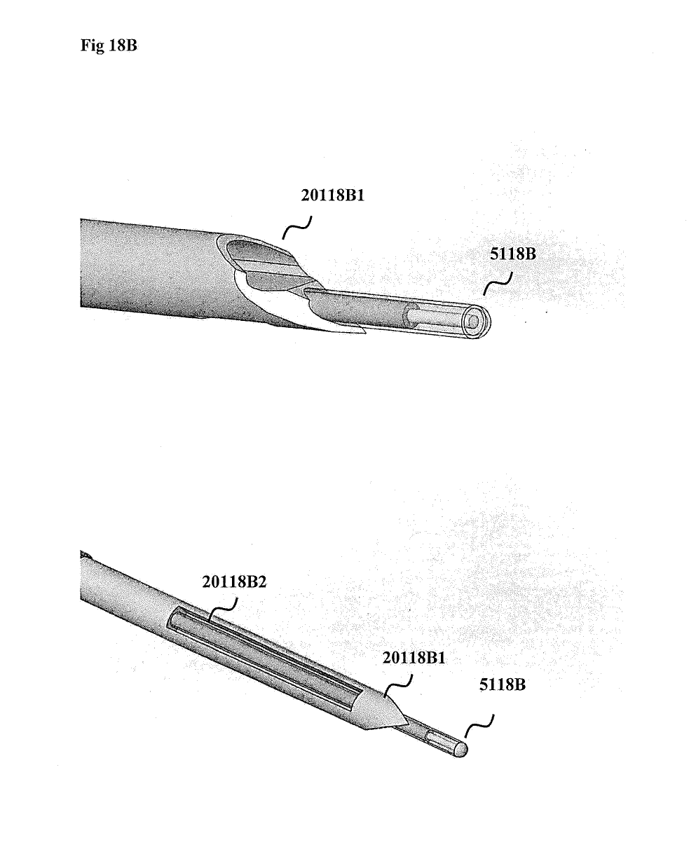

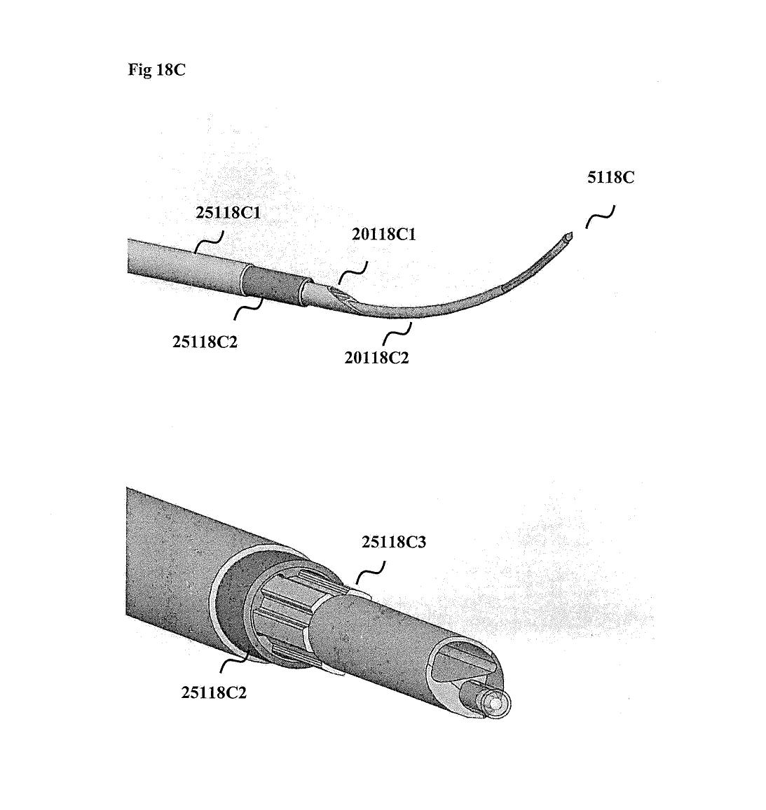

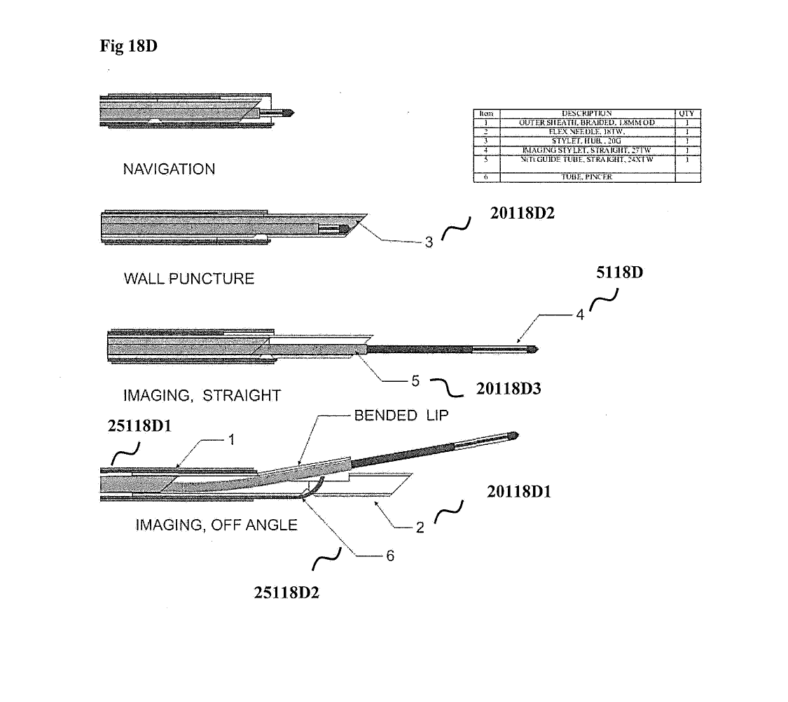

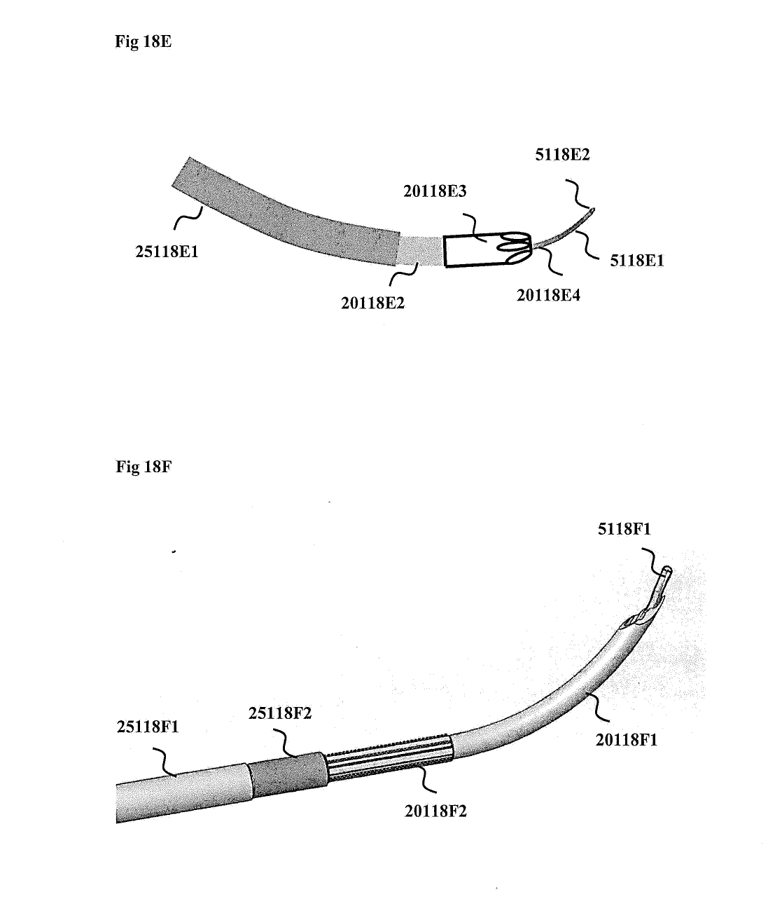

FIGS. 18A, 18B, 18C, 18D, 18E, and 18F illustrate various views of additional probes having improved alignment for biopsy tools;

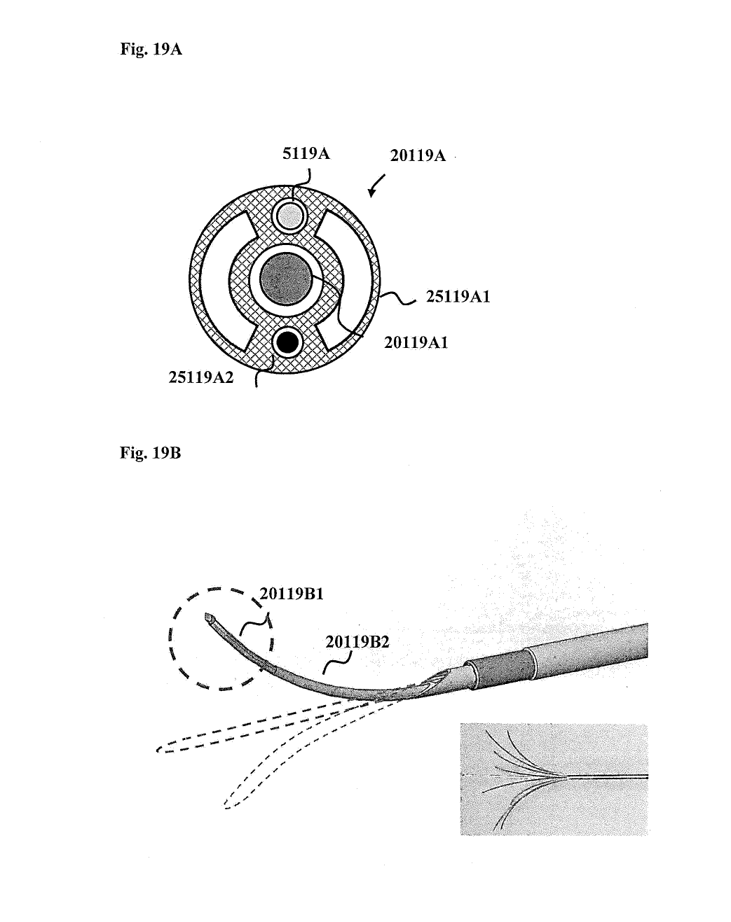

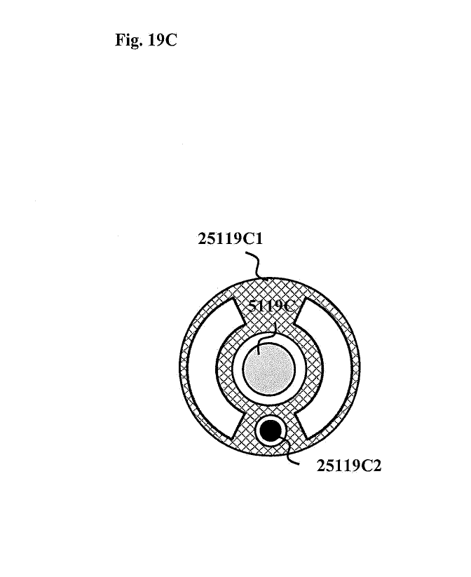

FIGS. 19A, 19B, and 19C show various views of probes with, improved alignment capabilities for ablation and treatment tools;

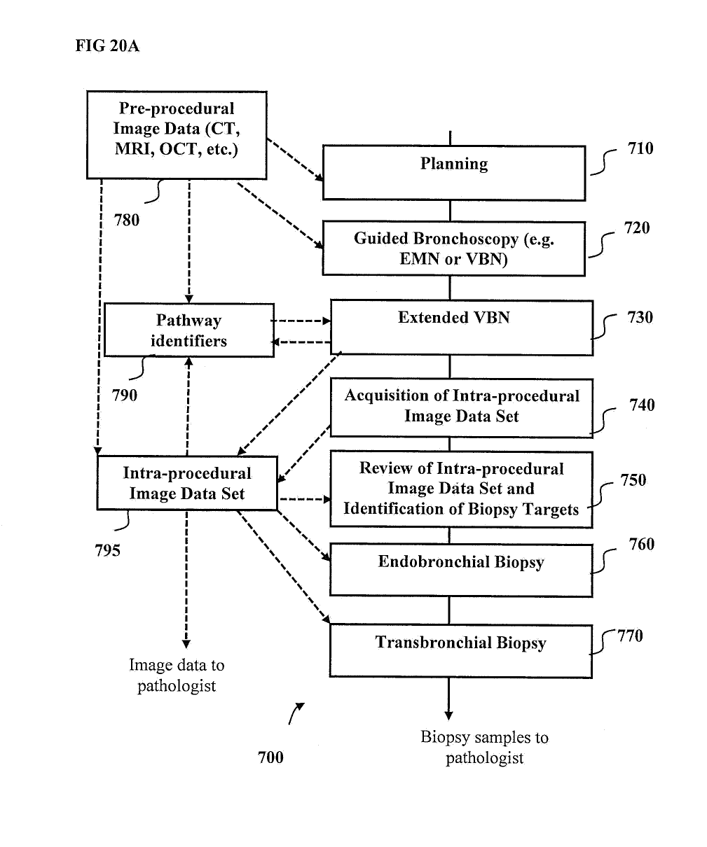

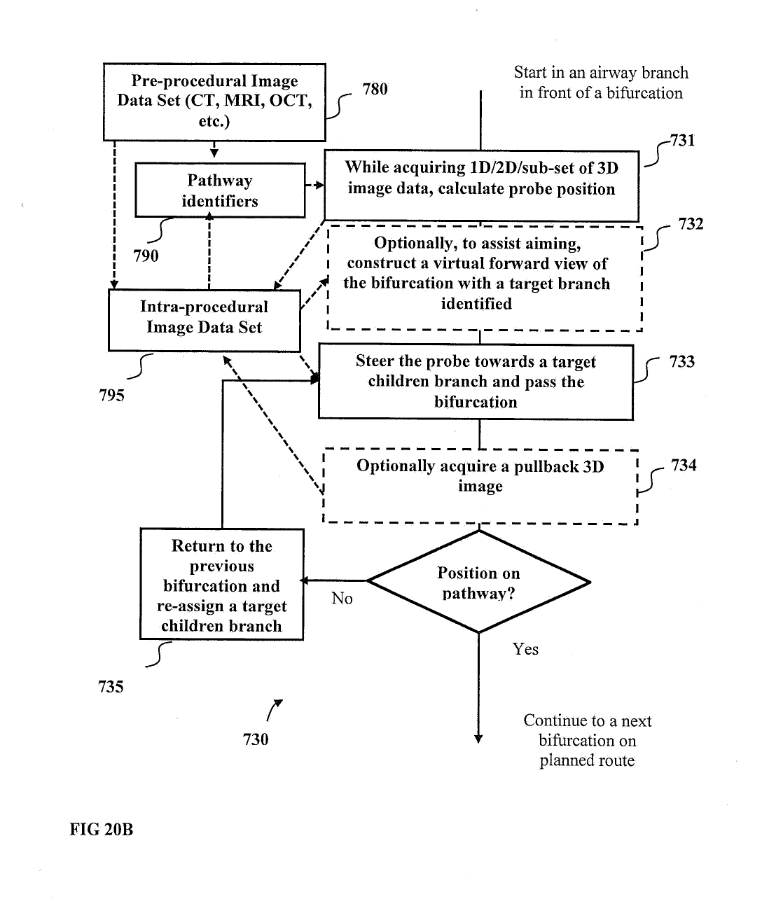

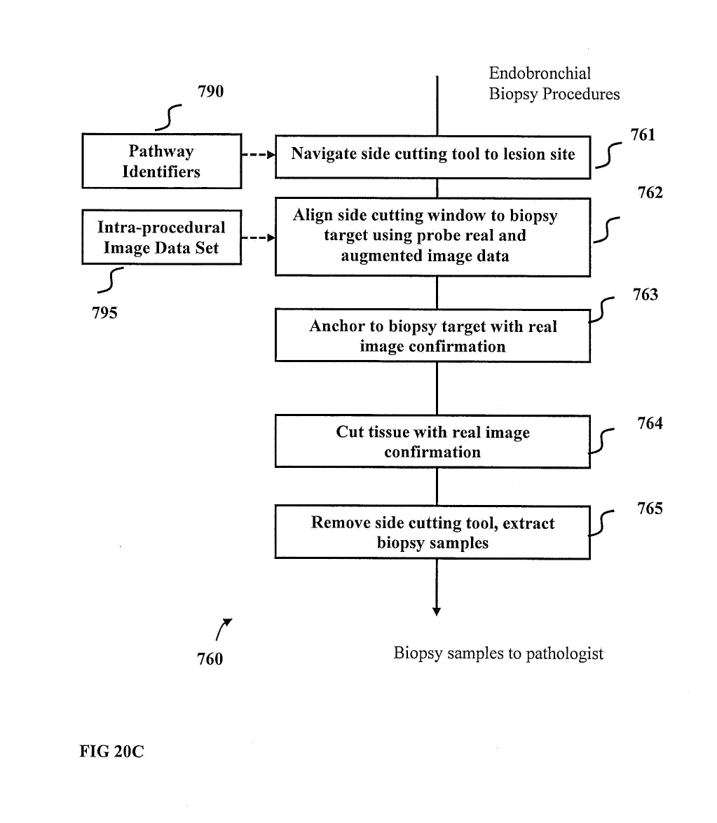

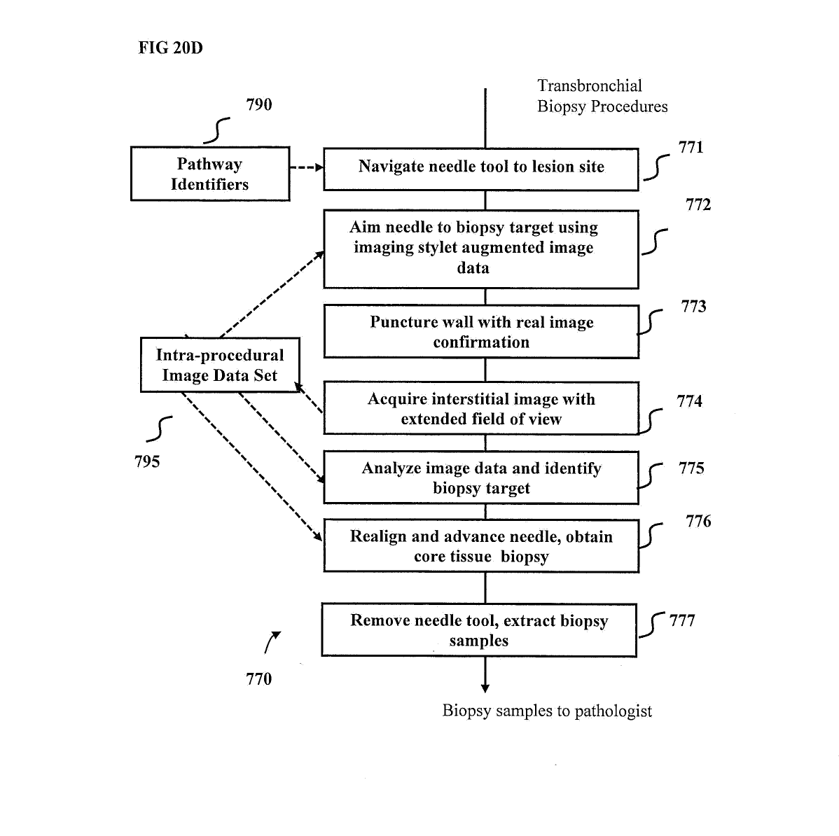

FIGS. 20A, 20B, and 20C are flowcharts of a medical procedure rising an image-guided system and method of some embodiments of the invention;

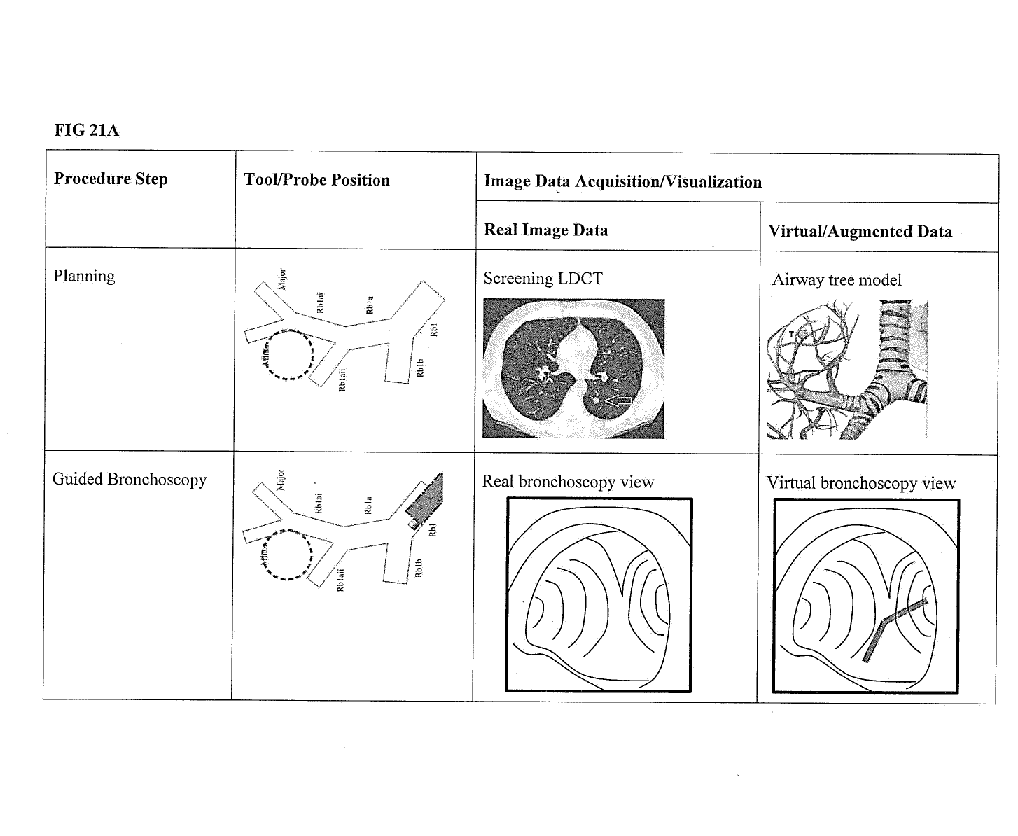

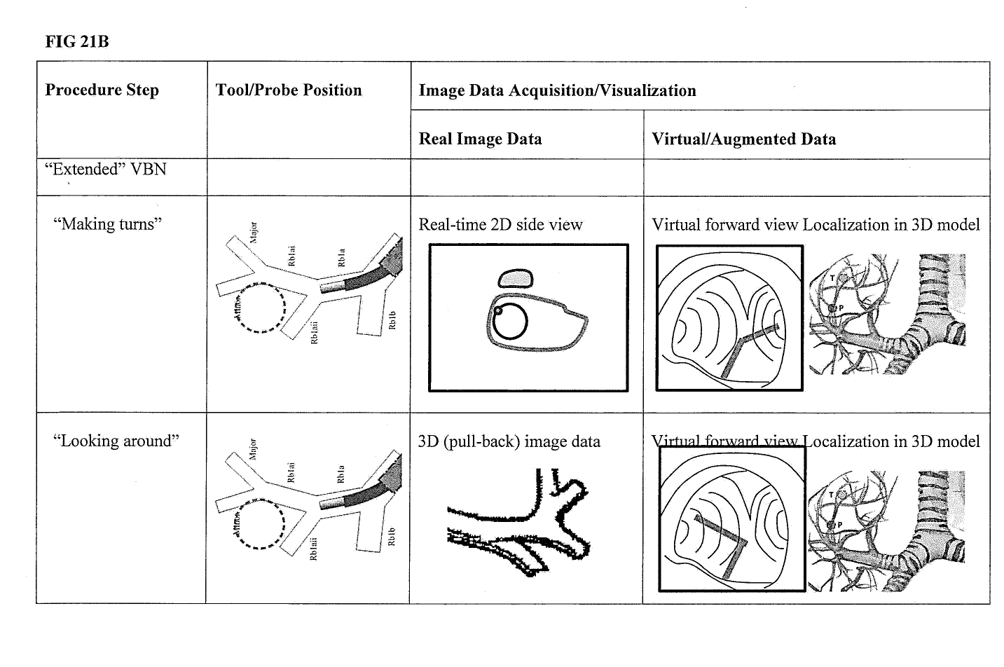

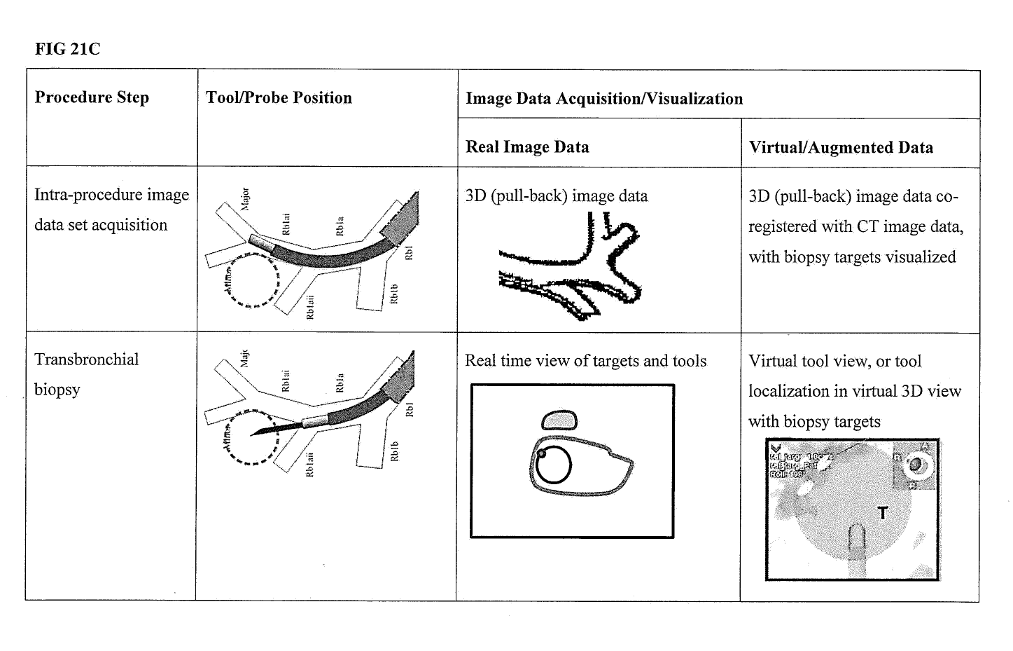

FIGS. 21A, 21B, and 21C are charts showing relationships between image data used to determine positions of devices used in the medical procedures of FIGS. 20A, 20B, and 20C and corresponding virtual or augmented 3D and 2D views that facilitate image guidance using methods and systems of some embodiments of the invention;

FIGS. 22A and 22B are flowcharts of another medical procedure using an image-guided system and method of some embodiments of the invention;

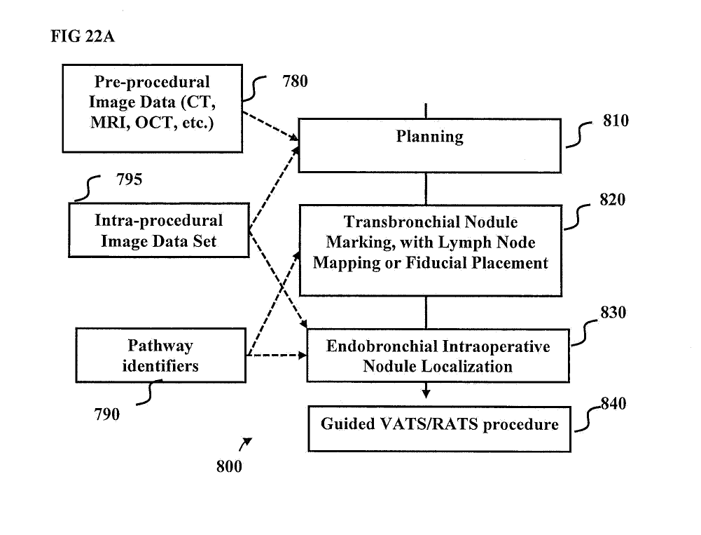

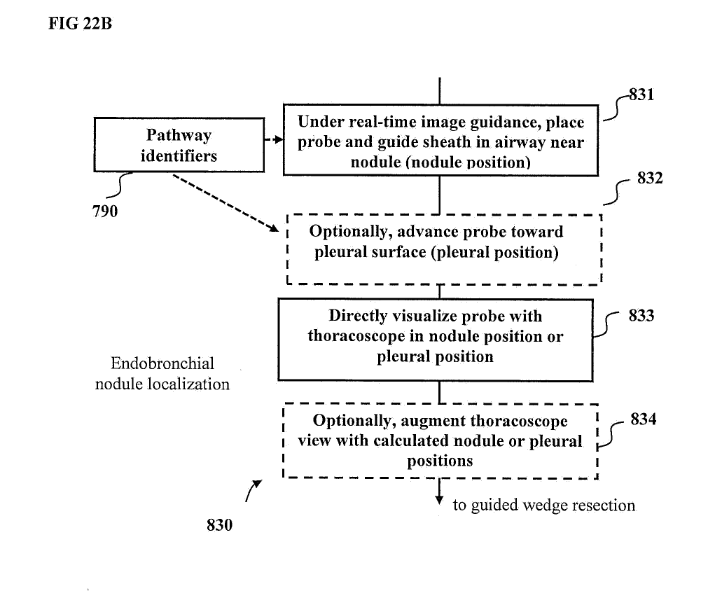

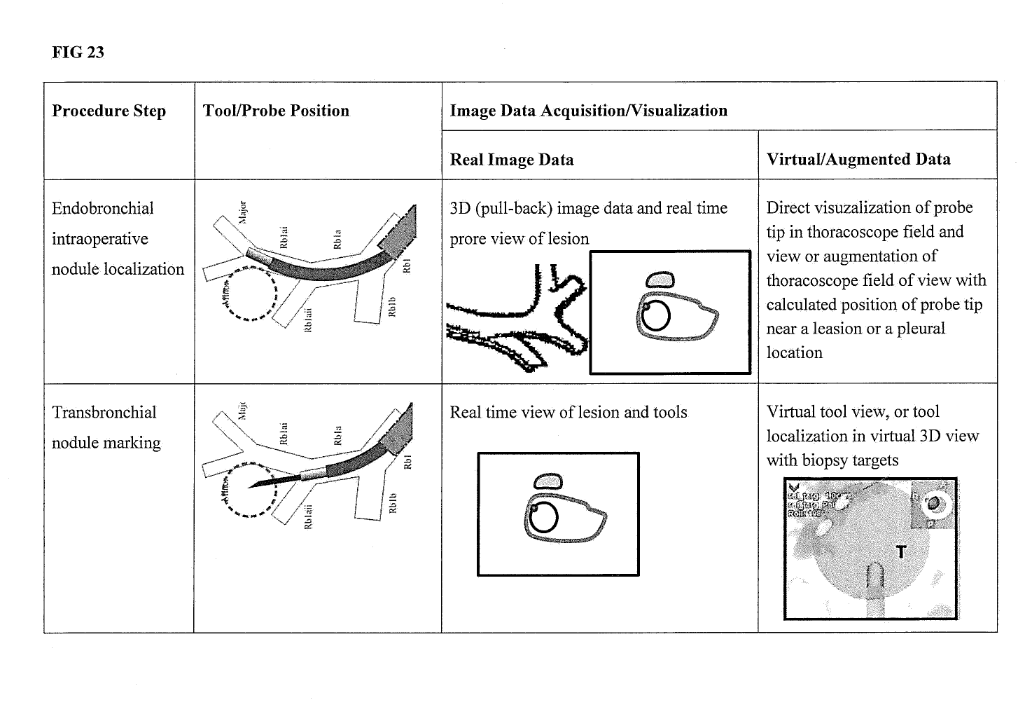

FIG. 23 is a chart showing relationships between image data used to determine positions of devices used in the medical procedures of FIGS. 22A-22B and corresponding virtual or augmented 3D and 2D views that facilitate image guidance using methods and systems of some embodiments of the invention; and

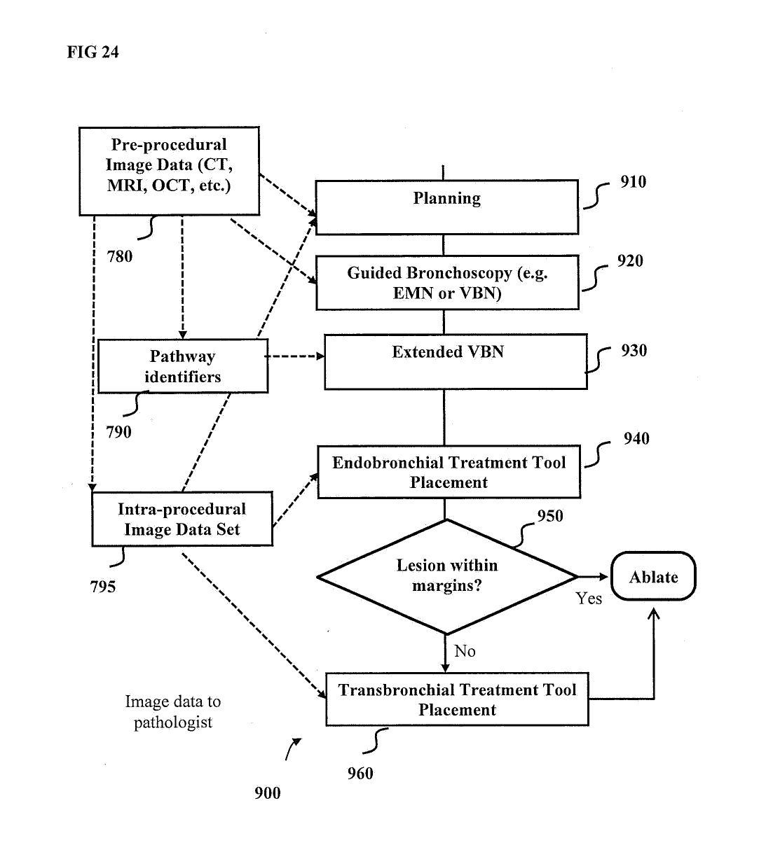

FIG. 24 is a flowchart of yet another medical procedure using an image-guided system and method of some embodiments of the invention.

### DETAILED DESCRIPTION

For clarity of presentation, the following disclosure is structured as follows. The disclosure associated with FIGS. 1-5 is a general description of a medical apparatus and methods of embodiments the present invention. FIGS. 6-13 relate to position calculation aspects of embodiments of the present invention. Embodiments of a medical apparatus characterized by extended field of view imaging, including improved forward view imaging, are described in relation with associated distal end components in reference to FIGS. 14-16. In reference to FIGS. 17-19, several embodiments of an apparatus and associated methods providing improved medical tool placement and alignment to targets are described. Finally, FIGS. 20-24 describe example medical procedures using systems and methods of embodiments of the present invention.

In the context of the present disclosure, the term “distal end” implies a distal end portion of a medical instrument component intended to be placed inside or in close proximity to patient body lumens, cavities, tissue, and other medical procedure targets. The term “proximal end” implies a corresponding opposite portion of the medical instrument component that is intended to be held and/or manipulated by an operator of a medical system or to be interfaced with a medical system. The term “medical tool” implies any interventional diagnostic, treatment, or marking medical instrument or medical device. Non-limiting examples of medical tools within the scope of the present disclosure include: an injection needle; an aspiration needle; a core biopsy needle; side cutting needles; biopsy or cutting forceps; snatches; fiducial placement devices; stent placement devices; balloons; surgical cutters; RF, MW, laser, cryogenic biopsy or ablation devices; photodynamic therapy delivery devices; brachytherapy delivery devices; localized drug delivery devices.

Additionally, in the context of the present disclosure, the terms “position,” “probe position,” and “tool position” imply position of a probe and/or associated medical tools, unless the context clearly dictates otherwise. The term “position” may also imply both a position and angular orientation of a device or imaging probe distal end, unless the context clearly dictates otherwise. The term “position” may further imply a global position within a patient body relative to a tissue target, unless the context clearly dictates otherwise. The term “global” implies located beyond the local field of view of an imaging probe. The term “intra-operative data” and related terms imply image data acquired by an imaging probe, while the term “pre-operative data” and related terms imply either image data acquired by other imaging devices or image data acquired by an imaging probe in a previous medical procedure or both, unless the context clearly dictates otherwise. The term “real-time” implies substantially continuous and sufficiently fast so that an alignment of a probe or a medical tool relative to a target is not lost due to uncontrolled outside motion, tissue motion, or tool exchanges. In addition, the term “or” is an inclusive “or” operator and is equivalent to the term “and/or”, unless the context clearly dictates otherwise.

### Problems Solved by Embodiments of the Invention

In order to provide a 3D intra-operative image guidance for medical procedures without the computational burden, high cost, and high complexity of acquiring, processing, and rendering 3D data in real time, some embodiments of the invention provide a tool handling arrangement in a form of a guide sheath with an imaging probe in communication with a system console and configured to acquire 3D image data intra-operatively. The guide sheath may also include a medical tool configured to perform a therapeutic, diagnostic, surgical, or tissue marking procedures. The system console is configured to calculate, in real time, a position of the tool relative to a target identified within the intra-operative data set. Tool position is calculated using 1D, 2D, or 3D sub-sets of 3D image data acquired by the imaging probe in real-time. The system console is also configured to render a 3D scene containing the tool and the 3D image data with the target identified. The system console is further configured to process a pre-operative, reference image data with the intra-operative data to improve target identification. In some embodiments, calculation of the tool position is based on Doppler signal processing, correlation analysis of image speckles, and/or comparing image data obtained in the intra-operative and the pre-operative 3D data set with image data obtained in the 1D, 2D, or 3D sub-sets of 3D data. By using 1D, 2D, or 3D sub-sets, some embodiments can enable faster processing while still providing 3D information or renderings compared to systems that use full 3D imaging data.

In order to provide 3D images with increased 3D field of view, or range of imaging, while maintaining small insertion widths for minimally-invasive image guidance and control of medical procedures, embodiments are provided with an arrangement of a flexible imaging probe and a guide sheath that are configured to be repositionable or steerable, at the distal end, within a patient body. The system console is configured to calculate a position of the imaging probe during intra-operative 3D imaging using 1D, 2D, or sub-sets of 3D data acquired by the imaging probe in real-time. The system console is further configured to remap the 3D image data using the probe position information to render a 3D scene with an extended field of view.

In order to provide an image-guided system with side-view imaging probes having improved real-time guidance and control in a forward direction, an image-guided system of some embodiments is configured to provide virtual forward views by calculating the position of the imaging probe relative to a target using real-time 1D, 2D, or sub-sets of 3D imaging. The image guided system is also configured to remap and re-render pre-operative 3D image data in accordance with the calculated probe positions. In certain embodiments, the image-guided system is configured to refine or to augment a pre-operative data with the intra-operative data obtained with the imaging probe. In some embodiments, the image guided system is configured to render a 2D or 3D image data with a position of a tool with respect to the target.

In order to provide an image-guided system with improved alignment of medical tools with respect to optimal intervention sites in small spaces of minimally invasive interventional procedures, some embodiments provide a medical tool handling arrangement in a form of a guide sheath configured to contain an imaging probe and a medical tool. An image guided system is configured to directly visualize a distal end of the tool and a target and to calculate a position of the tool distal end relative to the target when the tool distal end and/or the target cannot be directly visualized. In some embodiments, the guide sheath or the imaging probe are configured to be insertable in a medical tool. The guide sheath is configured to be repositionable or steerable within patient body in certain embodiments. In some of the embodiments, artificial or natural features are detected by the imaging probe to determine position of the tool with respect to the target. Biopsy tool embodiments with improved side tissue collection and improved forward tissue collection are also provided.

Methods of using the above-described image-guided systems to address the above objectives are also provided.

### General Description

FIGS. 1A, 1B, and 1C illustrate a distal end of a medical apparatus 150 according to some embodiments of the invention. The medical apparatus 150 can include a tool handling arrangement, or guide sheath, 250, an imaging probe 50, and a medical tool 200. As shown in FIGS. 1A-1C and further described below, the medical tool 200 and the imaging probe 50 may be contained and collectively or individually movable within the guide sheath 250. Furthermore, the guide sheath 250, the medical tool 200, and the imaging probe 50 can be in operable communication at their corresponding proximal ends with a imaging or system console 100, as shown in FIG. 2A. Accordingly, in some embodiments, the imaging probe 50, the medical tool 200, the guide sheath 250, and the console 100 may collectively be referred to as the medical apparatus 150 or a medical system.

In use, the imaging probe 50 may be configured to acquire image data intra-operatively, and the system console 100 may be configured to receive the image data from the imaging probe 50, process the image data, and calculate a position of the medical tool 200 or the imaging probe 50 relative to a target in a patient body using the image data (e.g., via a data-processing unit of the system console 100). More specifically, embodiments of the invention generally include an arrangement of the imaging probe 50 and the medical tool 200 that (i) allows a direct “real” intra-operative visualization of the tool or probe distal end relative to a target within the probe field of view and/or (ii) allows for obtaining information about a position of the tool or probe distal end relative to the target intra-operatively using 1D, 2D, and/or sub-sets of 3D image data acquired in real-time by the imaging probe 50. This position information can then be used by the medical apparatus to render a “virtual” visualization of the tool or target.

With respect to the guide sheath 250, the guide sheath 250 can include an elongated flexible body or shaft 251D1 with a distal end 251 (shown in FIGS. 1A-1C) and a proximal end 252 (shown in FIG. 2A). In some embodiments, for better handling and better maneuverability within patient body lumens, the guide sheath 250 may be braided. However, non-braided guide sheaths may be contemplated within the scope of this disclosure. In some embodiments, the flexible shaft 251D1 may be a substantially flexible tube made of metal, a metal alloy, or a different material, and may include one or more internal lumens, channels, or slots configured to accept the imaging probe 50 and/or the medical tool 200. The flexible shaft 251D1 may also be slotted or patterned to improve torqueability or flexibility, as shown in FIGS. 4B and 4C. Alternatively, the guide sheath 250 (and/or the imaging probe 50 or the medical tool 200) can be at least partially made of solid metal or alloy tubes, for example in applications that require less flexibility. The solid or slotted metal tubes can also be overcoated with a plastic material (not shown) or can have internal plastic liners.

With respect to the lumen(s) of the guide sheath 250, in some embodiments, as shown in FIG. 1A, the guide sheath 250 includes a single lumen, and the medical tool 200 and the imaging probe 50 may be contained within the single lumen in a substantially co-axially nested arrangement. In such embodiments, the medical tool 200 can be equipped with a lumen or a working channel sized to accommodate the imaging probe 50. In other embodiments, as shown in FIG. 1B, the guide sheath 250 includes more than one lumen, where the medical tool 200 and the imaging probe 50 may be contained within their own respective lumens in a laterally offset arrangement. In such embodiments, the medical tool 200 need not include a working channel or lumen dedicated to the imaging probe 50. While only two lumens are illustrated in FIG. 1B, it is contemplated within the scope of this disclosure to include a guide sheath 250 with one, two, three, or more lumens.

In some embodiments, as shown in FIG. 1C, the guide sheath 250 can include a rigid distal end tip 251D2 made of a plastic or metal material and attached to the flexible shaft 251D1 with an adhesive or a thermal fusion process. In some embodiments, the guide sheath 250 also includes stabilizing balloon (not shown) attached at the distal end 251 and enabling the distal end 251 to be fixed in place during a procedure when the balloon is expanded. Additionally, in some embodiments, as shown in FIG. 1C, the guide sheath 250 may be configured to deploy at least one of the imaging probe 50 and the medical tool 200 outward from its distal end 251 in a non-collinear or laterally off-axis manner with respect to the guide sheath 250 (i.e., with respect to a longitudinal axis of the guide sheath 250). In one specific example, the guide sheath 250 may be configured to deploy the imaging probe 50 and/or the medical tool 200 from its distal end at a deployment angle of at least 10 degrees with respect to the longitudinal axis. However, other deployment angles may be contemplated within the scope of this disclosure. The medical apparatus 150 may also include additional structures to facilitate lateral deployment. For example, FIG. 1C shows a bendable flexible tube 201D1 that can slide within an internal lumen of the medical tool 200 (e.g., a dual lumen biopsy needle) and can facilitate off-axis deployment of the imaging probe 50, as discussed in more detail below.

It is to be noted that use of separate and detachable guide sheaths, imaging probes, and/or medical tools is not necessary in some embodiments. Accordingly, in some embodiments, at least one of the imaging probe 50, the guide sheath 250, and the medical tool 200 are attached to or integrated with each other. It will be also clear from description below that embodiments without medical tools are contemplated within the scope of the present disclosure, for example, for purely diagnostic medical procedures. Additionally, embodiments of the guide sheath 250 that are structured to accept or to be inserted into an “off-the-shelf” third party medical tool are within scope of the present disclosure.

Referring again to FIG. 2A, the guide sheath 250 includes the proximal end, or guide sheath handle, 252 that facilitates handling by a human operator in some embodiments. The guide sheath handle 252 can be a plastic tube dimensioned to be conveniently held by the operator and coupled (e.g., bonded with an adhesive) to the guide sheath shaft 251D1. In some embodiments, the guide sheath 250 is equipped with steering means such as, for example, pull wires. As such, the guide sheath handle 252 can include at least one steering actuator 252R, for example in the form of a geared CAM ring 60R shown in FIG. 2C. The CAM ring 60R can be configured to engage a pull wire (not shown) disposed within the guide sheath. In addition or alternatively, the handle 252 can interface with the console 100 and can incorporate or interface with at least one motor (not shown) that engages the steering actuator 252R. In such embodiments, the motor can be powered and controlled with wires and cables 250P2 connected to the console 100. Accordingly, the guide sheath 250 may be actuated manually or in an automated or semi-automated manner.

With respect to the imaging probe 50, Insert A of FIG. 2A shows an overall perspective view, though not to scale, of an embodiment of the imaging probe 50 including a distal end 51, a proximal end 52, and an outer wall extending from the distal end 51 to the proximal end 52. The distal end 51 can be sized to be insertable into and through a lumen of the guide sheath 250 or a working channel of the medical tool 200 (as shown in FIGS. 1A-1C), and the proximal end 52 can be operatively connected to a drive unit 101 of the console 100 (as shown in FIG. 2B). The imaging probe 50, and its relationship with the drive unit 101 and the console 100, may be similar or identical to that described in U.S. Pat. No. 9,364,167, the entire disclosure of which is incorporated herein by reference. For example, during operation, the imaging probe 50 can receive a non-ionizing interrogating energy, or imaging energy, (such as ultrasound energy and/or optical energy) from the drive unit 101 and then project the imaging energy (e.g., through a transparent portion of the outer wall) toward an ambient medium. The imaging probe 50 can then receive, for example, reflected imaging energy in order to acquire and construct image data. Furthermore, as shown in FIGS. 2B and 2C, the proximal end 52 of the imaging probe 50 can include a rotating mechanical coupler 62 and hub or stationary portion 60 operatively coupled to (e.g., connected with) a stationary outer sheath 54 (e.g., the outer wall) of the imaging probe 50. The mechanical coupler 62 can include a probe connector (not shown) configured to couple the non-ionizing imaging energy between the drive unit 101 and the imaging probe 50. In some embodiments, the mechanical coupler 62 can further couple rotational and/or translational motion from motors of the drive unit 101 to a rotating imaging core (not shown) of the imaging probe 50 (e.g., as described in U.S. Pat. No. 9,364,167).

In some embodiments, the imaging probe 50 also includes steering pull wires (not shown) disposed within the outer sheath 54. As further described below, and with reference to U.S. Pat. No. 9,364,167, the pull wires may function to bend or steer the imaging probe 50 (and/or related components such as a flexible medical tool or flexible guide sheath). Such pull wires may be actuated manually and/or automatically or semi-automatically via motors (not shown) in the drive unit 101. For example, as shown in FIG. 2C, the stationary portion 60 can include at least one slideable wire pull element 60B that can be clamped to a pull wire (not shown). The wire pull element 60B can be actuated with the cam ring 60R that, in turn, can be rotated manually by an operator or can be geared to be engaged by a motor inside the drive unit 101. Also, while the pull wire structure is described with respect to the imaging probe 50, it is noted that similar structures can be used to actuate pull wires disposed in the guide sheath 250, as further described below.

In some embodiments, the imaging probe 50 may include various scanning components to facilitate imaging. For example, in some embodiments, as shown in FIG. 6A, the imaging probe 50 may include internal scanning components, such as a rotating imaging element (e.g., within a transparent outer sheath), that is actuated by a flexible rotary shaft or a torque coil to generate spirally scanned patterns for imaging. The rotary shaft or torque coil can be powered by proximally located motors (e.g., at the drive unit 101). In some embodiments, one or more micro-motors, microelectromechanical systems (MEMS), or piezo- or electrostatic scanning actuators can be disposed at the distal end 51 of the imaging probe 50 to cause movement of the distal end 51 to generate scanning patterns for imaging.

Additionally, as shown in FIG. 5, some embodiments may include a distal, light-activated scanning actuator. More specifically, the imaging probe 50 of FIG. 5 can include an optical energy guide 57B, a focusing element 58, an energy directing element 59, a ferrule 68, a mounting element 69, and a photo-actuated element 57B2. As shown in FIG. 5, the focusing element 58 and the ferrule 68 (which holds the optical fiber 57B) are both attached to the auxiliary mounting element 69, such as a metal or glass tube. The optical energy guide 57B can deliver optical energy 55F and may be, but is not limited to, a single-mode optical (SM) fiber, an elliptical core fiber, a polarization preserving (PM) fiber, a multimode-mode (MM) fiber, a double clad fiber (DCF), a micro-structured or photonic crystal fiber (PCF), a multi-core fiber, fiber bundles, a plurality of separate fibers fabricated by any standard fiber-optic processes, or a combination of any of the above optical waveguides or their splicing in one waveguide. The focusing element 58 transmits at least a portion of the optical energy 55F delivered by the guide 57B to a tissue by concentrating the portion to spatial dimensions required for high-resolution imaging (for example, down to less than 200 μm and preferably less than 20 μm).

The photo-actuated element 57B2 can be secured (e.g., with an adhesive) at its proximal end to the ferrule 68 and at its distal end to the optical energy guide 57B, and the optical energy guide 57B can include a region 57B1 that outcouples at least a portion of the optical energy 55S1 toward the photo-actuated element 57B2. The region 57B1 may be, but is not limited to, a tilted Fiber Bragg Grating fabricated in at least one core of the guide 57B or an angled polished tip of a separate fiber of the guide 57B. The optical energy 55S1 redirected by the region 57B1 can illuminate the photo-actuated element 57B2 which, in response, can change shape and cause the distal tip of the optical energy guide 57B to deflect. In some embodiments, the photo-actuated element 57B2 is a photothermal and photostrictive cantilever made from silicon and coated with metal on the side radially opposite the optical energy guide 57B.

Furthermore, in some embodiments, the console 100 can include an optical modulator (not shown) to modulate the resonance of the energy portions 55F, 55S1, for example, so that they are in resonance with a fiber tip of the optical energy guide 57B, thus permitting a linear scanning pattern for imaging. Also, any embodiments described herein of the image probe 50 may further incorporate an ultrasonic transducer or an array of ultrasonic transducers fixedly disposed in the distal end 51 of the imaging probe 50.

With respect to the medical tool 200, referring back to FIG. 2A, the medical tool 200 includes a proximal end, or handle, 202 and a distal end (not shown) sized to be insertable into and through the guide sheath 250. In some embodiments, as noted above, the medical tool 200 can be integrated with or fixed to the guide sheath 250. The medical tool 200 may also be operatively connected to the console 100 via control lines 250P2, for example, for applications that require vacuum, electrical, RF, MW, optical, ultrasound, and/or laser energy deposition, heating or cooling action, motorized or other automated motion of tool components.

Referring now to FIGS. 3A-3D, embodiments of the invention include various methods of placing the medical tool or the probe to a target and/or various methods of using pre-operative and intra-operative image data of different modalities to guide such placement. For example, as shown in FIG. 3A, the medical tool 200 and the imaging probe 50, within the guide sheath 250, can be delivered to a target endoluminally, that is, via steering within a natural or artificially created lumen or cavity 260 (or a lumen network). In another example, as shown in FIG. 3B, the guide sheath 250 is inserted first through a lumen 260 and then the medical tool 200 is advanced transluminally toward a target. In yet another example, FIG. 3C shows a percutaneous placement, where a needle 150C punctures a skin area 300A to place the medical tool 200 and the imaging probe 50 at a target inside an internal organ 300B. In the example of FIG. 3C, the medical system 150 can be further equipped with detectors 150A to track positions of fiducials 150B attached to the body as well as positions and poses of the needle 150C for proper initial alignment of the medical tool 200 relative to the target.

FIG. 3D shows an example using working channels of an endoscope. More specifically, an endoscope 91 with a steerable distal end 92 can be used to deliver the guide sheath 250, the medical tool 200, and the imaging probe 50 through an endoscope working channel 90. As a result, an endoscopic view of the endoscope 91 can be augmented with image data acquired with the imaging probe 50, as further described below. FIG. 3E shows yet another example, where the imaging probe 50 is delivered to a target endoluminally, while medical tools, such as surgical instruments, are delivered subcutaneously (i.e., through the skin area 300A) via surgical ports 90A, 90B. In some embodiments, with reference to the example of FIG. 3E, a separate endoscope can be used to guide the procedure, and the endoscope's visual guidance can be augmented with image data acquired by the imaging probe 50.

To accurately deliver the medical tool 200 or the imaging probe 50 to a target via the above-described placement methods, at least one of the guide sheath 250, the medical tool 200, and the imaging probe 50 is configured to be steerable. In other words, at least one of the guide sheath 250, the medical tool 200, and the imaging probe 50 possesses an ability to change position or pose (e.g., angular orientation) of their distal ends in response to operator input (manual or automatic) at a corresponding proximal end. FIGS. 4A-4C illustrate steering mechanisms for delivering medical tools and/or probes in accordance with the above-described examples of FIGS. 3A-3E. Reference is also made to the description of steering means provided in U.S. Pat. No. 9,364,167.

For example, at least one of the medical tool 200, the guide sheath 250, and the imaging probe 50 may include a bended or bendable distal end. In some embodiments, this bended distal end component may have a different stiffness than a corresponding straight distal end. By way of example, FIG. 4A illustrates a pre-shaped guide sheath 250 and a straight imaging probe 50. The pre-shaped outer guide sheath 250 may be normally bent or curved at its distal end, but flexible enough to be straightened, and the imaging probe 50 may be substantially stiff. As such, the components can be steered by rotating and axially sliding the guide sheath 250 with respect to the imaging probe 50 (e.g., as shown in FIG. 3A). More specifically, the components may be steered in a substantially straight direction when they are arranged so that the imaging probe 50 extends through the distal end of the guide sheath 250. To turn or change direction, the components can be arranged so that the guide sheath distal end extends past the image probe 50, permitting the distal end to bend. The guide sheath 250 (and/or the imaging probe 50) may also be rotated so that the pre-shaped bend of the guide sheath distal end corresponds to the desired turn angle. This method of steering may be used for guide sheath placement within a tubular network, where the guide sheath 250 is configured to be more flexible than the tube walls of the network, thus permitting the guide sheath 250 to comply with or follow bifurcations of the network in response to appropriate proximal end torque provided by an operator.

According to another example, FIG. 4B shows a flexible guide sheath 250P3 steerable by pull wires 73 (e.g., used to place a flexible medical tool to a target, as shown in FIGS. 3A and 3B). The pull wire 73 can extend at least partially through the guide sheath 250 and be connected to a distal end 250P4 of the guide sheath 250P3 (which may be, for example, a metal tip and may include one or more slotted structures to facilitate bending). The pull wire 73 can be further coupled to an actuator at a proximal end of the guide sheath 250 (for example, to one of the actuators described above with respect to FIGS. 2A-2C). The actuator can pull back the pull wire 73, which in turn can bend the metal tip 250P4 of the guide sheath 250. FIG. 4B further illustrates cross-sections A-A, B-B, and C-C taken along a length of the guide sheath 250 and showing relative component dimensions and their relative positions. For example, at cross-section A-A, the pull wire 73 is routed inside the guide sheath 250, while at cross-section B-B, the pull wire 73 is external to the guide sheath 250. Also, the pull wire 73 attaches to the guide sheath 250 at a contact point more proximal than the point at which cross-section C-C is taken.

Referring now to FIG. 4C, a medical apparatus may include a guide sheath 250, an imaging probe 50, and at least one shape changing element 78. The shape changing element 78 can be made of a shape memory alloy (SMA) or a shape memory polymer (SMP) capable of bending or otherwise changing shape and can be positioned relative to the guide sheath 250 so that such bending or shape change can cause the distal end of the guide sheath 250 to bend or twist in at least one direction. In some embodiments, the shape changing element 78 can be actuated with optical energy 55E, for example, delivered via the imaging probe 50 or a light energy depositing medical tool (not shown). In other embodiments, the shape changing element 78 can be actuated via an energy-delivering medical tool such as an RF or MW ablation probe. Furthermore, in some embodiments, as shown in FIG. 4C, the guide sheath 250 can include a transparent tip 250P6 and a metal shaft 250P5 with slots 250P5S that can facilitate bending of the guide sheath 250 during steering. Reference is also made to the light-activated scanning actuator of FIG. 5, described above.

### Position Calculations

According to some embodiments, the console 100 processes image data acquired by the imaging probe 50 to estimate a “global” and real time position of the probe distal end 51. In, particular, as described in the following paragraphs, the console 100 may be configured to calculate a global or real-time position of the distal end of an imaging probe 50 and/or a distal end of the medical tool 200 relative to a target in tissue during side-view imaging of the tissue using one or more of 1D, 2D, and 3D sub-sets of the image data despite the target being located outside of fields of view of the imaging probe 50 and not directly visualized by the imaging probe 50. Herein, three reference frames will be used: (1) fixed space (inertial frame); (2) a moving distal end of a probe; and (3) patient tissue, i.e., an anatomical structure of interest within a patient body. These three reference frames can be represented with orthogonal sets of unit vectors: (1) [\hat{x}ₛ,ŷₛ,\hat{z}ₛ], referring to the fixed space; (2) └\hat{x}ₚ,ŷₚ,\hat{z}ₚ┘, referring to the probe space; and (3) [\hat{x}ₗ,ŷₗ,\hat{z}ₗ], referring to the tissue space. The vector, \hat{z}ₚ is oriented along the longitudinal axis of a probe distal end 51.

The following description will be better understood by referring first to a structure of a 3D image acquired by the above-described imaging probe 50 of FIG. 6A, As mentioned above, the probe distal end 51 can include a rotating imaging element (for side fields of view) within a transparent outer sheath. As the distal end 51 is repositioned, 3D image data can be acquired from a continuous spiral surface 55S6A2 centered on a trajectory of the distal end 51 and formed by rotating a side exiting beam 55S6A1. Furthermore, by slowly repositioning the distal end 51, the spiral surface 55S6A2 can be approximated by a stack of plane 2D frames formed by rotating the beam 55S6A1 (i.e., a freeze and hop approximation). Thus, in some embodiments, a 3D image can be represented by a stack of the 2D frames, or B-scans Bᵢ[n,m] including 1D A-lines Aₙ[m] of image data points or pixels. Such A-lines and B-scans exemplify 1D and 2D sub-sets, respectively, of 3D image data. Additionally, the A-lines, which represent a reconstructed depth profile of reflected imaging energy, are described in more detail in U.S. Pat. No. 9,364,167 and references within.

When the distal end 51 moves with respect to surrounding tissue, each pixel of a corresponding A-line experiences a phase shift relative to an adjacent A-line due to the Doppler effect. These Doppler phase shifts can be measured by the medical apparatus 150, for example by calculating a Kasai autocorrelation. Results of the Kasai autocorrelation calculations can then be used to form “color Doppler” A-lines Aₙ^{CD}[m], and then to form “color Doppler” scans Bᵢ^{CD}[n,m]. These color Doppler data represent, at each image pixel, velocity components v[n,m] the rotating beam 55S6A1, for every A-line. Apart from the Kasai autocorrelation, other signal processing steps may be used to estimate Doppler shifts and form Doppler maps. These Doppler maps can then be used to estimate a position and a pose (i.e., angular orientation) of the probe distal end 51 using, for example, the method steps shown in FIG. 6B.

More specifically, FIG. 6B shows an exemplary recursive calculation sequence, or estimation algorithm, 450 that uses a previous probe state (i.e., a position and a pose of the probe distal end 51 determined from a previous B-scan) to estimate a current probe state using image data from a current B-scan. The estimation algorithm 450 is based on an Extended or Unscented Kalman Filter that uses a probe state model together with a measurement model, where the measurement model relates Doppler shifts in a current B-scans with a current probe state. An exemplary state model for the process 450 is given by:

\vec{X}ᵢ=(\vec{r}ᵢ,\hat{x}_{p,i},ŷ_{p,i},\hat{z}_{p,i})

\vec{r}ᵢ₊₁=\vec{r}ᵢ+\vec{t}ᵢ

\vec{t}ᵢ₊₁=\vec{t}ᵢ

\hat{ξ}ᵢ₊₁= [symbol] _{z}(θ_{z,i}) [symbol] _{y}(θ_{y,i}) [symbol] _{z}(θ_{z,i})\hat{ξ}ᵢ(ξ=x,y,z)

θ_{ξ,i+1}=θ_{ξ,i}(ξ=x,y,z)

Here, i denotes an iteration step, \vec{X}ᵢ is a stable vector with a distal end position \vec{r}ᵢ and a pose └\hat{x}ₚ,ŷₚ,\hat{z}ₚ┘ᵢ, respectively. Further, \vec{t}=[tₓ,t_{y},t_{z}]^{T} is a translation vector of the distal end at each process step. θ_{ξ,i} denotes rotation of the probe reference frame during an i-th iteration around a principal axis of the moving probe reference frame at a previous i−1 iteration, with [symbol] _{ξ}(θ) further denoting a rotation matrix for a rotation with an angle θ around an axis \hat{ξ}ᵢ. All positional and angular accelerations in the Kalman filter include process noise, though the process noise terms are omitted herein for brevity. It is clear from known teachings of Kalman filters and further examples of methods of position and pose estimations of the invention how to modify the state vector and the process noise in the Kalman filter for each embodiment. For example, a different state model for the calculation algorithm of 450 can be used by redefining a state vector [tₓ,t_{y},t_{z},θ_{z},θ_{y},θ_{z}]^{T} in the moving probe reference.

An exemplary measurement model that relates Doppler shifts and the probe state for each iteration of the process 450 is given by:

vᵢ[n,m]=C(\vec{t}ᵢ·\hat{b}ₙ)+wᵢ[n,m]

\vec{t}ᵢ·\hat{b}ₙ=_{z,i} cos Θ+t_{x,i} sin Θ sin nΔφ+t_{y,i} sin Θ cos nΔφ

Here, C is a conversion factor relating Doppler shifts with Doppler velocities vᵢ[n,m], and \hat{b}ₙ a unit vector directed along the exiting beam 55S6A1, which is associated with n-th A-line. This measurement model implies that the first A-line in a frame is always aligned with ŷₚ_{ᵢ} axis, with Δφ being a rotational angle between successive A-lines, and Θ being a look angle, which is defined as an angle between the probe axis and the exiting beam associated with the n-th A-line. Measurement noises may be included in wᵢ[n,m], which also includes Doppler shifts induced by tissue motion within the patient body.

Referring again to FIG. 6B, in a first step 451, the imaging system 150 acquires image data and forms an i-th B-scan (i.e., processes a B₁ frame). In some embodiments, marks that act as indexing features distributed in or on the outer sheath 250 in a pre-determined pattern may be used in this step to correct or compensate for scanning distortion, such as non-uniformity rotational distortion (NURD). For such compensation, the i-th B-scan is remapped, possibly with image data interpolation, to more regularly spaced A-lines using images of the indexing features. In a next step 452, color Doppler A-lines are constructed. More specifically, Doppler processing is used to generate Doppler image data with corresponding Doppler A-lines. Optionally, to improve signal to noise ratios, the Doppler processing includes averaging with a 1D averaging window aligned along A-lines (“in-scan” direction) or across A-lines (“cross-scan” direction) or with a 2D averaging window. During a next step 453, masking filters may be used to remove regions in the i-th Doppler B-scan with low signal and/or high noise, thus removing these regions from the measurement model in subsequent steps. Also, high flow regions, such as those associated with large blood vessels, can be masked out in this step. In some embodiments, such regions can be determined based on comparing Doppler phase shift value or associated noise in the Doppler maps with predetermined rejection thresholds. In other embodiments, rejection thresholds are determined by analyzing image data in the i-th B-scan, with additional image data from previously analyzed B-scans. Alternatively or in addition, knowledge of feature locations (e.g., blood vessels or lumens with no signal) derived from structural B-scan obtained in step 451 can be applied for masking out low signal, high noise, or high flow regions. Finally, remaining Doppler shifts from unmasked regions are included in a Kalman filter processing step 454, which estimates an updated state of the imaging probe 50 (i.e., the position and the pose of the probe distal end 51).

In the process 450, the pose component of the state vector is uneorrelated with the measurement model. In many clinical applications, a distal end pose is not critical and thus pose parameters can be treated as nuisance parameters in the Kalman filter. In other applications, probe positions can be a priori constrained to a trajectory, thus defining the pose to be a tangential to the trajectory. For example, in navigation in a narrow lumen, the probe state model can be modified to constrain probe positions to a lumen centerline, e.g., by using a distance along the centerline as one coordinate in the position state. Then, tangential angles to the centerline define a pose state of the probe distal end. Other modifications may be made to the state model or the measurement model that allow estimating remaining angular orientations of the probe distal end, as further described below. It should be also noted that, although tissue motion is modeled as noise in the process 450, it can be explicitly included in a process and/or measurement model once a model of tissue motion is known.

Limits of approximating B-scans as plane frames, which in turn limit maximal velocities that can be estimated, may be removed if a Kalman filter incorporates geometrical information about the surface formed by a scanning beam. This surface, which is a set of A-lines similar in shape to the scanned spiral 55S6A2 of FIG. 6A, can be parameterized with A-line number n (for example, as a vector function to each pixel \vec{r}[n,m], the function depending also on the probe state parameters that need to be estimated). A sub-set of image data points with known geometrical relationship with respect to a scene, i.e., a local tissue reference frame, can be selected from this parameterized scanning surface. This sub-set can be included in a measurement model at each step of a Kalman filter. For example, as shown in a process 460 in FIG. 6C, a single A-line or its portion can be selected as such a sub-set at a down-sample interval k from the plurality of acquired A-lines at step 461. Here, the down-sample interval k is a parameter that determines a number of skipped A-lines not included in a measurement model. In the process 460, a measurement model is modified such as

vᵢ[n(i),m]=C(\vec{t}ᵢ·\hat{b}ₙ₍ᵢ₎)+wᵢ[n(i),m]

\vec{t}ᵢ·\hat{b}ₙ₍ᵢ₎=t_{z,i} cos Θ+t_{x,i} sin Θ sin n(i)Δφ+t_{y,i} sin Θ cos n(i)Δφ

where n(i)=n(i−1)+k.

Steps 462-464 of process 460 may then performed similar to the above-described steps 452-454 of process 450.

When the medical tool and a target cannot be simultaneously directly visualized in the probe field of view (FOV), a Kalman filter process can be modified to include degrees of freedom of the medical tool in a state model and a measurement model. When the medical tool is fixed relative to the imaging probe, such modification can be a trivial vector offset added to a probe state vector. In embodiments configured to move the medical tool relative to the imaging probe, a position and a pose of the tool can be estimated by adding independent tool state variables to a process model. Examples of such independent variables include, but are not limited to, a variable protrusion of a needle from the guide sheath or an opening angle of forceps jaws. A measurement model in this case relates Doppler shifts from a tool region in an image data set with the tool state components using a known location of a tool portion in the probe FOV. The process model also includes a known spatial relationship between this visualized portion of the medical tool with a portion of the tool that reaches a target. In particular, a process 470 of FIG. 6D includes first and second steps 471, 472 similar to steps 451 and 452 of process 450, respectively. At a third step 473, masking filters may be used to select a tool region. Next, at step 474, Doppler image data with corresponding Doppler A-lines is generated. Then, at step 475, Doppler image data with corresponding Doppler A-lines with respect to the tool region is generated. Finally, step 476 is a Kalman filter processing step to estimate new probe and tool states using predictions from the state model and corrections from the measurement model. This method of analyzing tool region in image data, exemplified with a process 470 of FIG. 6D, is not limited to Doppler shift-based measurement models but can be also applied to other measurement models, as long as such process models incorporate a known relationship between observed tool portion and tool working portion.

Referring now to FIG. 7, a probe state can be determined in a process 480 that analyzes speckle correlations of sub-sets of image data. In the process 480, image B-frames are processed and high-noise and low-speckle regions are removed by masking filters at steps 481 and 482, respectively (similar to steps 451 and 453 of process 450). The image B-frames are then divided in a grid of blocks or sub-frames (step 483). Then, each block in a previous B-frame is correlated with blocks of a current B-frame to find locations of the blocks with peak correlation values. These peak locations, i.e., the translation vectors in the moving probe reference frame for blocks that maximize the correlation, can be used to estimate in-frame or in-plane motion parameters (tₓ,t_{y},θ_{z}). For example, the in-plane parameters can be estimated by the Singular Value Decomposition (SVD) of the covariance matrix formed by the blocks translation vectors in the initial B-frame of i−1 step of the process 480 and the target B-frame of i-th step.

Out-of-plane motion parameters (t_{z},θₓ,θ_{y}) can be estimated using decorrelation curves at step 484. A decorrelation curve is a dependence of image correlation between two blocks on a distance between them, which can be determine by pre-procedural calibration or by use of library of decorrelation curves for specific tissue types. Once correlations between corresponding blocks in Bᵢ and Bᵢ₋₁ frames are known from determining the in-plane parameters, decor elation curves can be used to determine elevation distances z between corresponding blocks, for example, by using look-up tables (e.g., stored in the console 100). Then, the out-of-plane parameters can be estimated by finding a plane that corresponds to determined values of elevation distances, for example, in a least-squares method to solve the equation of a plane determined by the elevation values. An example of such estimation is given below:

A plane equation can be written in a vector form as

z=α₁x+α₂y+α₃

\vec{z}= [symbol] [α₁,α₂,α₃]^{T}.

Here, \vec{z} is a px1 vector of elevational block distances, [symbol] is px3 matrix of axial and lateral position of all p blocks in the first two columns and a column of 1's for the last column, and [α₁,α₂,α₃]^{T} are parameters for a plane that approximates the current Bᵢ scan. A solution in the least-square sense for the plane parameters, for a normal vector to the plane, and for axial displacement, pitch and yaw angles (t_{z},θₓ,θ_{y}) is:

$${[ {α}_{1} , {α}_{2} , {α}_{3} ]}^{T} = {( {\overleftrightarrow{M}}^{T} \overleftrightarrow{M} )}^{- 1} {\overleftrightarrow{M}}^{T} \vec{z}$$

$$\hat{n} = \frac{[ {α}_{1} , {α}_{2} 1 ]}{‖ [ {α}_{1} , {α}_{2} , 1 ] ‖}$$

$${t}_{z} = {α}_{3}$$

$${θ}_{x} = a tan ( \frac{{n}_{x}}{{n}_{z}} )$$

$${θ}_{y} = a sin ( {n}_{y} )$$

Then, these six in-plane and out-of-plane parameters are used in the iterative process 480 to calculate a probe position using Kalman filter methods at step 485. The process 480 can be further modified to include independent tool state (as described in FIG. 6D). Also, probe position and pose values estimated by the process 480 of FIG. 7 can be used to construct 3D image data of tissue with sub-sets of 3D data, such as 2D B-frames. This can be done by remapping each pixel of each B-scan to the tissue reference frame with rotation and translation transformation matrices determined by the process 480. In addition, 3D image data obtained with a non-uniformly scanning beam can be remapped using the process 480. For example, image distortions caused by repositioning the probe distal end at a non-constant velocity can be corrected. Thus, a full 3D image of the surrounding tissue can be constructed while the imaging probe is being placed toward a target.

FIGS. 8A-8D illustrate methods and associated structures that allow for determining a position and a pose of an imaging probe during a procedure using pre-procedural image data from various modalities. In addition or separately, pre-procedural measurements of tissue properties that are invariant with respect to tissue motion can be used, at least in part, to estimate intra-procedural probe state. FIG. 8A shows flow chart 490, which is an iteration step in a process of estimating probe state during probe repositioning. In other words, the process 490 is a measurement step in a Kalman filter process. This measurement step 490 uses a set of B frames acquired intra-operatively by the imaging probe 50 and then compares intra-procedural image data from the acquired set with reference image data of tissue containing a target. The reference image data can be, for example, a 3D image data set of CT slices or MRI slices, pre-acquired and stored in a computer memory (e.g., on the console 100 or on a separate component in communication with the console 100). The reference image data can also be a 3D ultrasound or OCT image, or a set of 2D ultrasound or OCT images with known registration of each 2D frame with the tissue (i.e., with a known position of each 2D frame in the tissue reference frame). The reference image data can further be a library of endoscopic images of any modality with known registration to the tissue reference frame, or 3D image data pre-acquired by the imaging probe 50 (or a different imaging probe) before a procedure or during a previous repositioning step in the procedure. The reference image data can be also a fusion of several modalities, such as a 3D CT data set with co-registered ultrasound data or OCT data.

In the process 490, the reference image data is compared with the image data acquired by the imaging probe 50 to find sub-sets of the reference image data with maximal image similarity. This maximization is used to estimate a probe state. The similarity comparison can be based on, analysis of pixel intensity, such as a comparison of a sum of absolute differences in intensity, normalized correlations in the sub-set of image data, mutual information, and so on. The similarity comparison can be based on analysis of gradients of pixel intensities, or on analysis of features and parameters of the features extracted from the image data. An example of a feature is a contour of a lumen, with a sum of the contour pixel exemplifying the lumen perimeter parameter.

The process 490 of FIG. 8A uses, at each iteration step, a transformation of the reference image data. This transformation is associated with a product of a translation and a rotation of the moving probe reference frame being estimated in the tissue reference frame. Slices extracted from transformed reference image data in a step 495 of FIG. 8A are based on selection of image data points with coordinates that correspond to image data point coordinates of intra-operative sub-volume (of step 491) defined in the probe reference frame. Transformations may further include scaling transformations of the reference image data to account for scaling calibration differences between different modalities and also for dimensional differences in pre-operative and post-operative conditions of tissue. The similarity comparison may also include a step (not shown) of creating a virtual endoscopic image from the intraoperative image data set. This step projects all pixels from the sub-volume of step 491 to a virtual camera placed in a position of the probe distal end, and then compares this virtual view with a corresponding virtual image constructed from the reference image data or with a real endoscopic image from a record of endoscopic images.

FIG. 8AA shows a method 490A of determining a probe state without using image similarities of pre-operative and intraoperative image data. Instead, the method 490A is based on selecting a set of locations within a tissue, preferably on a path to a target, and measuring local tissue properties that identify these locations using pre-procedural data. Measured values of these properties are pre-computed and stored in a record of identifiers, with a set of identifiers labeling each pre-selected location. Then, image data acquired by an imaging probe 50 is analyzed intra-procedurally to determine or calculate image parameters that correspond to the identifiers and, thus, to determine a state of the probe 50. For example, a set of measurements of geometrical properties of lumen branches, such as diameters, perimeters or cross-sectional areas (CSAs), shape properties of a branch lumen, branching angles, lengths between bifurcation points, etc., can be used as a set of identifiers for pre-selected locations within a luminal structure (as described in U.S. Pat. No. 9,364,167). Also, a set of measurements of properties of tissue structure, in particular properties of various anatomical landmarks within the tissue, can be used as a set of identifiers. For example, a value of local epithelium thickness or thickness of other tissue layers of an anatomical cavity can be measured. A CSA of blood vessel, a gland, a patch of fibrotic tissue, or another tissue sub-type feature can be measured to construct a set of identifiers. Because the described identifiers are related to geometrical properties of tissue, they may be referred to as geometrical identifiers. Also, a presence or absence of an anatomical landmark within a pre-determine distance (e.g., associated with a field of view of the probe 50) at each pre-selected location can be used as an anatomical identifier for the location. A non-limiting list of geometrical and anatomical identifiers are provided in a table of FIG. 8B. A defining property of an identifier is a value invariant with respect to tissue translation and rotation. A sub-class of identifiers, exemplified by angles or diameter inheritance factors of branches, i.e., geometrical properties of branches normalized on parent branches, is also invariant to a scaling transformation. Because the identifiers are determined locally, within volumes associated with sub-sets of 3D image data acquired by the probe 50, they are substantially invariant with respect to tissue deformation as well. The use of identifiers is particularly advantageous in, but not limited to, endoluminal and trasluminal placements of medical tools (e.g., as illustrated in FIGS. 3A, 3B and 3E).

Referring back to the method 490A of FIG. 8AA, identifiers can be determined from reference image data, for example from a pre-operative 3D set of CT slices or from a pre-operative image data set acquired by an imaging probe. Identifiers can be pre-measured or pre-calculated using measurement devices such as, for example, balloons, expanding basket catheters, and distance measuring catheters. Identifiers can be also pre-determined from known anatomical considerations for specific tissue structures and tissue organs without a need for any measurements. A measurement of a parameter that corresponds to an identifier in the probe image data is particularly accurate and computationally efficient when the image data contains OCT data or ultrasound data because such data has distance information for each pixel (or image data point). For example, the CSA of a lumen can be measured as a sum of all pixels in an OCT B-scan having an intensity level below a threshold, and the CSA of a blood vessel can be measured as a sum of Doppler pixels in a Doppler OCT B-scan that are above a threshold. A shape parameters of a lumen, such as an asymmetry parameter, can be measured as a ratio of a largest distance between pixels in a B-scan having value below a threshold, to a closest distance between such pixels. A branch length is a pull-back distance between two bifurcation points, where the bifurcation points are defined as B-scans with a lumen asymmetry parameter above a threshold. Once parameters associated with identifiers are measured in the image data acquired by the imaging probe 50, the probe state can be estimated using a look up table that relates one or more sets of identifiers with a set of pre-selected locations. For example, in some embodiments, a score of correlation between the calculated parameters and the pre-computed identifiers can be determined and stored in a table, and the probe state (e.g., the global position of the probe 50) can be calculated based on a comparison between the correlation score with a predetermined acceptance threshold or a record of stored correlation scores. Additionally, the measured parameters enable estimation of probe pose state, as described in more detail below.

In terms of Kalman filters, a pre-selected location can be thought of as a discrete model of a probe state in a Kalman filter, with identifiers and corresponding parameters in the image data related to a measurement model in a discrete space. While the method 490A can be used alone, in some embodiments, it can be also combined with other Kalman filter estimators of probe state described herein. For example, both the Doppler and speckle correlation methods of FIGS. 6B and 7 may have deficiencies in estimating a roll angle θ_{z} of the probe, while the roll angle can be easily determined with the identifier method 490A when identifiers include asymmetry parameters of a lumen or asymmetrically located anatomical landmarks in lumen walls.

This point is further illustrated in FIG. 8C, which shows a schematic of a human lung anatomy as an example of an anatomical luminal structure with substantially asymmetrical luminal properties. Specifically, FIG. 8C shows a bronchial tree 568C1 with corresponding adjacent trees of pulmonary arteries 568C2 and veins 568C3. FIG. 8C also shows three locations of successive B-scans 558C1 (Bᵢ), 558C2 (Bᵢ₋₁), and 558C3 (Bᵢ₋₂), acquired from of a probe being repositioned inside a bronchus. Key features of each B-scan is shown in FIG. 8D, with a bronchus wall contour 568D1 and an adjacent pulmonary vessel 568D2, made visible in the bronchus wall by Doppler signal processing. The image data of FIG. 8D also shows a guide sheath surface with an indexing orientation mark 491A. For simplicity, the image data of FIG. 8D is already transformed to the tissue reference frame. When the probe 50 moves from central airways to more peripheral airways, there is an associated change in geometrical and anatomical identifiers. For example, during such movement, the CSA of an airway decreases and this can be correlated with a position of the probe along a length of the airway. And when the probe 50 approaches a bifurcation point, an asymmetry increases, enabling identification of probe position near a branching point. An angular orientation of a blood vessel with respect to the indexing mark 491A allows estimating a roll angle of the probe with respect to the tissue. Also, an orientation of the asymmetry in the lumen contour can be used to estimate the roll angle. Estimating roll angles enables, in turn, a proper rotation of the guide sheath 250 around its axis (illustrated in successive Bᵢ₋₁ and Bᵢ₋₂ frames) so that the probe 50 enters a correct branch in a bifurcation. Using a combination of the identifiers, such as a combination of one or more of an asymmetry parameter, CSA in several successive B-frames length between bifurcations with a bifurcation detected as a cross-section with an asymmetry value above a pre-determined threshold, blood vessel angular orientation, lumen asymmetry axis, for example in a look-up table, a probe state can be estimated with a high degree of certainty.

It is to be understood that, although the above image data processing steps that estimate a probe position are explained in terms of Kalman filters, other data processing algorithms known in the art of position tracking and able to utilize the above image data sets are contemplated within the scope of the invention.

Referring now to FIGS. 9A-9D, some embodiments may include an imaging probe characterized by a plurality of exiting beams that asymmetrically spread imaging energy in one direction when illuminating a tissue, which can improve probe state estimation in the above-described processing methods. More specifically, the probe designs of FIGS. 9A-9D can be used to determine Doppler shifts for at least two non-collinear beams independently which, in turn, allows for using two components of local tissue velocities in a Kalman filter measurement model. For example, a t_{z} probe state parameter can be estimated independently from tₓ and t_{y} parameters. In addition or alternatively, the designs of FIGS. 9A-9C can have an increased correlation length along the z direction, thus increasing a range of velocities of the probe 50 that can be estimated with the method 480 at a given B-scan acquisition rate.

For example, according to some embodiments, an imaging probe 50 may include a rotating imaging core. As shown in the dual view imaging probe of FIG. 9A, a beamsplitting prism attached to the rotating imaging core can generate non-collinear beams 5591 and 5592. As shown in the probe of FIG. 9B, two separate imaging energy guides or waveguides 5791 and 5792 can be attached to the rotating imaging core and can each generate a non-collinear beam 5591, 5592. Different exitance angles of the beams 5591 and 5592 are produced by beam directing elements with different directing angles for each separate waveguide 5791, 5792. In some embodiments, the imaging probe of FIG. 9B may additionally, or alternatively, include a non-rotating waveguide, separate from a main rotating waveguide. This non-rotating waveguide, together with an associated beam directing element, can be used to estimate a probe state with 1D image data and dedicated (that is, separate from main imaging) Doppler processing or correlation processing. The dedicated Doppler or correlation processing does not require depth information extraction and thus can be implemented with a single wavelength light source.

FIG. 9C illustrates an imaging probe 50, including a waveguide 5791 and a beam directing element 5891, that is configured to spread imaging energy and to generate a number of spots or a continuous line when illuminating a tissue. More specifically, the beam directing element 5891 can include a beamsplitting surface that splits a portion of optical energy 559C1 for side view imaging. The beam directing element 5891 can split another portion of energy 559C2 to be used for measuring Doppler shifts or performing correlation analysis. The portion 559C2 is further split into a number of discrete, non-collinear beams or into a continuum of beams by the element 5891. For this, a surface of the element 5891 that faces a beam directing element 5892 is configured to have a beam shaping property. For example, the surface can have a beam generating transmission grating, can be a cylindrical surface, or can include an attached 1D lens. The beam directing element 5892 sends the portion of energy 559C2 towards a tissue, which can then be processed by a separate data processing channel. Because the portion 559C2 contains non-collinear beams, Doppler shift measurements of this portion can produce a broad spectrum of Doppler frequencies, as shown in FIG. 9D.

More specifically, FIG. 9D illustrates such a broadening for a portion 559C2 having two separate beams. The broadening is characterized by a Doppler spectrum 709D1 with a width 709D2 between a Doppler frequency 709D3 of one beam and a Doppler frequency 709D4 of a second beam, and an overall position of the spectrum 709D1 may be characterized by an average Doppler frequency 709D5. When the probe 50 of FIG. 9C moves with respect to a tissue, movement components along the probe axis produce a broadening of the Doppler spectrum, or a change in the width 709D2, while movement of components orthogonal to the probe axis produce an overall shift of Doppler spectrum or a change in the average frequency 709D5. These two independent measurement values can be used in a state model and in a measurement model of a Kalman filter to improve estimator performance, for example to minimize the influence of tissue motion and deformation.

According to some embodiments, as shown in FIGS. 10A-10C, a probe state can be measured using image data acquired from accelerometers (i.e., acceleration sensors) disposed in the probe distal end 51. For example, as shown in FIG. 10A, an imaging probe 50 can include an imaging core 5310A configured to emit at least two imaging beams 5510A1 and 5510A2. In operation, the imaging beam 5510A1 is transmitted through a transparent outer sheath 5410A to image a surrounding tissue. At the same time, the beam 5510A2 interrogates an accelerometer 5410A2 attached to a cap 5410A1. The accelerometer 5410A2 may be a single-axis accelerometer or a multi-axis accelerometer. FIG. 10B shows an example accelerometer 5410A2, which may be configured to measure acceleration in x-y-z directions with a sensing mass 5419A2c attached to a body 5410A2a via at least one flexible cantilever 5410A2b. When the probe distal end 51 is accelerated in the z direction, the sensing mass 5410A2c is displaced in or out of the plane of FIG. 10B. The sensing mass 5410A2c may also tilt around two orthogonal axes. The beam 5510A detects these three degrees of freedom by imaging features or marks fabricated on a surface of the mass 5410A2c. The system console 100 can then convert these measurements into acceleration values, which are used in a Kalman filter measurement model. In other words, the probe state model can be modified to accommodate accelerometer-based measurements, as is known in the field of tracking and navigation.

In some embodiments, as shown in FIG. 10C, additional accelerometers 5410C1 and 5410C2 may be positioned near or along side walls of the probe 50. The accelerometers 5410C1 and 5410C2 can be used to measure additional parameters of a probe state, such as roll, yaw, and pitch angles. Alternatively, accelerometers disposed in side walls of the probe 50 can be used in place of accelerometers disposed in a forward FOV of the probe 50. In such embodiments, a single beam imaging core can acquire image data through transparent portions of the probe outer sheath between the accelerometers. Alternatively or in addition, the single beam imaging core can be translated along the probe axis to move a scanning imaging beam away from accelerometer to alternate between imaging surrounding tissue and measuring an acceleration of the probe distal end 51.

Turning now to FIGS. 11A-11B, according to some embodiments, position and probe orientation can be determined using image data acquired from a magnetic sensor, such as a magneto-optical or a magneto-strictive element, disposed in the probe distal end 51. For example, FIG. 11A shows an imaging probe 50 with a magneto-optical element 65 disposed at its distal end so that imaging energy passes through the element 65. An external magnetic field can cause a rotation of polarization of light passing through the element 65, and the polarization rotation can be detected by comparing a state of polarization (SOP) of a portion of light reflected from an interface in front of or before the element 65 with an SOP of a light portion reflected from an interface located after the element 65. Here, the terms “before” and “after” refer to light passing time relative to a proximal end of the probe 50. For example, in FIG. 11A, the element 65 is placed between a grin lens 5811A and a prism 5911A. A polarization rotation can be detected by comparing an SOP of an OCT signal associated with a reflection from an interface between the grin lens 5811A and the element 65 and an SOP of an OCT signal associated with a reflection from an interface between the element 65 and the prism 5911A. These SOPs can be determined by methods of polarization sensitive OCT, as described in U.S. Pat. No. 9,364,167 and references within. In operation, at least one external coil or solenoid can be fixed relative to the fixed space reference frame to generate a reference alternating magnetic field. By measuring a time-dependent value of the polarization rotation of the light passing through the element 65 caused by the reference magnetic field, a probe state can be determined. In some embodiments, several magneto-optical elements can be disposed in the probe distal end, with a total number of measurable degrees of freedom equal to a product of a number of external coils and a number of the magneto-optical elements. Alternatively, FIG. 11B shows an imaging probe 50 with a grin lens 5811B a magneto-strictive element 6511B disposed at its distal end. As shown in FIG. 11B, a beamsplitting element 5911B directs one portion of light to image tissue and another portion of light to measure a displacement of the magneto-strictive element 6511B that changes its shape when a magnetic field is applied.

Furthermore, in some embodiments, an electro-magnetic position sensor, for example an Aurora sensor commercially available from Northern Digital Inc., can be disposed in distal ends of the imaging probe 50, the guide sheath 250, and/or the medical instrument(s) 200. The console 100 can be configured to process electrical signals provided by the electro-magnetic position sensor to improve calculation of the probe position.

In some embodiments, a state of the probe distal end can be determined by measuring a present bending state of a bendable structure that, at least partially, encloses the imaging probe 50 or otherwise adapts to a shape that the probe (or a medical tool) takes within a tissue. In such embodiments, the console 100 can be configured to measure the bending state by calculating strain-induced changes in image data acquired by the imaging probe 50 from the bendable structure. A method of estimating a position and an orientation of the probe for navigation in branching luminal structures is described in U.S. Pat. No. 9,364,167. According to embodiments of the present disclosure, however, such methods may be extended to determine a probe state or a tool state in other use cases such as, for example, the probe or the tool placements shown in FIGS. 3B, 3C and 3D.

For example, as shown in FIG. 12A, a distal end of a guide sheath 250 can substantially surround or enclose the imaging probe 50 (which is capable of sliding inside the guide sheath 250). When positioned inside the guide sheath 250, the imaging probe 50 is configured to acquire image data that includes image data of a wire 51412A disposed within a side wall of the guide sheath 250. The wire 51212A has features manufactured along its length, as illustrated in the top view of FIG. 12A. These features may be made, for example by laser cutting, to be detectable in the image data acquired by the probe 50. Thus, when the guide sheath 250 bends, the associated strain in the wire 51412A causes changes in spacing between the features. These changes can then be measured in the image data to determine a bending state of the guide sheath 250 and a position and an angle of the guide sheath distal end. In some embodiments, several wires are disposed within the probe FOV, as shown in a cross-sectional view 250CS, to measure bending angles in multiple orthogonal planes. In addition or alternatively in some embodiments, strain-induced speckle correlation changes are used to determine bending state, as described in U.S. Pat. No. 9,364,167.

Furthermore, in some embodiments, to determine a state of a needle-like medical tool, a liner 5212B shown in FIG. 12B may be placed inside the needle to enclose or surround the imaging probe 50. The liner 5212B can be made from, for example, PTFE and can include a plurality of cut or marked features 5112B. The features 5112B can be imaged by the imaging probe 50 (positioned within the needle) to determine strain-induced changes in spacing between these features and, thus, to determine a bending state of the needle.

The recursive filtering methods described above with respect to FIGS. 6A-12B allow for complementary fusion of different sub-sets or modalities of image data to estimate imaging probe position. These different sub-sets of image data can be also referred to as different channels of image data in a measurement model to emphasize different processing steps and different physical mechanisms underlying acquisition of the image data and subsequent probe state calculations. These different channels are also known as different sensors in the field of Kalman filters. Some image data channels or sensors are referenced to a tissue reference frame (such as, for example, Doppler shifts, in-plane and out-of-plane parameters of correlation analysis, results of similarity analysis, and measurements of identifiers). Other channels or sensors are referenced to a fixed space reference frame (such as, for example, probe acceleration sensors and probe position estimations with respect to an external electro-magnetic field). Also, inaccuracies associated with different image data channels have different spatial scales. Therefore, using a hybrid estimation with sensor fusion or fusion of different channels of image data can improve the accuracy and robustness of probe state determinations. Furthermore, a degree of freedom in a state model can be estimated more accurately with a specific modality in image data, based on a priori assumptions of the state model (e.g., empirical confidence factors) or intra-procedural estimation of measurement model noise in a corresponding channel of image data. Hybrid estimation also allows an automatic co-registration of image data in the fixed space and the tissue reference frames, respectively, for probes that incorporate accelerometers or electro-magnetic sensors.

Referring now to FIG. 13A, according to some embodiments, bias terms may be added to a probe state model in a filter process 491 for a position calculation. These bias terms, known in the art of Kalman filters, can correlate with different channels in a measurement model and can be independently estimated. At each iteration of the filter process 491, sub-sets of image data are acquired and processed as independent channels in a step 491A. A state model with the independently estimated bias terms for additional channels of image data in a measurement model is then constructed in a step 491B. Then, in a step 491C, using extended Kalman filter (EKF), a new probe state is estimated using a prediction from the state model and correction from the measurement model with an adaptive gain matrix based on an estimation of signal to noise ratio in the channels.

The above-described processes of estimating a probe state (described with reference to FIGS. 6A-12B) treated tissue motion as a noise term in a process model. Alternatively or in addition, however, tissue deformation maps can be independently estimated and included in a process 492 of calculating the probe position to a target, as shown in FIG. 13B. In each iteration step of the process 492, a Bᵢ frame is obtained and processed (step 492A) as described before, preferentially using optional indexing features in the sheath to correct for rotational distortion. Then, in step 492B, tissue deformation maps are independently estimated using tissue deformation model and image data. Then, in step 492C, a new probe state with EKF is estimated using prediction from the state model and correction from the measurement model that incorporates the tissue deformation map with the probe state.

The tissue deformation map in the step 492B can be estimated for example using the process of FIG. 6B to obtain Doppler shift differences between pixels in a B-scan. In another example of the process of FIG. 7, translation vectors that maximize correlations of each block of image data correspond to tissue deformation vectors, once an average translation vector is subtracted. Similarly, a non-rigid analysis of similarity of image data is provided in the process of FIG. 8A, and can be used to determine deformation maps that can be included in a process model and a measurement model. Herein, non-rigid analysis may refer to an image data similarity analysis of sub-sets of image data sufficiently small to be considered rigid, with independent transforms of the rigid sub-sets of image data at each iteration step Overall tissue motion relative to the imaging probe can be independently estimated when image data is also acquired from sensors that track position and orientation of a probe relative to the fixed space. A pre-determined model of tissue deformations, in particular tissue deformations caused by an interaction with the medical tool, can be used in a Kalman filter model, for example when a tissue is dragged by a needle traversing the tissue during transluminal or interstitial placements. Overall tissue motion (i.e., relative motion of rigid tissue frame, without deformation, with respect to the space reference frame) can be also included explicitly in the model or can be estimated with external position sensors.

FIGS. 13C and 13D illustrate methods, according to some embodiments, of augmenting images acquired with external imaging devices with image data acquired by an imaging probe 50 using probe state calculations. Subsequent analysis of these augmented images allows further improvements in accuracy and robustness of the probe state estimation. When an imaging modality of an external device is capable Hof direct visualization of an imaging probe with a quantitative determination of the probe distal end position, such an augmentation is a co-registration of the probe image data pixels. Examples of such modalities include, but are not limited to, 3D CT scanners or 3D ultrasound imaging. When an external imaging device is a 2D imaging device, such as an endoscope, an external 2D view is augmented with image data acquired by the imaging probe by tracking a position of a camera of an endoscope (or equivalent imaging components) with a position sensor in the space reference frame, in some embodiments.

With reference to the method 493 of FIG. 13C, first, image data collected by the imaging probe 50 is re-mapped to the space reference frame using a calculated position and an orientation of the imaging probe (step 493A). Then, a position and orientation of an endoscope camera is determined by the position sensor and camera imaging calibration parameters (step 493B). The remapped image data is then projected to a camera view using the camera position and orientation and camera calibration parameters (step 493C).

Alternatively or in addition, an external 2D image can be augmented without a need for independent tracking of an endoscope camera, as shown in the method 494 of FIG. 13D. In these embodiments, at least one feature of the imaging probe is visualized in the endoscope field of view. The feature can be, for example, a light emitting optical element in the imaging probe or a mark on or in the probe's outer sheath. With reference to FIG. 13D, first, image data collected by the imaging probe 50 is re-mapped to the space reference frame (step 494A). The probe feature can then be extracted, for example by segmentation, in the camera field of view to minimize the effects of background image data in subsequent analysis (step 494B). Then, a virtual camera model of the endoscope is constructed that simulates a view of the probe feature from a position of the camera in the space reference frame (step 494C). The virtual camera model can use a position of the probe feature in the space reference frame as determined any of the above-described position determination methods. Next, the simulated camera position is adjusted in the virtual model to maximize similarity between virtual and real endoscopic views (step 494D). Once maximization is achieved, image data acquired by the imaging probe is projected to the camera view using the camera position that maximizes the similarity (step 494E).

To further improve accuracy of the similarity analysis in the method 494, several features substantially spaced apart within the imaging probe can be used in some embodiments. For example, the light emitting element can be repositioned within the guide sheath or within a probe outer sheath to match virtual and real views that correspond to several separate locations of the light emitting element. Also, in some embodiments, the imaging probe can mark a tissue by heating or ablating it with imaging energy in locations known in the space reference frame. These tissue marks can then be visualized by the endoscope and are used in the similarity analysis. In addition, in some embodiments, the medical tool can be configured to deploy a fiducial or to mark a tissue with a dye in a location known in the space reference frame, which can then be visualized by the endoscope and used in the similarity analysis. It should be understood that although an endoscopic camera is described herein with the method 494 of FIG. 13D, other projection-type 2D imaging devices, such as a fluoroscope, X-ray imaging device, or a 2D ultrasound device may be used. It is also clear from the above description that, when an endoscope camera position is determined with a position sensor, the method 494 of FIG. 13D can be used to estimate the probe position in the space reference frame.

Accordingly, in some embodiments, the arrangement of the imaging probe 50 is configured to be steered with a separate endoscope. Yet, in other embodiments the image-guided system is configured to augment an endoscopic image with targets deduced from 3D image data obtained by the imaging probe 50. The arrangement of the imaging probe 50 and a medical tool 200 can be also configured to mark the tissue while the system console 100 can be configured to calculate tissue motion and deformation using these tissue marks observed with an endoscope.

In light of the above, embodiments of the invention provide a console 100 configured to calculate or determine global or real-time probe position based on 1D, 2D, and/or 3D sub-sets of imaging data acquired by side-view imaging. By using only side-view imaging, a size of the imaging probe 50 can be minimized, thus providing more room for medical tools 200 during a medical procedure. Further, by using 1D, 2D, or 3D sub-sets of the 3D imaging data, less computation power and time is required to guide the medical apparatus compared to systems that require using full sets 3D data.

### Extended Fields of View and Real-Time 3D Image Guidance

The following paragraphs describe systems and methods for extended FOV imaging, In particular, FIG. 14A illustrates a cross-section of extended probe FOVs 55S14a1, 55S14a2, 55S14a3 for endoluminal placement of an imaging probe 50. According to a method of some embodiments, a distal end 5114a1 of a probe 50 is repositioned in a lumen 5614a2 (with surrounding tissue 5614a1). In some embodiments, the probe 50 can be repositioned using a steerable endoscope (not shown) that accommodates the probe distal end in a working channel of the endoscope. During repositioning, the probe distal end position is determined or tracked using any of the above-described position calculation methods. Using this position tracking, all image data points in each FOV 55S14a1, 55S14a2, 55S14a3 associated with different locations of the probe distal end have determined coordinates in the tissue reference frame or in the fixed space reference frame. Therefore, by re-mapping or co-registering all the data points to one reference frame, the probe FOV can be extended as a combination or fusion of all FOVs. It should be noted that, while FIG. 14A illustrates an FOV extension for 2D images, 3D image data sets can be fused to generate an extended FOV using the above methods as long as a position of the probe distal at each location is determined.

FIG. 14B illustrates several cross-sectional views of a distal end arrangement of a medical apparatus configured to extend FOV for interstitial placement in tissue (or in lumens). More specifically, the medical apparatus can include a guide sheath 25114b1 and an imaging probe including a puncturing stylet with a distal end 5114B. The stylet distal end 5114B can be directed to separate positions that are spaced apart with respect to a longitudinal axis of the guide sheath 25114b1. In particular, the stylet distal end 5114B can be configured to slide within a hub 20114b2 so that the distal end 5114b can be placed on or off the guide sheath axis. Off-axis placements can be achieved by engaging a bended and slideable (or extendable) element 20114b3, such as a bended NiTi tube, that pushes the stylet distal end 5114B laterally when the element 20114b3 is extended. In some embodiments, the extendable bended element is a curved needle that encloses the distal end 5114B. In other embodiments, other slideable elements may be used that include features to control off-axis placement of the distal end 5114B at predetermined orientations with respect to the guide sheath 25114b1. FIG. 14B further illustrates a medical tool in the form of a biopsy needle 2014b1, however other medical tools can be used in the illustrated arrangement.

FIGS. 15A-15D illustrate configured systems and methods for an extended forward FOV using image data acquired by a probe from a sideway FOV. More specifically, FIG. 15A illustrates a distal end arrangement placed in a luminaltissue structure 5615 with a target T (identified within a pre-procedural reference image data set 5615 of the tissue). The target T may also further include detailed regions of interests ROI. The distal end arrangement can include a needle 20115 inserted in a distal end needle hub 25115b, which is, in turn, inserted into a guide sheath 25115a. Enclosed in the needle 20115 is a distal end 5115 of an imaging probe (shown in Insert A of FIG. 15A). The probe distal end 5115 can be configured to scan surrounding tissue radially or spirally with a side exiting beam 55S15A to form a sideway FOV. The console 100 (operatively connected to the imaging probe) can be configured to calculate a position of the probe distal end 5115 using image data acquired by the probe in real-time from the sideway FOV, in accordance with any of the above-described methods. Using this position, the console 100 re-maps a portion of the pre-operative image data with the target T to the probe reference frame, thus generating a virtual 2D forward view 55F15A with the target T, as shown in Insert B of FIG. 15A, or virtual 3D forward view including the target (not shown).

In some embodiments, such virtual forward views are rendered and updated in real-time based on a position assumed by the imaging probe. For example, such renderings can then be displayed to an operator (e.g., via the console 100) enable improved alignment of the medical tool to the target T. Furthermore, in some embodiments, a virtual model of a distal end of a medical tool can be constructed based on a current position of the medical tool distal end, for example estimated using the process 470 of FIG. 6B. The tool virtual model is then superimposed with the virtual forward image view for real-time virtual visualization of the tool with respect to the target.

FIG. 15B illustrates a miniaturized'flexible endoscope 9015B configured to use the structures and methods described herein for extended forward FOV. As shown in FIG. 15B, the endoscope 9015B can include a guide sheath 25015B with a lumen 25115B sized to accept an imaging probe 5015B. The imaging probe 5015B can be configured to scan surrounding tissue radially within a sideway FOV 55S15B. In addition, the guide sheath 2515B can include a working channel 25115B2 sized to receive or accept other probes or medical tools. The imaging probe 5015B may be in communication with a console 100 configured to calculate a position of a distal end of the probe 5015B using the above-described methods. The console 100 can generate and render a virtual forward view of surrounding tissue from a position assumed by the probe 5015B using pre-procedural reference image data.

Because radially scanning probes can be miniaturized to smaller dimensions than alternative imaging structures, the working channel 25115B2 can be made larger than currently possible with standard endoscopes having the same insertion width as the endoscope 9015B. Alternatively or an addition, larger and therefore improved steering mechanisms can be disposed in the distal end of the endoscope 9015B compared to standard endoscopes (for example, having a larger number of pull wires). In some embodiments, the imaging probe 5015B can also be placed in a medical instrument that is deployed via the working channel 25115B2, at least during some portion of a medical procedure, as illustrated in Insert A of FIG. 15B. As a result, calculation of a position of the medical instrument may be improved by processing image data provided by the inserted imaging probe 5015B for improved virtual visualization of the instrument placement to the target.

FIGS. 15C shows an endoscope 9015C, according to some embodiments, configured for improved transluminal placement of a medical tool. In particular, the endoscope 9015C can be structured to include a guide sheath 25015C that accepts, an imaging probe 5015C within an internal lumen 25115C1. The probe 5015C can be configured for side view imaging, for example to provide cross-sectional views that are portions of cones formed by radially scanning a side exiting beam. The guide sheath 9015C can further incorporate a curved working channel 25115C2 configured to deliver a medical tool at an angle exceeding 10 degrees with respect to the longitudinal axis of the endoscope 9015C. In addition, the working channel 25115C2 can be configured to allow imaging of a portion of the medical tool by the imaging probe 5015C when the tool is inserted in the curved working channel 25115C2. In operation, the endoscope 9015C is first positioned within a lumen to locate a target outside a wall of the lumen. A 3D image of a tissue with the target (i.e., a virtual forward FOV) can then be rendered using any of the above-described methods. Then, a medical tool can be advanced via the working channel 25115C2 while a 3D virtual view is rendered in real time with the tool model superimposed with the virtual forward FOV to enable a virtual visualization of the tool placement to the target.

FIG. 15D shows another endoscope 9015D for improved transluminal placement of medical instruments, including an alternative exit position of the curved working channel. In some embodiments, an imaging probe with an array of transducers instead of mechanical scanning probe may be used with the endoscopes 9015C or 9015D. Furthermore, in some embodiments, an imaging probe can be placed in the medical tool being deployed by the endoscopes 9015C, 9015D and position calculation of the medical tools can be improved by processing image data provided by the inserted imaging probe for improved virtual visualization of the tool placement to the target, as described above with respect to FIG. 15B.

FIG. 16 illustrates several steps of refining pre-operative image data with image data acquired by a probe intra-operatively or using only intra-operative image data. In Step 1, a guide sheath 25116 incorporating an imaging probe 5116 is placed in proximity to a target T within a tissue 5616. Initially, the target T may be determined from a pre-procedural image data or from known anatomical considerations and patient history. Then, in Step 2, the guide sheath 25116 can be withdrawn, leaving the imaging probe 5116 in place to acquire high-resolution image data from the target T while tracking probe distal end positions (in accordance with the methods described above). At this step, detailed regions of interests ROI can be identified within the target T and fused with the pre-operative data using re-mapping to the fixed space reference frame or to the tissue reference frame. Next, at Step 3, a virtual forward view is constructed, as described above, and the imaging probe distal end can be withdrawn to a position where it can image at least a portion of a tissue and a portion of a medical tool 20116, The medical tool 20116 is then aligned to the ROI within the target T using the fused virtual forward view and the instantaneous position of the imaging probe 5116 and the tool 20116, as determined continuously and in real time, by processing image data acquired by the probe 5116 in the sideway field of view.

In light of the above, in some embodiments, sideways FOV image data acquired by an imaging probe intra-operatively is used to generate a virtual forward FOV, thus providing virtual three-dimensional imaging. Furthermore, in some embodiments, both co-registered pre-operative data sets and intra-operative image data acquired by the probe (e.g., 1D, 2D, and/or sub-sets of 3D image data) can be used to generate a virtual forward FOV. The pre-operative image data may be acquired using the imaging probe or other imaging modalities (such as CT slices) and, in some embodiments, may be of lower resolution or include a lower level of details than image data acquired by the imaging probe. The virtual forward FOV renderings may be rendered from the point of view of the imaging probe distal end or the medical tool distal end (e.g., by known or calculated relationships between the imaging probe 50 and the medical tool 200). Furthermore, the imaging probe 50 may be repositioned during imaging, and the console 100 may be configured to fuse together multiple renderings to provide extended or enhanced fields of view. These renderings (single forward FOV or extended FOV) can help track and guide medical tool placement during medical procedures in real time. As discussed above, by using 1D, 2D, or 3D sub-sets of the 3D imaging data, less computation power and time is required to guide the medical tool compared to systems that require using full sets 3D data and, by using side-view imaging, a smaller imaging probe 50 can be used, providing more room for medical tool(s) 200.

### Improved Placement and Alignment of Medical Tools

FIGS. 17A-17F illustrate various medical biopsy device designs configured to collect biopsy samples in a sideway direction, for example in, confined spaces of body lumens and cavities, under real-time guidance of an imaging probe in accordance with the methods described herein.

In particular, FIG. 17A shows a biopsy device with a single lumen flexible shaft 20117A1 that extends from a distal end 20117A to a proximal end 20217A. The shaft 201171 can be sealed at the distal end with a rigid tip 20117A2 (including a cap and a machined cutting window 20117A3), which is bonded to the shaft 20117A1 with an adhesive or via a thermal fusion process. The biopsy device further includes an integrated imaging probe (with a probe distal end 5117A) extendable from the device distal end 20117A. The probe distal end 5117A can be configured to image at least one of a portion of tissue surrounding the device distal end 20117A or an inside portion of the rigid tip 20117A2. In some embodiments, the biopsy device includes a transparent tube 20117A4 positioned inside an internal lumen of the flexible shaft 20117A1 and sealingly secured to a drilled hole in a distal cap of the rigid tip 20117A2. The tube 2017A4 can thus form a lumen, for receiving the probe distal end 5117A, that is isolated from a remaining tissue channel of the internal lumen of the shaft 20117A1. An internal cavity of the rigid tip 20117A2 can be connected to a vacuum port 20217A3 via the tissue channel of the shaft 20117A1.

During a biopsy, the device distal end 20117A can be aligned with respect to a target by pushing, pulling or rotating (i.e., torqueing) the device distal end 20117A, via one or more user inputs at the proximal end 20217A, using image guidance or image feedback provided by the probe distal end 5117A. This image guidance can be a direct visualization of a biopsy target via the cutting window 20117A3. Alternatively or in addition, the image feedback can be a calculated, and a virtual visualization of a biopsy target using image data acquired by the probe to estimate a position of the cutting window 20117A3 with respect to the target can then be rendered. The image data can be obtained by imaging surrounding tissue with the probe distal end 5117A protruding from the device distal end 20117A or with the probe distal end 5117A positioned near a transparent portion of the device distal end 20117A. Once the window 20117A3 is aligned to a target, a vacuum is applied to pull a tissue sample at the target inside the cutting window 20117A3 and into the tissue channel. Then the device distal end 20117A is repositioned (e.g., pulled), to cut the tissue sample away from remaining tissue with the cutting window 20117A3 and the cut tissue sample is collected via the vacuum-activated tissue channel. FIG. 17A also shows the proximal end 20217A including a Y connector 20217A1 that separates the tissue channel and the probe lumen at the proximal end 20217A, a flashing port 20217A2 that can receive and supply liquid to clean the probe distal end 20117A, and a Tuohy Borst adapter 20217A4 that enables proximally activated repositioning of the imaging probe 5117A while maintaining a vacuum in the tissue channel.

FIG. 17B shows another side-collecting biopsy device capable of real-time image guidance. A distal end 20117B of the device includes a flexible multi-lumen extrusion 20117B1 with one internal lumen enclosing a probe distal end 5117B and one or more other internal lumens exposed to cutting windows 20117B3, 20117B4 machined in a rigid tip 20117B2. Each cutting window 20117B3, 20117B4 can be connected to its own source of vacuum via a dedicated tissue channel to anchor a tissue sample when a vacuum is applied, thus allowing independent tissue sample collection. The multi-window arrangement of FIG. 17B minimizes a need to torque the distal end 20117B during alignment to a target, thus improving alignment capabilities of the side-cutting biopsy device.

FIG. 17C shows yet another side-collecting biopsy device with a flexible shaft 20117C1, a proximal end 20217C, and a distal end 20117C. The shaft 20117C1 is bonded proximally to a Y connector 2021701 and distally to a rigid tip 20117C2, which has a stationary window or opening 20117C3. The distal end 20117C includes a moveable internal cutting element or cutting stylet 20117C3, which extends through an internal lumen of the flexible shaft 20117C1 to the proximal end 20217C and can be repositioned by a handle 20217C4 at the proximal end 20217C. The cutting stylet 20117C3 can be manufactured from a capped metal tube by machining a cutaway feature that forms a cutting edge and the stationary window 20117C3 to facilitate biopsy sample removal. Accordingly, when a tissue is prolapsed inside the stationary window 20117C3, a user can pull the stylet 20117C3 to cut a tissue sample. An imaging probe 5117C can be coupled to the cutting stylet 20117C3, for example with an adhesive, and is configured to image the window 20117C4. In some embodiments, the imaging probe 5117C is further configured to image a surrounding tissue via an imaging window 20117C5 machined in the stylet 20117C3. FIG. 17C also shows the proximal end 20217C including a Tuohy Borst adapter 20217C3 configured to accept the imaging probe distal end and a vacuum port 20217C2.

FIG. 17D shows yet another side-collecting biopsy device with a moveable internal cutter. The device includes distal end 20117Da with a stationary window 20117D2 machined in a rigid tip 20117D1 that is sealed with a cap 20117D3. The rigid tip 20117D1 can be bonded to a flexible shaft (not shown) of the device. The device can further include an internal cutter 20117D4 made of a metal tube with a machined moveable cutting window 20117D5. Alternative shapes of stationary and moveable windows 20117D2b and 20117D5b are also shown in distal end arrangement 20117Db. An imaging probe (not shown) can be slideably disposed inside the cutter 20117D4, for example, to image the windows 20117D2, 20117D5 (or 20117D2b, 20117D5b).

FIG. 17E shows yet another side-collecting biopsy device with a rotating external cutter 20117E2. The rotating cutter 20117E2 can be proximally actuated via a flexible drive shaft 25117E1 and is configured to rotate inside a stationary (non-rotating) tip 25117E4 but extend from a distal end thereof. An imaging probe 5117E can be disposed inside the rotating cutter 20117E2 and image data can be acquired through cutting openings of the cutter 20117E2 or slots machined in the tip 25117E4. In some embodiments, the imaging probe includes a non-rotating side-imaging core attached to the rotating cutter 20117E2. Image data is thus acquired through the openings or slots by rotating the cutter 20117E2 (i.e., from its proximal end). The arrangement of FIG. 17E may also be configured to articulate the distal tip 25117E4 using one of the above-described steering mechanisms. For example, the biopsy device may be configured with the ability to press the cutter toward a tissue using a pull wire 25117E3 that articulates a portion of the stationary tip 25117E4. Alternatively, other steering mechanisms can be used to bend the articulating segment of the stationary tip 25117E4.

Similar to the device of FIG. 17E, FIG. 17F illustrates a side-collecting biopsy device with a rotating internal cutter 20117F2 enclosed in non-rotating rigid tip and configured to have a fixed and articulating segments. The device can include a flexible drive shaft 25117F1, the internal cutter 25117F2, one or more pull wires 25117F3, and an imaging probe 5117F. The device of FIG. 17F has the internal moveable cutter 20117F2 protected by the non-rotatable rigid tip, making this device safer to use than the device of FIG. 17E.

Referring now to FIGS. 18A-18F, FIGS. 18A-18F illustrate various medical biopsy device designs configured to collect biopsy samples in a forward direction under real-time guidance of an imaging probe in accordance with the methods described herein.

In particular, FIG. 18A shows a forward-collecting biopsy device distal end arrangement with an imaging probe distal end 5118A, a notched tissue puncturing stylet 20118A2, and a cutting cannula 20118A1. These elements are coaxially slideable relative to each other and disposed distally in a flexible outer sheath 25118A1 having a bonded distal outer tip 25118A2. The distal tip 25118A2 may be steerable (e.g., by including at least one fixed portion and at least one articulating portion actuated with a pull wire or other steering element). In some embodiments, the distal rigid tip 25118A2 only includes fixed portion, while a flexible shaft has a pre-formed bended shape to facilitate biopsy device placement. Also, in some embodiments, the flexible outer sheath 25118A1 may be used without a distal outer tip.

FIG. 18B shows a forward-collecting core or aspiration biopsy needle device including a multi-lumen needle 20118B1 with one lumen configured to accept tissue samples and another lumen configured to accommodate a probe distal end 5118B. To facilitate imaging of surrounding tissue, e.g., to confirm placement within a target, the needle device may also include an imaging slot 20118B2 in some embodiments.

FIG. 18C shows a forward-collecting biopsy device including a coring needle 20118C1 and an imaging probe 5118C configured to be positioned laterally offset from a longitudinal axis of the needle 2011801. Such off-angle placement can be achieved using a bended element 20118C2, such as a beaded nitinol tube, that guides the probe 5118C laterally when extended from the needle 20118C1. The device further includes a flexible outer sheath 25118C1 with a rigid distal tip 25118C2 from which the needle 20118C1 extends. A keying element 25118C3 may be attached to the needle 20118C1 and configured to engage corresponding internal keying features (not shown) in the tip 25118C2 to lock an angular orientation of a lateral off-angle placement of the probe 5118C1. To change an angular orientation of the probe 5118C1, the needle 20118C1 can be pulled away from the tip 25118C2 so that the keying element 35118C3 disengages from corresponding tip keying features, then the needle 20118C1 can be rotated and pushed back toward the tip 25118C2 to reengage the keying features in a different angular orientation. Additionally, while FIG. 18C shows a multi-lumen needle (e.g., with one lumen dedicated to tissue collection), a single lumen needle can be used. In particular, the imaging probe 5118C can be positioned inside the needle lumen until a confirmation of a correct placement is obtained, then the imaging probe 5118C may be removed. In some embodiments, the bended element 2018C2 is a needle configured to aspire and collect tissue samples or deliver therapeutic or marking agents when the probe 5118C is removed.

FIG. 18D shows a forward-collecting biopsy device configured to laterally place an integrated imaging probe during a biopsy procedure. A distal end of the biopsy device can include a flexible outer sheath 25118D1, a flexible needle 20118D1 with a side window and an internal slideable stylet hub 20118D2 that stabilizes a slideable inte rnal flexible tube 20118D3, and an imaging probe or imaging stylet 5118D. The biopsy device can further incude an element 25118D2 slideable over the needle 20118D1. The element 25118D includes a protruding bended feature configured to slide inside a notch machined in the needle 20118D1 and to push against the internal tube 20118D3. Thus, when the element 25118D is pulled back, the bended feature is not engaging the notch in the needle 20118D1 and the imaging stylet 5118D extends straight, co-axially with the needle 20118D1. However, when the element 25118D is pushed forward, the internal tube 20118D3 can be bent by the element 25118D. As a result, the bent internal tube 20118D3 can guide the imaging stylet 5118D to extend laterally or off-angle via a side exit window when the imaging stylet 5118D is pushed from its proximal end. In some embodiments, the side exit window in the needle 20118D1 can be covered with a bendable lip to facilitate tissue extraction when the imaging stylet is removed. In some embodiments, the slideable internal flexible tube 20118D3 is a needle configured to aspire and collect tissue samples or deliver therapeutic or marking agents when the imaging stylet 5118D is removed. Additionally, in some embodiments the slideable internal flexible tube 20118D3 can be deployed to puncture a wall of a lumen.

FIG. 18E shows a distal end of medical device configured to build a tunnel or artificial lumen in a tissue towards a target, under the guidance of an imaging probe of some embodiments. The medical device includes a flexible shaft 20118E2 configured to be pushable and torqueable from its proximal end. The flexible shaft 20118E2 is bonded distally to a rigid tip 20118E3, which can include machined cutting features configured to cut tissue and remove tissue debris via internal lumens of the flexible shaft 20118E2 when the flexible shaft 20118E2 is pushed or torqued. The rigid tip 20118E3 is also configured to accept coaxially an imaging probe or stylet 5118E1 that can slide inside a guiding tube 20118E4. The guiding tube 20118E2 has a bended shape and can slide and rotate inside the tip 20118E3. This combination of imaging stylet 5118E1 and guiding tube 20118E4 permits a user to search for a target inside the tissue using directly acquired image data, extended FOVs, and/or additional fused imaging modalities, as described above. In some embodiments, the imaging stylet 5118E1 can include sharpened cap 5118E2 that facilities tissue penetration. Accordingly, when a target is confirmed, the combination can act as a guide wire to guide the tip 20118E3 (e.g., a tunnel digging tip) toward the target. Once the target is reached, a guide sheath 25118E1 can be slid over the flexible shaft 20118E2 until the guide sheath 25118E1 reaches the target.

FIG. 18F shows a distal end of a medical device including a pre-bended or active needle. The device can include a flexible outer sheath 25118F1 with a distally attached rigid tip or rigid hub 25118F2. The outer sheath 25118F and the hub 25118F2 can be configured to accommodate a needle 20118F1 so that the needle 20118F can be repositioned inside the outer sheath. More specifically, the needle 20118F1 can include a keying element 20118F2 configured to engage corresponding keying features in the hub 25118F2 to stabilize angular orientation of the needle 20118F1 during placement. The needle 20118F1 is further configured to accept an imaging probe 5118F1. In some embodiments, the needle 20118F1 has a pre-bended shape and is configured to have a lower stiffness than the, hub 25118F2. Alternatively or in addition, the needle 20118F2 is configured to be actively bended by means of an internal pull wire or an internally disposed shape memory element configured to be activated with energy (e.g., heat) delivered by the probe 5118F1.

Referring now to FIGS. 19A-19C, FIGS. 19A-19C illustrate energy depositing treatment devices with improved accuracy of placement to a treatment target under real-time guidance of an imaging probe in accordance with the methods described herein.

In particular, FIG. 19A shows a cross-section of a MW ablation device. This device, at its distal end 20119A, can include a flexible multi-lumen extrusion 25119A1 with one or more transparent portions to allow transmission of imaging energy from a probe distal end 5119A. The extrusion 25119A1 includes a lumen that accommodates the probe distal end 5119A and a lumen that accommodates a microwave antenna 20119A1 in communication with a proximally located source of microwave energy (e.g., via a microwave cable disposed in the extrusion lumen). The extrusion can also include sealed internal lumens dedicated for cooling liquid. The distal end 20119A further includes a steering element 25119A2, such as a pull wire or thermally activated shape memory element (as described above). In some embodiments, the extrusion lumen accommodating an imaging probe is open so that the probe distal end 5119A can slide beyond the extrusion to be used as a guide wire for placement of the distal end 20119A.

FIG. 19B shows an MW ablation device with improved interstitial placement capabilities. In particular, the device can be configured to place a distal end arrangement 20119B (structured similar to the arrangement of 20119A of FIG. 19A and including a microwave antenna 20119B1 and a steering element 25119B2) with a needle interstitially in a tissue target. In some embodiments, the device is capable of being steered to off-angle positions (illustrated with dotted lines in FIG. 19B) and image data fusion (as described above) can be used to increase FOV to improve margin identifications of the treatment target. In some embodiments, a separate MW ablation probe can be delivered via a dedicated lumen in a needle once a target is identified with the off-angle placed imaging probe, and the needle can then be re-aligned toward the target.

FIG. 19C shows a cross-section of a device, such as a laser ablation device, a photo-dynamic therapy device, or a light treatment device. The device, at its distal end, can include a multi-lumen extrusion 25119C1 with a steering element 25119C2 and an imaging probe 5119C. The steering element 25119C2 can be a pull wire or a shape memory element activated by light, as described above. The imaging probe 5119C can be configured to deliver optical imaging energy as well as treatment doses of optical energy. In some embodiments, the imaging probe 5119C can include light diffusing elements.

### First Example Medical Procedure

The following paragraphs and FIGS. 20A-21C describe a first example medical procedure, in accordance with embodiments of the invention, which is a non-surgical bronchoscopic biopsy of lung nodules found by chest CT. In particular, FIGS. 20A-20D illustrate procedure steps, while FIGS. 21A-21C illustrate relationships between image data used to determine positions of biopsy tools and corresponding virtual or augmented views that can facilitate image guidance. This bronchoscopic lung tissue biopsy is an endobronchial or a transbronchial example of the endoluminal or transluminal placement of a medical tool illustrated in FIGS. 3A-3B, respectively.

FIG. 20A illustrates a flowchart of a bronchoscopic biopsy procedure 700 of lung nodules using a guide sheath, a probe, and biopsy tools of certain embodiments. FIG. 20A also shows how different image data sets are used to guide the biopsy procedure 700. First, when a patient is referred for a biopsy of a suspicious, indeterminate lung nodule found by a chest CT, a practitioner (such as an interventional pulmonologist) determines, in a planning step 710, a pathway to the nodule (also called a lesion) in the patient airway tree. The planning phase 710 can use 3D pre-procedural image data 780, for example constructed from slices of the chest CT that detected the nodule. The pre-procedural image data 780 may also include additional CT data acquired immediately prior to the biopsy procedure, or data acquired with other imaging modalities such as OCT, ultrasound, or MRI. An output of the planning step 710 is a rendering of a 3D airway tree model from the pre-procedural data set 780, identifying a target representing the nodule and a pathway through airways to the target, as illustrated in in FIG. 21A.

During the planning step 710, the practitioner also determines an appropriate set or combination of a guide sheath, an imaging probe, and biopsy tools to be used during the biopsy procedure 700. This selection of biopsy devices is based on dimensions of a bronchus (or bronchi) leading to the lesion and on a bronchus-to-lesion spatial relation. For example, in a favorable case of a mucosal lesion surrounding a bronchus that can accommodate a biopsy tool, a side cutting biopsy tool, such as the one shown in FIG. 17A, might be sufficient. In other cases of a lesion tangential to or located deeper with respect to an airway, a forward tissue collecting biopsy tool, such as the one shown in FIG. 14B, can be used in addition to or instead of the side cutting tool. Generally, to improve biopsy yield, the practitioner may use the side cutting tool followed by the forward collecting tool. Also, in addition to the biopsy tools, the practitioner may select a pre-bended separate guide sheath similar to the one shown in FIG. 4A and a separate side view imaging probe configured to perform circumferential or pull-back spiral scanning during imaging. Alternatively, to increase procedure speed, the practitioner may choose to use a single device for the biopsy procedure, such as the transbronchial device of FIG. 18D that allows straight or off-angle deployment of tissue collecting needles without a need to exchange tools, as further described below with reference to step 760.

Referring again to FIG. 20A, an intervention portion of the biopsy procedure 700 begins with a standard guided bronchoscopy at step 720, where the practitioner brings a bronchoscope as close as possible to the lesion via the pathway determined in step 710, as shown in FIG. 21A. Preferably, the bronchoscopy 720 uses Virtual Bronchoscopy Navigation (VBN) or Electro-magnetic Navigation (EMN). Alternatively, clinicians may rely on their own knowledge of airways and use unguided bronchoscopy in step 720. Once the bronchoscope cannot advance any further, the imaging probe in the guide sheath is brought into more peripheral airways, via a working channel of the bronchoscope, in an extended VBN step 730. In step 730, the imaging probe and the guide sheath are advanced towards the target with imaging guidance from image data collected by the probe, for example, using the steering method shown in FIG. 4A.

Referring now to FIG. 20B, the extended navigation step 730 includes steps 731-733 of “making turns” in the branching airway tree and an optional step 734 of “looking around” for accurate localization within the airway tree, While any method of position calculation described herein can be used in step 731, a preferred method of determining a position of the probe is based on the method 490A of FIG. 8AA. More specifically, with reference to FIGS. 20B and 21B, at step 731, intra-procedural data 795 (such as 1D, 2D, and/or sub-sets of 3D image data) is acquired in real-time by the imaging probe and used to calculate probe position. Pathway identifiers 790 are pre-computed from the pre-procedural data 780 and then updated based on the intra-procedural data 795. The pathway identifiers 790 may be geometrical identifiers chosen from the group consisting of an airway branch (i.e., a bronchus) diameter, a bronchus length, a bronchus lumen asymmetry parameter, and a size and orientation of a pulmonary artery adjacent to a bronchus. For example, during step 731, the geometrical identifiers can be measured from a 2D cross-sectional image data acquired in real-time by the imaging probe (in the sideway field of view of the probe), as illustrated in FIG. 21B.

Optionally, at step 732, a virtual forward view of a bifurcation located in front of the imaging probe is rendered using the pre-procedural data 780, preferably refined and augmented with the intra-procedural data 795. This virtual forward view, illustrated in FIG. 21B, can help the practitioner to steer the probe, in step 733, toward a target child branch of the bifurcation. Optionally, in step 734, a pull-back imaging is performed to “look around,” that is, to acquire a 3D image of airway segments that include several bifurcations and sub-surface puhnonary blood vessels. Correlating geometrical identifiers measured with a 3D pull-back data set with the pre-computed and stored library of identifiers 790 enables a high-confidence localization of the probe in the 3D airway tree model, as illustrated in FIG. 21B. This pull-back image data, and particularly images of the pulmonary blood vessels, is also used to update the pathway identifiers for subsequent re-passages of the bifurcation, for example in a follow-up endobronchial procedure. At step 735, probe position can be double checked to ensure the probe is properly positioned in the desired pathway. If not, the imaging probe can be repositioned back to the previous bifurcation, the target child branch can be reassigned, and the method can revert back to step 733. If the imaging probe is correctly positioned, the method can continue to a next bifurcation on the planned route.

Referring back to FIG. 20A, once navigated to a vicinity of the lesion, the probe acquires a high-resolution 3D image data set (step 740) of one or several airway segments located within the lesion or in proximity to the lesion. The practitioner can review the image data 795 to identify biopsy targets in an image analysis step 750. The image data 795 can also be stored to be passed to a pathologist to assist histological analysis of biopsy tissue samples. Then, the probe is exchanged with the biopsy tools to perform an endobronchial biopsy 760, followed by a transbronchial biopsy 770, as further described below.

Referring now to FIG. 20C, biopsy samples can be first obtained with the side cutting tool in the endobronchial procedure 760. The side cutting tool is navigated, in a navigation step 761 similar or identical to the extended VBN step 730 of FIG. 20A, to the airways where the biopsy targets were identified in step 740 of FIG. 20A. During the navigation step 761, an integrated imaging probe of the side cutting tool is extended beyond the tool metal tip for real-time circumferential imaging. Once the side cutting tool is placed in the target airway, the side cutting tool biopsy window is aligned to the biopsy target at step 762. This alignment 762 is accomplished by rotating and translating the side cutting tool from its proximal end. During the alignment step 762, the internal probe can be repositioned to visualize tissue within the distal tip and provide real-time feedback. To further facilitate alignment to biopsy targets, the real-time image data can be augmented with the practitioner marks identifying the biopsy targets. Once the side cutting tool is aligned, a vacuum is applied to the tissue channel to anchor the side cutting tool to the biopsy target at step 763. Then, tissue prolapsed in the cutting window is cut by pulling the side-cutting tool from its proximal end while confirming proper tissue collection with integrated probe imaging (step 764). Finally, the side-cutting biopsy tool is removed from the patient lungs and remaining tissue samples are extracted from the tool tissue channels at step 765. In some embodiments, use of a separate imaging probe in step 740 can be omitted and the biopsy procedure 700 can use a combination side cutting tool imaging device in the steps 730, 740 and 750, thus minimizing the number of tool exchanges.

Referring now to FIG. 20D, the transbronchial procedure 770 with the forward collecting biopsy tool (e.g., a flexible needle biopsy tool) can be performed. The transbronchial procedure 770 may be necessary when the lesion is located deeper in parenchyma and, as a result, cannot be accessed with a side cutting tool. For example, the flexible needle tool can be used when biopsy targets cannot be identified with an endobronchially placed imaging probe because cancerous tissue sites of a lesion are located beyond the depth of view of the imaging probe. The transbronchial procedure 770 may also be used in addition to the endobronchial biopsy 760 to improve accuracy of a biopsy by sampling more regions within a lesion.

In the transbronchial needle biopsy 770, once navigated to a carina in a vicinity of the lesion (step 771), the needle tool is aimed towards an initial biopsy target in step 772. The aiming step 772 can use the intra-procedural data 795, which at this point is fused with the pre-procedural data 780 (not shown), to construct a virtual forward view with the initial biopsy target, as illustrated in FIG. 21C. The fusion of the pre-procedural CT data allows aiming to a target located deeper within or beyond airway walls, beyond the imaging depth of the probes. Also, in some embodiments, fusion of image data acquired with probes of different modalities, such a radial endobronchial ultrasound probe, facilitates image guidance. At the same time, the use of intra-procedural data 795, namely a higher resolution data acquired by a probe, allows estimation and tracking of the needle position within the lungs with higher accuracy. The needle position tracking is preferably implemented by the method 490 of FIG. 8A using image data acquired in real-time in side view by the imaging probe (e.g., an imaging stylet) integrated with the needle biopsy tools, and the position tracking is used to update the virtual forward view of the target.

The aiming 772 is actuated by steering the distal end of the device with the guide sheath (e.g., by repositioning the pre-bended distal end of the guide sheath from its proximal end). Once aimed, the needle is extracted from the distal end hub to puncture the airway wall at step 773, by pushing the flexible needle from its proximal end (for example, as shown in the wall puncture panel section of FIG. 18D). Referring back to FIG. 20D, the needle biopsy procedure 770 uses an interstitial image acquisition 774 of the lesion. In step 774, the imaging stylet advances beyond the coring needle to acquire interstitial image data, preferably using the above-described methods of extending FOVs (for example, with reference to FIG. 14B). This interstitial or parenchymal imaging is used to update the intra-procedural image data set 795. Next, the updated image data 795 is reviewed and analyzed by the practitioner to identify updated biopsy targets (step 775). At step 776, the imaging stylet is retracted and the needle is re-aligned towards the biopsy targets. For example, the stylet is removed and the needle can be advanced towards the biopsy targets while applying a vacuum to obtain core tissue samples. In a final step 777, the needle tool is removed and biopsy tissue samples are extracted from the needle biopsy tool for subsequent histopathology. The transbronchial procedure 770 can be also used to collect tissue samples from lymph nodes to stage lung cancer. Alternatively or an addition, transbronchial biopsy tools that allow wall penetration with an off-angle needle placement (as described above) can be used in the transbronchial procedure 770.

### Second Example Medical Procedure

The following paragraphs and FIGS. 22A-23 describe a second example medical procedure, in accordance with embodiments of the invention, which is a surgical resection of a small lung nodule with an intraoperative localization of the nodule assisted or guided with an imaging probe. More specifically, the surgical resection is a video-assisted thoracic surgery (VATS) or robotically-assisted thoracic surgery (RATS), which are known to have deficiencies in localization of sub-centimeter or non-solid lung nodules, especially located deeper than 10 mm from a pleural surface. Therefore, it is highly desirable to assist a surgeon in localization of small nodules during VATS/RATS, thus decreasing operation duration and minimizing complication risks.

FIG. 22A illustrates a method 800 of improved surgical resection of a small lung nodule, with endobronchial and transbronchial intraoperative nodule localization. Preferably, the nodule has been biopsied prior to the surgical procedure 800 with the bronchoscopic biopsy procedure 700 of FIG. 20A. For example, bronchoscopic biopsies and VATS resections using devices of some embodiments can be included in a lung nodule management algorithm. Referring back to FIG. 22A, pre-procedural image data 780, intra-procedural data 795, and pathway identifiers 790 are all available prior to the surgical procedure 800 and are used in a planning step 810. The planning step 810 involves, for example, planning locations of surgical ports and selection of surgical tools. During the planning step 810, the surgeon also determines if any advanced nodule localization techniques or sentinel lymph node mapping are required. For a difficult-to-localize nodule, the surgeon may choose between a transbronchial nodule marking 820 or an endobronchial nodule localization 830, or the surgeon may plan to use both. The surgeon can also plan to use a fiducial disposing device in addition to or instead of marking the nodule with a dye.

After planning, surgical intervention starts by performing, in the operative settings, a bronchoscopic transbronchial placement of a flexible needle device (similar to the flexible needle biopsy tool described above) to the lesion using the extended VBN method described above. Once placed in the lesion, the flexible needle device injects a nodule marking dye. For example, a fluorescent marking dye, such as ICG dilution mixed with albumin, is injected at step 820 and the dye is configured to diffuse to sentinel lymph nodules within a time period, such as 10-20 minutes. The marking dye is used to localize the nodule or sentinel lymph nodes during VATS/RATS in a thoracoscope FOV. Also, the marking dye is used to localize the sentinel lymph node in a resected specimen to ensure proper surgical margins. Alternatively or in addition, a fiducial is deployed in the lesion via an internal lumen of the flexible needle. Such a fiducial can be an expandable coil or an expandable pre-loaded metal tube that self-anchors in an airway or in parenchyma. Optionally, an internal imaging probe of the flexible needle tool is used to confirm a proper placement of the fiducial in the lesion.

Next, the flexible needle device is removed and endobronchial intraoperative nodule localization begins at step 830, as shown in FIG. 22B. More specifically, for intraoperative nodule localization 830, an imaging probe in a guide sheath is navigated to a nodule position under real-time image guidance (step 831). The nodule position is in an airway near the lesion and is pre-determined during the surgery planning. If the nodule position is located far from the pleural surface, the probe in the guide sheath or just the probe may be advanced further to a pre-determined pleural position (step 832) to facilitate direct intraoperative visualization of the probe in a thoracoscope field of view (step 833). During the nodule localization, the imaging probe emits light visible to the thoracoscope. The emitted light may also include components that excite fluorescence of the marking dye. The probe can be repositioned between the pleural and the nodule positions during nodule localization to help the surgeon establish spatial relationships for surgical tool placements. Alternatively or in addition, the thoracoscope FOV can be augmented with a virtual position of the probe in the nodule or pleural position using the above-described methods to facilitate the localization (step 834).

Referring back to FIG. 22A, a guided VATS/RATS step 840 involves imaging confirmation of proper placement of the imaging probe in the nodule and pleural locations once the lungs are deflated. The VATS/RATS procedure step 840 further involves localization of the probe light-emitting distal end directly in the thoracoscope FOV or in the augmented FOV, as shown in FIG. 23. Once the nodule is visually localized with light emitted by the probe, the surgeon can grab a deflated lung tissue portion, including the imaging probe in the guide sheath, with a surgical tool. Then, the probe is removed and the lung tissue with the guide sheath is resected. The resected tissue can be analyzed to ensure that the nodule is completely removed and that the resected tissue contains all sentinel lymph nodes identified by the diffused marking dye. Alternatively, with a fiducial deployed at step 820, the light-emitting imaging probe can guide the surgeon to the fiducial location. Once the fiducial is located, the probe and the guide sheath are withdrawn and the surgeon grabs the fiducial to ensure that the nodule is fully resected.

### Third Example Medical Procedure

The following paragraphs and FIG. 24 describe a third example medical procedure, in accordance with embodiments of the invention, which is an image-guided bronchoscopic microwave (MW) ablation procedure 900 of a cancerous lung, nodule.

The procedure 900 is first planned in a planning step 910 that uses pre-procedural data 780, preferably co-registered with intra-procedural data 795. The intra-procedural data 795 is acquired during a previously performed endobronchial or transbronchial biopsy. Next, an endobronchial MW device, such as the device shown in FIG. 19A, is navigated to the nodule using a guided bronchoscopy 920 followed by an extended VBN 930. Then, the endobronchial MW device is placed as close to a center of the lesion as airways allow at step 940. At step 950, a practitioner determines if the nodule margins are safely within margins of sufficient MW energy deposition using a calculated position of the endobronchial MW device relative to the nodule. In steps 940 and 950, the nodule margins can be determined from both direct imaging and from virtual imaging derived from the co-registered data 780, 795. The range of sufficient deposition of MW energy may be pre-determined based on known tissue properties and MW exposure time. If the lesion is within the margins, the nodule is ablated. If no airway can be found that places the endobronchial MW device in a position to reliably ablate the lesions while minimizing damage to a healthy tissue, the endobronchial MW device may be replaced with a transbronchial MW device, such as the device shown in FIG. 19B, at step 960. At step 960, the transbronchial MW device is placed to the lesion center and the tumor is ablated. Alternatively or in addition, an artificial airway toward the tumor can be constructed, for example using the device shown in FIG. 18E, to properly position the MW ablation device. Also, in some embodiments, the device shown in FIG. 15B can be used to deploy an MW ablation catheter to a target using any of the above-described image-guided methods.

### Other Considerations

At least some elements of a device of the invention can be controlled, in operation with a processor governed by instructions stored in a memory such as to enable desired operation of these elements and/or system or effectuate the flow of the process of the invention. This memory may be random access memory (RAM), read-only memory (ROM), flash memory, or any other memory or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g., read-only memory devices within a computer, such as ROM, or devices readable by a computer I/O attachment, such as CD-ROM or DVD disks), information alterably stored on writable storage media (e.g., floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the disclosed inventive concepts. In this disclosure, embodiments have been described in a way that enables a clear and concise specification to be written, but it'is intended and will be appreciated that elements/components of related embodiments may be variously combined or separated without parting from the scope of the invention, In particular, it will be appreciated that any and all features described herein may be applicable to all aspects of the invention.

Furthermore, disclosed aspects, or portions of these aspects, may be combined in ways not listed above. Accordingly, the invention should not be viewed as being limited to the disclosed embodiment(s). In particular, references throughout this disclosure have been made to “one embodiment,” “an embodiment,” “a related embodiment,” or similar language. Such references mean that a particular feature, structure, or characteristic described in connection with the referred-to “embodiment” is included in at least one embodiment of the present invention. Thus, appearances of the phrases “in one embodiment,” “in an embodiment,” and similar language throughout this specification may, but do not necessarily, all refer to the same implementation of the inventive concept. Furthermore, the phrases “in another embodiment” and “in some other embodiments” as used herein do not necessarily refer to a different embodiment, although it may.

It is to be understood that no portion of disclosure, taken on its own and in possible connection with a figure, is intended to provide a complete description of all features of the invention, It is also to be understood that no single drawing used in describing embodiments of the invention is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

The invention as recited in claims appended to this disclosure is intended to be assessed in light of the disclosure as a whole, including features disclosed in prior art to which reference is made.

1. An image-guided system comprising:
an imaging probe configured to image a tissue, the imaging probe including:
an elongated flexible body having a proximal end, an opposite distal end, and an outer wall extending from the proximal end to the distal end, at least a portion of the outer wall being at least partially transparent to imaging energy used for side-view imaging by the imaging probe,
an energy guide extended inside the flexible body and configured to deliver the imaging energy between the proximal end and the distal end,
at least one energy directing element configured to transmit the imaging energy delivered by the energy guide to the tissue;
a guide sheath including an elongated flexible body with a proximal end, an opposite distal end, and a channel extending from the proximal end to the distal end, the channel configured to accept the imaging probe and a medical tool; and
an imaging console including a data-processing unit in operable communication with the imaging probe and configured to:
process the imaging energy acquired by the image probe during side-view imaging to generate image data, and
calculate a global position of one of the distal end of the imaging probe and a distal end of the medical tool relative to a target in the tissue during imaging of the tissue using any one of 1D, 2D, and 3D sub-sets of the image data despite the target being located outside of fields of view of the imaging probe and not directly visualized by the imaging probe.

2. An image-guided system according to claim 1, wherein the imaging console is configured to calculate the global position using one of Doppler signal processing and correlation analysis of image speckles using the 1D, 2D, and 3D sub-sets of the image data.

3. An image-guided system according to claim 1, wherein the imaging console is configured to calculate the global position based on a comparison of the 1D, 2D, and 3D sub-sets of the image data with pre-acquired reference image data.

4. An image-guided system according to claim 1, wherein the imaging console is further configured to:
(i) pre-compute sets of tissue identifiers at each location of a designated set of locations within the tissue, the tissue identifiers and the set of locations being based on pre-acquired reference image data of the tissue;
(ii) calculate a parameter in the image data acquired with the imaging probe, the parameter corresponding to at least one of the pre-computed tissue identifiers;
(iii) determine and store a score of correlation between the calculated parameters and the pre-computed identifiers; and
(iv) calculate the global position based on a comparison between the score of correlation with at least one of a predetermined acceptance threshold and a record of stored correlation scores.

5. An image-guided system according to claim 1, further comprising a bendable element adaptable to a shape of at least one of the medical instrument and the imaging probe, wherein the imaging console is configured to calculate the global position based on determining a present shape of the bendable element.

6. An image-guided system according to claim 1, wherein the distal end of the imaging probe includes at least one of an acceleration sensor and a magnetic sensor.

7. An image-guided system according to claim 1, wherein at least one of the imaging probe, the medical instrument, and the guide sheath includes an electro-magnetic position sensor; and wherein the imaging console is configured to processes electrical signals from the electro-magnetic position sensor to obtain position data.

8. An image-guided system according to claim 1, wherein the guide sheath includes a steering mechanism configured to deflect the distal end of the guide sheath, the steering mechanism including one of: (a) a wire disposed within the body of the guide sheath; (b) a bended shape of the distal end of the guide sheath; and (c) a shape memory element disposed in the guide sheath and in thermal contact with a portion of the guide sheath, the shape memory element configured to change its shape when said portion of the guide sheath is heated.

9. An image-guided system according to claim 1, wherein the imaging console is configured to calculate the global position, in part, by mapping tissue deformation.

10. An image-guided system comprising:
an imaging probe configured to image a tissue, the imaging probe including:
an elongated flexible body having a proximal end, an opposite distal end, and an outer wall extending from the proximal end to the distal end, at least a portion of the outer wall being at least partially transparent to imaging energy used for side-view imaging by the imaging probe;
an energy guide extended inside the flexible body and configured to deliver the imaging energy between the proximal end and the distal end, and
at least one energy directing element configured to transmit the imaging energy delivered by the energy guide to the tissue;
an imaging console in operable communication with the imaging probe and configured to:
process imaging energy acquired by the imaging probe to generate and store first image data,
calculate a real-time position of the distal end of the imaging probe during imaging of the tissue using any of 1D, 2D, and 3D sub-sets of the first image data, and
render a virtual image of tissue around the distal end of the imaging probe by remapping pre-acquired reference image data of the tissue according to the real-time position of the distal end of the imaging probe.

11. An image-guided system according to claim 10, wherein the imaging console is configured to calculate the real-time position based on one of Doppler signal processing and correlation analysis of image speckles using the 1D, 2D, and 3D sub-sets of the first image data.

12. An image-guided system according to claim 10, wherein the imaging console is configured to calculate the real-time position based on a comparison of the 1D, 2D, and 3D sub-sets of the first image data with at least one of the reference image data and second set of pre-acquired reference image data.

13. A navigational system according to claim 10, wherein the imaging console is further configured to:
(i) pre-compute sets of tissue identifiers at each location of a designated set of locations within the tissue, the identifiers and the set of locations being based on the reference image data of the tissue;
(ii) calculate a parameter from the first image data, the parameter corresponding to at least one of the pre-computed identifiers of the tissue;
(iii) determine and store, in a stored record of correlation scores, a score of correlation between the calculated parameters and the pre-computed identifiers; and
(iv) calculate the real-time position based on comparison between the score of correlation with at least one of a predetermined acceptance threshold and a record of stored correlation scores.

14. An image-guided system according to claim 10, further comprising a bendable element adaptable to a shape of at least one of the medical instrument and the imaging probe, wherein the imaging console is configured to calculate the real-time position based on determining a present shape of the bendable element.

15. An image-guided system according to claim 10, wherein the distal end of the imaging probe includes at least one of an acceleration sensor and a magnetic sensor.

16. An image-guided system according to claim 10, wherein at least one of the imaging probe, the medical instrument, and the guide sheath includes an electro-magnetic position sensor; and wherein the imaging console is configured to processes electrical signals from the electro-magnetic position sensor to obtain position data.

17. An image-guided system according to claim 10 and further comprising a guide sheath including an elongated flexible body having a proximal end, an opposite distal end, a first channel extending from the proximal end to the distal end and configured to accept the imaging probe, and a second channel extending from the proximal end to the distal end and configured to accept a medical tool.

18. An image-guided system according to claim 17, wherein the imaging probe is further configured to obtain second image data from a portion of the medical tool inserted in the second channel; and the console is further configured to:
process the second image data to calculate a real-time position of the distal end of the medical tool, and
render a virtual forward view around the distal end of the probe with a superimposed virtual image of the medical tool.

19. An image-guided system according to claim 17, wherein the guide sheath includes a steering mechanism configured to deflect the distal end of the guide sheath, the steering mechanism including one of (a) a wire disposed within the body of the guide sheath; (b) a bended shape of the distal end of the guide sheath; and (c) a shape memory element disposed in the guide sheath and in thermal contact with a portion of the guide sheath, the shape memory element configured to change its shape when said portion of the guide sheath is heated.

20. An image-guided system according to claim 17, wherein the guide sheath is further configured to deflect one of the imaging probe and the medical tool at at least a 10 degree angle with respect to a longitudinal axis of the guide sheath.

21-27. (canceled)
